(19) United States
(12) Patent Application Publication
(10) Pub. No.: US 2018/0289666 A1
(43) Pub. Date: Oct. 11, 2018

(54) FLAVONOLIGNANS FOR TREATMENT OF AUTOIMMUNE INFLAMMATORY DISEASES

(71) Applicant: Macau University of Science and Technology, Taipa (MO)

(72) Inventors: Ying Xie, Taipa (MO); Liang Liu, Taipa (MO); Hua Zhou, Taipa (MO); Zhong-Wen Yuan, Taipa (MO); Yan-Fang Zheng, Taipa (MO); Sen-Ling Feng, Taipa (MO); Xiao-Hui Zeng, Taipa (MO); Hui Wang, Taipa (MO)

(21) Appl. No.: 15/481,720

(22) Filed: Apr. 7, 2017

(51) Int. Cl. A61K 31/366

(52) U.S. Cl. CPC A61K 31/366

(57) ABSTRACT

A method for reducing abnormalities in lipid metabolism and for reducing inflammation in a subject suffering from an autoimmune inflammatory disease accompanied by abnormalities in lipid metabolism includes administering an effective amount of a flavonolignan to the subject. The subject may additionally suffer from a liver disease, obesity, hypertension, diabetes mellitus or a metabolic syndrome. Further provided is a method for reducing the risk of a cardiovascular disease in a subject suffering from an autoimmune inflammatory disease accompanied by abnormalities in lipid metabolism and a method for reducing hepatic abnormalities and reducing inflammation in a subject suffering from an autoimmune inflammatory disease accompanied by hepatic abnormalities. Still further provided is a method for reducing liver damages associated with the treatment of rheumatoid arthritis with a disease-modifying antirheumatic drug or a non-steroidal anti-inflammatory drug.

### SEQUENCE LISTING

The Sequence Listing file entitled “sequencelisting” having a size of 10,895 bytes and a creation date of 7 Apr. 2017 that was filed with the patent application is incorporated herein by reference in its entirety.

### TECHNICAL FIELD

The present invention relates to a method for reducing abnormalities in lipid metabolism and for reducing inflammation in a subject such as a mammal, in particular a human, suffering from an autoimmune inflammatory disease, in particular an autoimmune arthritis especially preferably but not exclusively rheumatoid arthritis, accompanied by abnormalities in lipid metabolism, in particular a decreased level of HDL cholesterol with a normal or mildly increased level of total cholesterol, a normal or mildly increased level of LDL cholesterol and a normal or mildly increased level of triglycerides and optionally further accompanied by hepatic abnormalities. Said method comprises administering an effective amount of a flavonolignan to said subject. The subject may additionally suffer from a liver disease, obesity, hypertension, diabetes mellitus or a metabolic syndrome. Further provided is a method for reducing the risk of a cardiovascular disease in a subject suffering from an autoimmune inflammatory disease accompanied by abnormalities in lipid metabolism and a method for reducing hepatic abnormalities and reducing inflammation in a subject suffering from an autoimmune inflammatory disease accompanied by hepatic abnormalities. Still further provided is a method for reducing liver damages associated with the treatment of autoimmune inflammatory diseases such as rheumatoid arthritis with a disease-modifying antirheumatic drug or a non-steroidal anti-inflammatory drug by administering a flavonolignan.

### BACKGROUND OF THE INVENTION

The world's incidence and prevalence of autoimmune inflammatory diseases such as rheumatoid arthritis (RA) is dramatically rising. RA is characterized by a chronic synovial inflammation and cartilage and bone destruction. It is a relapsing autoimmune disorder which often affects multiple systems and organs including vascular tissues, liver, and brain. The prevalence of RA is around 1 to 2% of the world population. Nearly two-third of the patients with RA are overweight or obese and have metabolic abnormalities accompanied by insulin resistance. Moreover, there are strong evidences supporting an association between RA disease activity and metabolic syndrome (MetS) in patients, and MetS might determine the inflammation milieu leading to the occurrence of more severe RA.

Particularly, changes in lipid profiles and lipoprotein pattern described as “lipid paradox” in the blood of RA patients have been commonly observed and recognized as the major risk factor of increased cardiovascular disease (CVD) in RA. Studies in patients with preclinical RA and early RA demonstrated a lipid profile that is typical of metabolic syndrome: normal or mildly elevated total cholesterol, LDL cholesterol and triglycerides, associated with decreased HDL cholesterol levels. By contrast, the development of highly active RA has been observed for being associated with decreased total cholesterol and LDL cholesterol levels.

In recent studies, improved lipid levels were noticed with RA treatment including conventional DMARDs and biological agents. Drugs acting on lipid/glucose metabolisms appear to confer an improvement on inflammation features in RA patients in recent completed clinical trials. Therefore modulating the lipid metabolism as well as lipid related pathway could be a new potential treatment option to improve both the inflammatory status and the CVD outcome in RA patients. Abnormalities in lipid and lipoprotein metabolism accompanied by chronic inflammation are considered to be the central pathway for the development of nonalcoholic fatty liver disease (NAFLD) which may progress to non-alcoholic steatohepatitis (NASH). Insulin resistance, adipocytokines, proinflammatory cytokines, oxidative stress and lipid peroxidation are believed to be the major causes of progression to NASH which are very similar as the pathogenesis of RA. Therefore, it is not surprising that these two latter diseases may co-exist within same individual. Evidence of prevalence rate of NAFLD in the RA patients was reported with a rate at 23% in the U.S. which is higher than the rate of 15% in non-RA patients with similar comorbidities by ultrasound. Diagnosing the NAFLD or NASH in the RA population with liver biopsy is a reliable approach but not done routinely as it is an invasive and costly procedure. One study in unselected series of RA patients showed that none specific reactive hepatitis was recognized in 43% and fatty changes in 22% of RA population with liver biopsy specimens. This prevalence went to even higher (79%) in RA patients who had clinical and/or biochemical evidence of hepatic dysfunction. Although raised alkaline phosphate (ALP) was observed in about 50% of RA patients, a rise in transaminases in serum was very rare, which make fatty liver disease difficult for physicians to manage because they can be present for years before becoming clinically apparent.

Unfortunately, a wide spectrum of hepatotoxicity has been described with antirheumatic and anti-arthritis drugs such as disease-modifying antirheumatic drugs (DMARDs) and nonsteroidal anti-inflammatory drugs (NSAIDs), which has been an important safety concern for a long term treatment in patients with RA. Several first line therapeutic agents approved for RA treatment such as methotrexate (MTX), and/or leflunomide are associated with increased elevation of liver enzymes, steatohepatitis and fibrosis of the liver as well as other undesirable side effects. For example, there is also strong evidence for methotrexate (MTX), the first-line disease-modifying anti-rheumatic drug, associated with nonalcoholic fatty liver disease with transaminitis in a cohort of RA patients. As the liver is one of the largest lymphoid organs involved in the immune response and a central organ in lipogenesis, gluconeogenesis and cholesterol metabolism, improving the liver function with modulation of metabolic process such as lipid metabolism might be helpful for increasing the host defense efficiently and improving the clinical outcome of RA and/or NAFLD.

Data on this topic are limited and the role of statins in RA remains unclear, there are increasing data that lipid-lowering therapy with statins suppresses RA activity and inflammatory factors and is associated with a lower risk of mortality among patients with RA (Steiner, G. and Urowitz, M. B., Semin Arthritis Rheum, 2009, 38(5): p. 372-81, McCarey, D. W., et al., Lancet, 2004, 363(9426): p. 2015-21, Mowla, K., et al., J Clin Diagn Res, 2016, 10(5): p. OC32-6) as well as NAFLD (Fon Tacer, K. and Rozman, D., J Lipids, 2011, p. 783976). Despite established roles of statins in treatment of RA, significant gaps remain in view of the mechanism related to the metabolic changes in lipid profiles of RA and the role of lipid modulation in the treatment of RA.

Silybin with its stereoisomers Silybin A and Silybin B is the main active compound in Silymarin which is a unique flavonoid complex derived from the milk thistle plant Silybum marianum and one of the most famous liver protective natural products (Feher, J. and Lengyel, G., Curr Pharm Biotechnol, 2012, 13(1): p. 210-7). The positively efficacy of Silybin on lipid metabolism and live protection has been demonstrated (Gobalakrishnan, S., et al., J Clin Diagn Res, 2016, 10(4): p. FF01-5, Suh, H. J., et al., Chem Biol Interact, 2015, 227: p. 53-62, Serviddio, G., et al., J Pharmacol Exp Ther, 2010, 332(3): p. 922-32, Loguercio, C. and Festi, D., World J Gastroenterol, 2011, 17(18): p. 2288-301). The effects of Silybin on lipid metabolism and hepatic abnormalities in RA patients have, however, not been evaluated so far.

Accordingly, there remains a strong need for further compounds effective for treating autoimmune inflammatory diseases, in particular for treatment of RA, which can reduce abnormalities in lipid metabolism and cardiovascular risk of subjects suffering from such a disease without significant side effects such as to the liver as major drawback of usual therapeutic compounds used in the treatment of autoimmune inflammatory diseases.

### SUMMARY OF THE INVENTION

The first aspect of the present invention relates to a method for reducing abnormalities in lipid metabolism and for reducing inflammation in a subject such as a mammal, in particular a human. Said method comprises administering an effective amount of a flavonolignan to said subject. Said subject suffers from an autoimmune inflammatory disease, in particular an autoimmune arthritis such as rheumatoid arthritis, accompanied by abnormalities in lipid metabolism, in particular a decreased level of high-density lipoprotein (HDL) cholesterol with normal or mildly increased levels of total cholesterol, of low-density lipoprotein (LDL) cholesterol and of triglycerides.

The subject may further have hepatic abnormalities and/or suffer from a liver disease or metabolic abnormalities accompanied by insulin resistance such as a metabolic syndrome, obesity, diabetes mellitus or hypertension. The method can further include reducing the risk of a cardiovascular disease such as ischemic heart disease, heart failure, myocardial infarction, stroke or sudden cardiac death and/or reducing hepatic abnormalities of the subject.

The flavonolignan is in particular a lipid-modulating and hepatoprotective flavonolignan, i.e. the flavonolignan is a lipid-modulator with hepatoprotective function and, thus, improves liver function with modulation of lipid metabolism.

The flavonolignan may include one or more compounds having Formulas (Ia), (Ib), (IIa), (IIb), (IIIa) and/or (IVa) or glycosides, salts or solvates thereof:

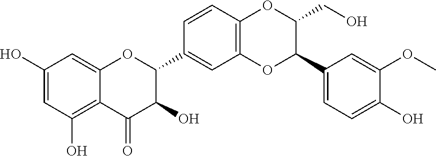 [SMILES: COc1cc([C@H]2Oc3cc([C@H]4Oc5cc(O)cc(O)c5C(=O)[C@@H]4O)ccc3O[C@@H]2CO)ccc1O]

Silybin A which is known for having Formula (Ia);

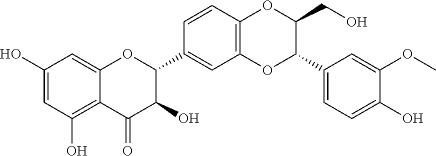 [SMILES: COc1cc([C@@H]2Oc3cc([C@H]4Oc5cc(O)cc(O)c5C(=O)[C@@H]4O)ccc3O[C@H]2CO)ccc1O]

Silybin B which is known for having Formula (Ib);

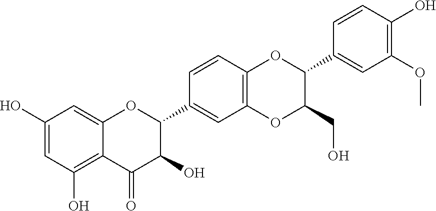 [SMILES: COc1cc([C@H]2Oc3ccc([C@H]4Oc5cc(O)cc(O)c5C(=O)[C@@H]4O)cc3O[C@@H]2CO)ccc1O]

Isosilybin A which is known for having Formula (IIa);

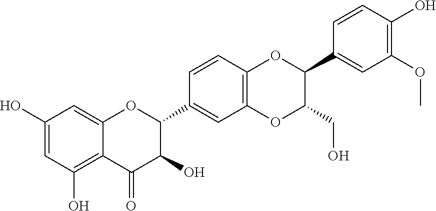 [SMILES: COc1cc([C@@H]2Oc3ccc([C@H]4Oc5cc(O)cc(O)c5C(=O)[C@@H]4O)cc3O[C@H]2CO)ccc1O]

Isosilybin B which is known for having Formula (IIb);

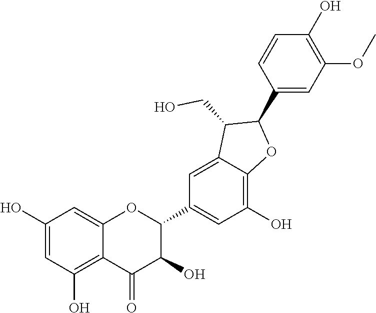 [SMILES: COc1cc([C@H]2Oc3c(O)cc([C@H]4Oc5cc(O)cc(O)c5C(=O)[C@@H]4O)cc3[C@@H]2CO)ccc1O]

Silychristin which is known for having Formula (IIIa);

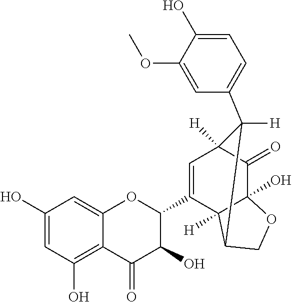 [SMILES: COc1cc(C2C3CO[C@]4(O)C(=O)[C@H]2C=C([C@H]2Oc5cc(O)cc(O)c5C(=O)[C@@H]2O)[C@H]34)ccc1O]

Silydianin which is known for having Formula (IV).

The flavonolignan in particular is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both.

The present invention in a second aspect provides a method for reducing the risk of a cardiovascular disease in a subject comprising administering an effective amount of a flavonolignan to said subject such as a human, wherein the subject suffers from an autoimmune inflammatory disease, in particular an autoimmune arthritis such as RA, accompanied by abnormalities in lipid metabolism.

The subject in particular has an elevated Erythrocyte Sedimentation Rate (ESR), in particular a significantly increased ESR. The abnormalities in lipid metabolism in particular include a decreased level of HDL cholesterol with normal or mildly increased levels of total cholesterol, of low-density lipoprotein (LDL) cholesterol and of triglycerides. Alternatively, abnormalities in lipid metabolism can also include a decreased level of HDL cholesterol, of total cholesterol and of LDL cholesterol with increased levels of triglycerides.

The method in particular comprises reducing the risk of ischemic heart disease including angina, of heart failure, myocardial infarction, stroke and/or sudden cardiac death due to the reduction in abnormalities in lipid metabolism and reduction in inflammation by administering the effective amount of the flavonolignan. The flavonolignan in particular is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both.

Still further provided by the present invention is a method for reducing hepatic abnormalities and reducing inflammation in a subject such as a human. Said method comprises administering an effective amount of a combination of a flavonolignan and a disease-modifying antirheumatic drug, in particular methotrexate, to said subject. Said subject suffers from an autoimmune inflammatory disease accompanied by hepatic abnormalities and in particular additionally by lipid metabolism abnormalities.

The flavonolignan is in particular a lipid-modulating and hepatoprotective flavonolignan and in particular is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both. The hepatic abnormality in particular includes increased levels of Alkaline Phosphatase (ALP) and/or Aspartate Aminotransferase (AST).

Still further provided by the present invention is a method for reducing liver damages associated with the treatment of an autoimmune inflammatory disease such as rheumatoid arthritis with a disease-modifying antirheumatic drug or a non-steroidal anti-inflammatory drug. Said method comprises administering an effective amount of a flavonolignan to said subject before, after or simultaneously with the disease-modifying antirheumatic drug such as methotrexate or non-steroidal anti-inflammatory drug.

The methods of the present invention represent a highly promising treatment option for patients with autoimmune inflammatory diseases, in particular RA. The inventor could in particular show that Silybin (mixture of Silybin A and Silybin B) has significant pharmacological anti-inflammatory, lipid modulating and liver protection effects which make the treatment in particular advantageous for RA patients with fatty liver disease. Oral administration of Silybin in particular proved to significantly reduce swelling, bone erosions, inflammation and liver injury without appearing toxicity in AIA arthritis and a MCD-diet induced NASH model, indicating that it provides a highly promising and advantageous treatment for autoimmune inflammatory diseases with protection of liver function.

Those skilled in the art will appreciate that the invention described herein is susceptible to variations and modifications other than those specifically described. The invention includes all such variations and modifications. The invention also includes all steps and features referred to or indicated in the specification, individually or collectively, and any and all combinations of the steps or features.

Other features and aspects of the invention will become apparent by consideration of the following detailed description and accompanying drawings.

### BRIEF DESCRIPTION OF THE DRAWINGS

FIGS. 1A through 1I refer to AIA rats treated with Silybin. FIG. 1A shows the body weight of AIA rats treated with Silybin (50:50 (w/w) mixture of Silybin A and Silybin B). FIG. 1B shows the arthritic scores of AIA rats treated with Silybin. FIG. 1C shows the hind paw volumes of AIA rats treated with Silybin. FIG. 1D shows the ESR of AIA rats treated with Silybin. Rats were treated daily either with an oral administration of 100 or 200 mg/kg Silybin (referenced as “SB100” and “SB200”, respectively), or vehicle (“Model”), or with an oral dose of 7.6 mg/kg methotrexate (MTX) beginning on day 0 after immunization until day 42 compared to a control group (normal rats, “Control” or “Ctr”). FIG. 1E shows the effect of Silybin on protein expression of cytokine IL-1β in the plasma of AIA rats after 42 days treatment. FIG. 1F shows the effect of Silybin on protein expression of TNF-α in the plasma of AIA rats after 42 days treatment. FIG. 1G shows the effect of Silybin on protein expression of MMP-9 in the plasma of AIA rats after 42 days treatment. FIG. 1H shows the effect of Silybin on protein expression of TIMP-1 in the plasma of AIA rats after 42 days treatment. FIG. 1I shows the effect of Silybin on protein expression of PGE2 in the plasma of AIA rats after 42 days treatment. Data are expressed as means±S.E.M (n=10-11). ^{#,}*p<0.05, ^{##,}**p<0.01, ^{###,}***p<0.001 versus normal rats or versus the vehicle-treated rats.

FIGS. 2A through 2D show the effects of Silybin on the clinical signs, radiological changes and histologic lesions of the hind paws of AIA rats. FIG. 2A shows representative photo images of the hind paws from normal, arthritis AIA rats (“Model”) and arthritis AIA rats treated with MTX or Silybin at 100 mg/kg (“SB100”) or 200 mg/kg (“SB200”) taken on day 42 after induced AIA. FIG. 2B shows radiographs of the hind paws from normal, arthritis AIA rats and arthritis AIA rats treated with MTX or Silybin at 100 mg/kg or 200 mg/kg taken on day 42 after induced AIA. FIG. 2C shows histopathological images of the hind paws from normal control, arthritis AIA rats and arthritis AIA rats treated with MTX or Silybin at 200 mg/kg taken on day 42 after induced AIA. Intense edema and erythema with severe soft tissue swelling and bone erosion were observed in the hind paws of the AIA rats compared with normal rats which were markedly reduced with the treatment of MTX and Silybin. Representative hematoxylin and eosin-stained sections (magnification, ×40) revealing histopathological changes in the tibiotarsal joints of the AIA model rats. FIG. 2D is a diagram showing the respective histological score in normal and arthritis AIA rats (“Model”) and the treatment groups with MTX or Silybin at 200 mg/kg (“SB200”).

FIGS. 3A through 3M show the effects of Silybin and MTX on the liver function and lipid profile in AIA rats after 42 days treatment. FIG. 3A refers to the level of ALT in arthritis AIA rats in the absence (“Model”, vehicle-treated rats) and presence of MTX and Silybin (“SB100” and “SB200”). FIG. 3B refers to the level of AST in arthritis AIA rats in the absence and presence of MTX and Silybin. FIG. 3C refers to the level of ALP in arthritis AIA rats in the absence and presence of MTX and Silybin. FIG. 3D refers to the level of GGT in arthritis AIA rats in the absence and presence of MTX and Silybin. FIG. 3E refers to the level of total bilirubin in arthritis AIA rats in the absence and presence of MTX and Silybin. FIG. 3F refers to the level of non-esterified fatty acids (NEFA) in arthritis AIA rats compared with normal control rats in the absence and presence of MTX and Silybin (“SB”). FIG. 3G refers to the level of total cholesterol (TC), HDL cholesterol (HDL-C) and LDL/VLDL in arthritis AIA rats compared with normal control rats in the absence and presence of MTX and Silybin. FIG. 3H refers to the TC/HDL-C ratio in arthritis AIA rats compared with normal control rats in the absence and presence of MTX and Silybin. FIG. 3I refers to the level of triglycerides (TG) in arthritis AIA rats compared with normal control rats in the absence and presence of MTX and Silybin. FIGS. 3J, 3K, 3L, and 3M show representative hematoxylin and eosin-stained sections (magnification, ×40) revealing histopathological changes in the liver of the AIA model rats. Data are expressed as means±S.E.M (n=10-11). ^{#}P<0.05, ^{##}P<0.01 ^{###}P<0.001 versus normal rats; *P<0.05, **P<0.01, ***P<0.01 versus the vehicle-treated rats.

FIGS. 4A through 4E refer to the metabolomic study of the major metabolites changed in arthritis AIA model with or without treatment. FIG. 4A shows OPLS-DA score plots based on UPLC/MS/MS spectra of plasma samples from rats in control group, arthritis AIA model without treatment (“Model”, vehicle-treated rats) or with treatment with MTX or Silybin (“SB”) in a dosage of 100 mg/kg or 200 mg/kg. 26 plasma metabolites could be identified that contribute to the discrimination of control group, model group and different treatment groups. OPLS-DA model analysis revealed an obvious separation between groups. FIGS. 4B, 4C, 4D and 4E refer to the selected metabolite set, which could be used to classify AIA model vs. control with 100% accuracy in both cohorts (changes in the relative quantities of target metabolites identified by OPLS-DA score (VIP>1.0) and student t-test (p<0.05) in different groups). FIG. 4B refers to glycochenodeoxycholic acid, taurine, LPC(16:1), LPC(22:6), LPC(20:4) and LPC(18:0). FIG. 4C refers to LPC(16:0), palmitic acid, α-tyrosine, L-kynurenine, aminohippuric acid, GSH, o-tyr/phe and citric acid. FIG. 4D refers to the GSH/GSSG ratio, succinic acid, oxaloacetate, L-leucine, xanthine, uric acid, creatine and taurochenodeoxycholic acid. FIG. 4E refers to uridine, 5-hydroxytryptamine, glycocholic acid and allantoin. ^{#}P<0.05, ^{##}P<0.01 ^{###}P<0.001 versus normal rats; *P<0.05, **P<0.01, ***P<0.01 versus the vehicle-treated rats (n=10-11).

FIGS. 5A through 5Y refer to different enzymes involved in the lipid metabolism in livers from rat AIA model with treatment with MTX or Silybin (“SB”) or without treatment (“Model”, vehicle-treated rats) by Real-time PCR and Western blot compared to normal rats (“CTR”). FIG. 5A to 5M refer to the gene expression levels of these key enzymes involved in lipid metabolism as evaluated by Real-time PCR; and FIG. 5N to 5Y refer to the protein levels as evaluated by Western blot. Real-time PCR amplification was performed for LDL, G6PD, CYP7A1, aP2, CYP27A1, CD36, SREBP1, CYP2E1, SR-B1, LDLR, CPT-1α, HMGCR, ACS, ApoC2, ApoE, FXR, LXR alpha, PPAR-alpha, PPAR-gamma, and the results were normalized for the amount of GAPDH as internal control. Protein samples were analyzed on 10% SDS-PAGE, followed by immunoblotting. The level of β-actin was determined as loading control. ^{#}P<0.05, ^{##}P<0.01 ^{###}P<0.001 versus normal rats; *P<0.05, **P<0.01, ***P<0.01 versus the vehicle-treated rats (n=6-8).

FIGS. 6A through 6M show the effects of Silybin on histopathological changes of MCD diet induced NASH mice as well as liver protection activity and anti-inflammatory activity. FIG. 6A shows representative H&E stained (×40) histopathological sections of the architecture of the liver from normal mice. FIG. 6B shows representative H&E stained (×40) histopathological sections of the architecture of the liver from vehicle-treated NASH mice. FIG. 6C shows representative H&E stained (×40) histopathological sections of the architecture of the liver from Silybin treated NASH mice at 150 mg/kg. FIG. 6D shows representative H&E stained (×40) histopathological sections of the architecture of the liver from Silybin treated NASH mice at 300 mg/kg. The MCD diet induced NASH model shows notable microvesicular steatosis, ballooning degeneration of hepatocytes and portal tract surrounded by neutrophils. FIG. 6E shows the modulating effect of Silybin and MTX on the body weight. FIG. 6F shows the modulating effect of Silybin (“SB150” 150 mg/kg or “SB300” 300 mg/kg) and MTX on the liver/body ratio in MCD diet NASH mice model after 49 days treatment. FIG. 6G shows the modulating effect of Silybin and MTX on the biomarker TC in MCD diet NASH mice model after 49 days treatment. FIG. 6H shows the modulating effect of Silybin and MTX on the biomarker TG in MCD diet NASH mice model after 49 days treatment. FIG. 6I shows the modulating effect of Silybin and MTX on the biomarker AST in MCD diet NASH mice model after 49 days treatment. FIG. 6J shows the modulating effect of Silybin and MTX on the biomarker ALT in MCD diet NASH mice model after 49 days treatment. FIG. 6K shows the modulating effect of Silybin and MTX on the biomarker TNF-α in MCD diet NASH mice model after 49 days treatment. FIG. 6L shows the modulating effect of Silybin and MTX on IL-6 in MCD diet NASH mice model after 49 days treatment. FIG. 6M shows the modulating effect of Silybin and MTX on SOD in MCD diet NASH mice model after 49 days treatment. ^{★,}* p<0.05, ^{★★,}**p<0.01, ^{★★★,}***p<0.001 versus normal rats or versus the vehicle-treated rats (n=6-8).

FIG. 7 is a schematic representation of the lipid metabolic pathways in liver of rat AIA models. The solid lines represent reactions, whereas the dotted lines indicate transport. ↑ and ↓ represent increased and decreased liver gene and/or protein expression levels, respectively, by comparing AIA model vs. normal control group. Gene symbols: acc, acetyl-Coenzyme A carboxylase; fas, fatty acid synthase; g6pd, glucose-6-phosphate dehydrogenase; lpl, lipoprotein lipase; fatp 4, fatty acid transporter protein 4.

FIGS. 8A through 8I show bar chart diagrams indicating the relative organ weight ratio including liver index (FIG. 8A), spleen index (FIG. 8B), thymus index (FIG. 8C), adrenal index (FIG. 8D), lung index (FIG. 8E), kidney index (FIG. 8F), heart index (FIG. 8G), brain index (FIG. 8H), testis index (FIG. 8I) in the control group (normal), AIA rat model (Model, vehicle-treated rats) without treatment and with treatment with MTX or Silybin in the dosage of 100 mg/kg (“SB100”) or 200 mg/kg (“SB200”) confirming that Silybin reversed changes in the relative organ weight ratio including liver index, spleen index, kidney index, lung index etc. in AIA rat model. The relative organ weight ratios (such as liver index, spleen index, kidney index, lung index) were calculated by dividing the weight of each organ by the body weight. ^{#}P<0.05, ^{##}P<0.01 ^{###}P<0.001 versus normal rats; *P<0.05, **P<0.01, ***P<0.01 versus the vehicle-treated rats (n=10-11).

FIG. 9 refers to the OPLS-DA model analysis of the metabolic profiles of plasma samples in the MCD diet induced NASH mice model with or without treatment with Silybin in the dosage of 100 mg/kg or 200 mg/kg and the control group.

FIGS. 10A through 10D refer to the metabolic patterns in the control MCS-diet fed mice (normal, “C”) and in the MCD diet induced NASH mice model (“M”) in the in the absence (“vehicle-treated rats) or presence of the treatment with Silybin in the dosage of 100 mg/kg (“SB1”) or 200 mg/kg (“5B2”). FIG. 10A refers to glycochenodeoxycholic acid, LPC(16:1), UDCA, N-phenylacetylglycine, uric acid and uridine. FIG. 10B refers to LPC(18:0), LPC(22:6), GSH, GSSG, oxaloacetate, L-kynurenine. FIG. 10C refers to LPC(20:1), trigonelline, 5-hydroxytryptamine, o-tyr/phe, proline, 4-(2-aminophenyl)-2,4-dioxobutanoic acid. FIG. 10D refers to the GSH/GSSG ratio and ophthalmic acid. ^{#}P<0.05, ^{##}P<0.01 ^{###}P<0.001 versus normal rats; *P<0.05, **P<0.01, ***P<0.01 versus the vehicle-treated rats (n=10-11).

### DETAILED DESCRIPTION OF THE INVENTION

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usually and unavoidable impurities such as side products and components usually resulting from the respective preparation or method for obtaining the material such as traces of further components or solvents. The expression that a material is certain element is to be understood for meaning “essentially consists of” said element. As used herein, the forms “a,” “an,” and “the,” are intended to include the singular and plural forms unless the context clearly indicates otherwise.

The present invention provides a method for reducing abnormalities in lipid metabolism and for reducing inflammation in a subject. Said method comprises administering an effective amount of a flavonolignan to said subject. Said subject suffers from an autoimmune inflammatory disease accompanied by abnormalities in lipid metabolism.

The term “autoimmune inflammatory disease” as used herein means diseases that are inflammatory but also autoimmune, i.e., the subject's immune system attacks itself clinically manifesting with symptoms of chronic inflammation and resulting in the simultaneous damage to body tissues. Examples of those diseases include ankylosing spondylitis, inflammatory bowel disease such as Crohn's disease or ulcerative colitis, diabetes mellitus Type 1, lupus erythematosus, multiple sclerosis, myasthenia gravis, psoriasis, psoriatic arthritis, polymyositis, dermatomyositis, vasculitis and rheumatoid arthritis.

In particular, the autoimmune inflammatory disease is an “autoimmune arthritis”, i.e. a disease in which a primary joint disorder has an autoimmune component. Due to the inflammatory nature of these diseases, they also may affect the connective tissues, soft tissues and organs. The autoimmune arthritis is in particular rheumatoid arthritis (RA), which is a chronic systemic autoimmune disease that primarily involves the joints. RA may be diagnosed depending on the species such as by means of the Disease Activity Score of 28 joints (DAS28) and/or the 2010 American College of Rheumatology (ACR)/European League Against Rheumatism (EULAR) classification criteria for RA in humans. The RA can be an active RA which can, for example, be confirmed with the DAS28 score if the subject is a human. Preferably, the disease is an early stage RA also known as “early RA”. Diagnosis criteria for early RA are known in the art.

The subject can be an animal or human, in particular a mammal such as a human. The subject is in particular a mammal such as a human with RA.

The subject is a subject having abnormalities in lipid metabolism which are in particular associated with autoimmune inflammatory disease such as RA, i.e. it is a subject with the autoimmune inflammatory disease accompanied by abnormalities in lipid metabolism as a specific patient group among patients with the autoimmune inflammatory disease. The expression “abnormalities in lipid metabolism” means a condition where abnormality occurs in the lipid and/or lipoprotein metabolism with an abnormality in levels of lipids and/or lipoproteins such as measurable in a body fluid of the subject like blood serum or blood plasma which may include lipid accumulation in the liver, in particular accompanied by abnormal levels of lipid metabolism markers, in particular polypeptides involved in the lipid and/or lipoprotein metabolism such as in the liver. The level of the lipid metabolism markers can be determined by methods known to one of skill in the art including determining its expression such as by RT-qPCR or Western blotting in cells or a tissue from the subject, in particular in liver tissue or liver cells.

The expression “associated with autoimmune inflammatory disease such as RA” means that such abnormality in lipid metabolism can be seen in a number of subjects with the autoimmune inflammatory disease such as RA and, thus, forms a specific patient group.

The term “lipids” includes triglycerides, which generally refers to any naturally occurring ester of a fatty acid and/or glycerol, and fatty acids. “Lipoproteins” are generally known as soluble proteins that combine with and transport lipids in the blood plasma and include, for example, HDL, LDL, VLDL and the like.

The abnormality in levels of lipids and/or lipoproteins can be determined in a body fluid from the subject such as blood serum or blood plasma and means a level which deviates from the reference value in healthy subjects in particular of one or more of total cholesterol (TC), high-density lipoprotein (HDL) cholesterol (HDL-C), low-density lipoprotein (LDL) cholesterol (LDL-C), free fatty acids and/or triglycerides. The abnormality can include decreased levels of HDL cholesterol with abnormalities in the levels of one or more of total cholesterol, LDL cholesterol and triglycerides such as associated with early RA.

In particular, the abnormality in levels of lipids and/or lipoproteins includes a decreased level of HDL cholesterol, in particular a significantly decreased level of HDL cholesterol compared to a reference value in healthy subjects. More preferably, the abnormality in levels of lipids and/or lipoproteins includes all of:
a decreased level of HDL cholesterol, in particular a significantly decreased level of HDL cholesterol;
a normal or increased level of total cholesterol, in particular a normal or mildly increased level of total cholesterol;
a normal or increased level of LDL cholesterol, in particular a normal or mildly increased level of LDL cholesterol; and
a normal or increased level of triglycerides, in particular a normal or mildly increased level of triglycerides;

compared to a reference value in healthy subjects.

The abnormality in levels of lipids and/or lipoproteins can further include increased levels of free fatty acids compared to a reference value in healthy subjects.

“Elevated” or “increased” level in particular means a significant increase and can, for example, further preferably mean an increase which exceeds the reference value in healthy subjects by at least 5%, in particular by at least 10%. Decrease means in particular a significant decrease and can, for example, preferably mean a decrease compared to the reference value in healthy subjects by at least 5%, in particular by at least 10%. As used herein, the term “statistically significant” means that is statistically significant as determined by Student's t-test or other art-accepted measures of statistical significance. A “mildly increased” level as used for total cholesterol, LDL cholesterol and triglycerides herein is generally an increased level with the level exceeding the reference value in healthy subjects by at most 20% to 35%. For example, a normal serum cholesterol level can be considered as a level below 200 mg/dL with a mildly increased level being between about 200 mg/dL and 240 mg/dL. A normal serum LDL cholesterol level is usually a level below 130 mg/dL with a mildly increased level being between 130 mg/dL and 159 mg/dL. A normal serum triglyceride level (fasting) is usually a level below 150 mg/dL with a mildly increased level being between 150 mg/dL and 200 mg/dL. A decreased HLD cholesterol level is, for example, a serum level below 40 mg/dL.

The level of lipid metabolism markers, in particular polypeptides involved in the lipid and/or lipoprotein metabolism is either decreased or increased, i.e. deviates from the reference value in healthy subjects, in particular it is either significantly decreased or significantly increased compared to the reference value in healthy subjects, i.e. the normal value, i.e. the concentration of lipid metabolism markers is not maintained in an appropriate range as in healthy subjects.

The term “polypeptides” which is used interchangeably with the term “protein” means a polymer of two or more amino acids connected to each other by peptide bonds between amino groups and carboxy groups of adjacent amino acid residues. The amino acid residues can be modified (e.g., phosphorylated, glycated, glycosylated, etc.). Lipid metabolism markers in particular include polypeptides with enzymatic function in the subject or polypeptides otherwise involved in the lipid metabolism such as carrier proteins for fatty acids. The expression “involved in the lipid and/or lipoprotein metabolism” means involved in the synthesis, catabolism, storage and transport of lipids and/or lipoproteins such as Lipoprotein Lipase (LPL), Adipocyte Protein 2 (aP2), Cholesterol 7-Alpha-Hydroxylase (CYP7A1), Sterol 27-Hydroxylase (CYP27A1), Glucose-6-Phosphate Dehydrogenase (G6PD), Fatty Acid Translocase (CD-36/FAT), sterol regulatory element-binding protein 1 (SREBP-1), Liver X receptor alpha (LXRalpha), Scavenger receptor class B member 1 (SR-B1), Low-Density Lipoprotein (LDL) Receptor (LDLR) and the like. Polypeptides with enzymatic function in particular include enzymes involved in the de novo synthesis, storage and transport of fatty acids or in the uptake and hydrolysis of lipoproteins.

The subject may have levels of lipid metabolism markers which deviate from the reference value in healthy subjects which can be determined in particular in liver tissue or liver cells including one, in particular at least two and most preferably or all of:
CYP7A1;
CYP27A1;
LPL;
G6PD;
aP2;
CD-36/FAT
SREBP-1;
SR-B1;
LDLR; and/or
LXRalpha.

The subject further in embodiments of the present invention suffers from a liver disease selected from one or more of a fatty liver disease such as in form of hepatic steatosis, a hepatic dysfunction or a liver fibrosis. “Hepatic steatosis” as known to one of skill in the art is a reversible condition wherein vacuoles of triglyceride fat accumulate in liver cells via the process of steatosis, i.e. abnormal retention of lipids in the cells. The term “hepatic dysfunction” means a state in which the liver function is decreased relative to healthy subjects which might be determined by means of liver function markers such as the levels of blood AST, ALT, γ-GTP, ALP, total bilirubin, albumin, LDH (lactate dehydrogenase), choline esterase and the like which are deviate from, in particular are significantly increased or decreased compared to a reference value in healthy subjects, i.e. the normal value.

In particular embodiments of the present invention, the subject further suffers from a fatty liver disease including Non-alcoholic fatty liver disease (NAFLD) or Nonalcoholic steatohepatitis (NASH).

Additionally or alternatively, the subject may further suffer from metabolic abnormalities accompanied by insulin resistance such as a metabolic syndrome, from obesity such as with a body mass index of more than 30 kg/m², from diabetes mellitus or from hypertension such as a systolic pressure of more than 140 or above and/or a diastolic pressure of 90 or above. “Metabolic syndrome” is known as a cluster of conditions including increased blood pressure, high blood sugar, excess body fat around the waist, and abnormal triglyceride levels.

“Reducing abnormalities in lipid metabolism” in particular means reducing abnormalities in lipid and/or lipoprotein levels in particular including reducing abnormalities in lipid metabolism markers such as polypeptides involved in the lipid and/or lipoprotein metabolism in particular those which are associated with the autoimmune inflammatory disease, in particular it means reducing abnormalities compared to reference values of healthy subjects. Typically, the reduction of abnormalities in lipid metabolism can be assayed by measuring the level of lipids and lipoproteins in body fluids such as blood plasma or blood serum of the subject or lipid metabolism markers, in particular polypeptides involved in the lipid and/or lipoprotein metabolism such as one, in particular two and most preferably all of LPL, G6PD, aP2, CD-36/FAT, LXRalpha, SREBP-1, CYP27A1, CYP7A1, SR-B1 and LDLR in liver tissue or liver cells.

Reducing abnormalities in levels of lipids or lipoproteins preferably includes one or more of an increase, in particular a significant increase in the level of HDL cholesterol, a decrease of triglycerides, in particular a significant decrease in the level of triglycerides and/or a decrease, in particular a significant decrease in the level of free fatty acids, in particular a significant decrease in the level of triglycerides, free fatty acids and a significant increase in the level of HDL cholesterol compared to untreated subjects with the autoimmune inflammatory disease accompanied by lipid metabolism abnormalities which can be determined in a body fluid such as blood serum or blood plasma of the subject. Further, it can include a decrease, in particular a significant decrease in the level of total cholesterol and/or LDL cholesterol.

Reducing abnormalities in lipid metabolism markers can include decreasing one or more elevated lipid metabolism markers compared to untreated subjects having the autoimmune inflammatory disease accompanied by lipid metabolism abnormalities, in particular significantly decreasing one, two or all of:
elevated CYP7A1, in particular an at least about 10% decrease, further preferred more than about 20% and still further preferred more than about 40%;
elevated LPL, in particular an at least about 10% decrease, further preferred more than about 20%;
elevated G6PD, in particular an at least about 10% decrease, further preferred more than about 20%;
elevated aP2, in particular an at least about 10% decrease, further preferred more than about 20% and still further preferred more than about 40%;
elevated CD-36/FAT, in particular an at least about 10% decrease, further preferred more than about 20%;
elevated CYP27A1, in particular an at least about 10% decrease, further preferred more than about 20%;
elevated LXRalpha, in particular an at least about 10% decrease, further preferred more than about 20%;
elevated SR-B1, in particular an at least about 10% decrease, further preferred more than about 20%;
elevated LDLR, in particular an at least about 10% decrease.

“Reducing inflammation” means reducing abnormal inflammatory markers which, in particular, is accompanied by one or more of a reduced synovial inflammation, reduced swelling and/or a reduced joint and cartilage damage. “Inflammatory markers” are those which usually indicate an inflammation in the subject. They can be determined with methods known to one of skill in the art such as in cells, tissues or body fluids from the subject, in particular in the blood serum or blood plasma of the subject. Inflammatory markers in particular include Tumor Necrosis Factor-alpha (TNF-α), Interleukin-1β (IL-1β), Prostaglandin E2 (PGE2), Matrix Metallopeptidase 9 (MMP-9), TIMP Metalloproteinase Inhibitor 1 (TIMP-1), Interleukin 17 (IL-17) and the Erythrocyte Sedimentation Rate (ESR) and the like. More preferably, reducing inflammation means decreasing elevated inflammatory markers, in particular significantly decreasing elevated inflammatory markers compared to untreated subjects with the autoimmune inflammatory disease such as selected from one, in particular two and most preferably all of TNF-α, IL-1β, PGE2, MMP-9, TIMP-1 or ESR most preferably to reference values as in healthy subjects. The level of inflammatory markers can be determined, for example, by means of commercially available kits. A reduced synovial inflammation, swelling and/or a reduced joint and cartilage damage can be confirmed by determining cartilage and bone erosions with X-ray or MRI, by means of arthritis indices such as ACR 20, 50, or 70, or a disease activity score of DAS 28 or some radiographic outcome, in particular a significant improvement in the DAS28 score may indicate a reduced inflammation.

In particular, reducing inflammation means a decrease, preferably a significant decrease in TNF-α, IL-1β, PGE2, MMP-9, TIMP-1 levels and/or ESR which can be determined in a body fluid such as blood serum or blood plasma from the subject, further preferably one, two or most preferably all of:
a decrease in ESR by at least 10%, in particular at least 20% compared to untreated subjects having the autoimmune inflammatory disease;
a decrease in IL-1β of at least 5%, in particular more than 10% compared to untreated subjects having the autoimmune inflammatory disease;
a decrease in TNF-α of at least 5%, in particular more than 10% compared to untreated subjects having the autoimmune inflammatory disease;
a decrease in MMP-9 of at least 5%, in particular more than 10% compared to untreated subjects having the autoimmune inflammatory disease;
a decrease in TIMP-1 of 5%, in particular more than 10% compared to untreated subjects having the autoimmune inflammatory disease;
a decrease in PGE2 of 5%, in particular more than 10% and further preferred of more than 20% compared to untreated subjects having the autoimmune inflammatory disease.

The flavonolignan is in particular a lipid-modulating and hepatoprotective flavonolignan, i.e. the flavonolignan is a lipid-modulator with hepatoprotective function and, thus, improves liver function with modulation of lipid metabolism. As used herein, the term “lipid modulator” means that the compound is able to alter lipid metabolism and storage in the subject. The term “hepatoprotective” is intended to mean that the compound is also able to prevent damage to the liver, i.e. is able to protect liver cells. The compound can, for example, be able to inhibit free radicals that are produced from the metabolism of hepatotoxic substances, to enhance hepatic glutathione, to contribute to the antioxidant defense of the liver and/or to increases protein synthesis in hepatocytes. Typically, the hepatoprotective effect can be assayed in the presence of hepatotoxic compounds such as ethanol. In particular, the flavonolignan is able to reduce abnormalities in levels of Alkaline Phosphatase (ALP) and/or Aspartate Aminotransferase (AST), in particular to reduce and further preferred significantly reduce elevated ALP and AST levels such as an at least 5% and further preferred more than about 10% decrease compared to untreated subjects having the autoimmune inflammatory disease and hepatic abnormalities which can be determined with commercially available kits in body fluids such as plasma or serum from the subject.

The subject may further have hepatic abnormalities including an increased level of Alkaline Phosphatase (ALP) and/or Aspartate Aminotransferase (AST) compared to a reference value in healthy subjects which can be determined in body fluids from the subject, in particular the hepatic abnormalities are associated with the autoimmune inflammatory disease. The method in such embodiments further comprises reducing hepatic abnormalities in particular including reducing, in particular significantly reducing the level of ALP and/or ASP, in particular of both such as by at least about 5%, in particular by at least about 20% compared to untreated subjects suffering from an autoimmune inflammatory disease such as RA and the hepatic abnormalities.

In particular embodiments of the present invention, the subject further has a fatty liver disease such as in form of a hepatic steatosis and the flavonolignan in particular further reduces accumulation of lipids in the liver, i.e. the method is in embodiments of the present invention further for reducing accumulation of lipids in the liver.

The method can in further embodiments include reducing the risk of a cardiovascular disease such as ischemic heart disease, heart failure, myocardial infarction, stroke or sudden cardiac death.

The flavonolignan can be a single flavonolignan or a mixture of flavonolignans. The term flavonolignan as known to one of skill in the art is generally used for compounds in which coniferyl alcohol is coupled to the flavanone basic structure, i.e.:

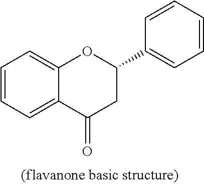 [SMILES: O=C1C[C@@H](c2ccccc2)Oc2ccccc21]

at different positions. Namely coniferyl alcohol

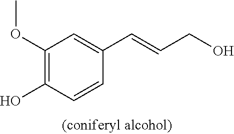

is coupled to a 3-hydroxyflavanone also known as taxifolin at different positions:

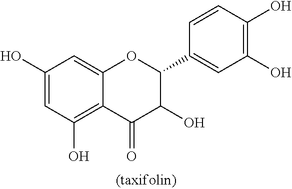 [SMILES: O=C1c2c(O)cc(O)cc2O[C@H](c2ccc(O)c(O)c2)C1O]

Flavonolignans include, for example, structures of Formula (I) to (IV) or glycosides, salts or solvates thereof:

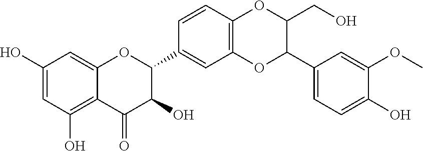 [SMILES: COc1cc(C2Oc3cc([C@H]4Oc5cc(O)cc(O)c5C(=O)[C@@H]4O)ccc3OC2CO)ccc1O]

Formula (I) including stereoisomers and mixtures thereof, in particular Silybin A and Silybin B;

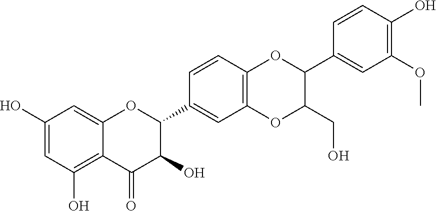 [SMILES: COc1cc(C2Oc3ccc([C@H]4Oc5cc(O)cc(O)c5C(=O)[C@@H]4O)cc3OC2CO)ccc1O]

Formula (II) including stereoisomers and mixtures thereof, in particular Isosilybin A and Isosilybin B;

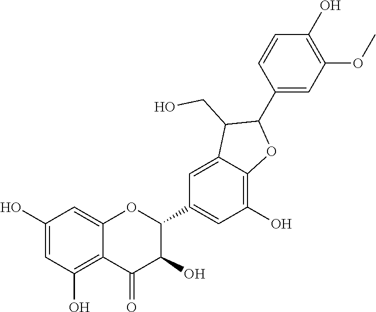 [SMILES: COc1cc(C2Oc3c(O)cc([C@H]4Oc5cc(O)cc(O)c5C(=O)[C@@H]4O)cc3C2CO)ccc1O]

Formula (III) including stereoisomers and mixtures thereof, in particular Silychristin;

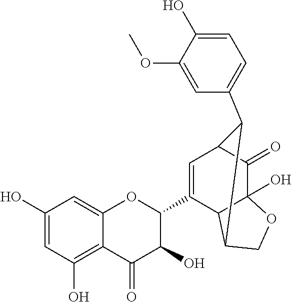 [SMILES: COc1cc(C2C3C=C([C@H]4Oc5cc(O)cc(O)c5C(=O)[C@@H]4O)C4C2COC4(O)C3=O)ccc1O]

Formula (IV) including stereoisomers and mixtures thereof, in particular Silydianin.

The flavonolignan in particular includes one or more compounds of Formula (Ia), (Ib), (IIa), (IIb), (IIIa) and/or (IVa) or glycosides, salts or solvates thereof:

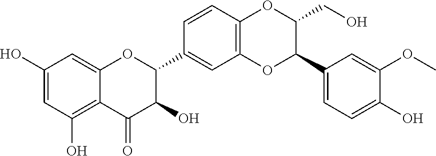 [SMILES: COc1cc([C@H]2Oc3cc([C@H]4Oc5cc(O)cc(O)c5C(=O)[C@@H]4O)ccc3O[C@@H]2CO)ccc1O]

Formula (Ia), i.e. Silybin A;

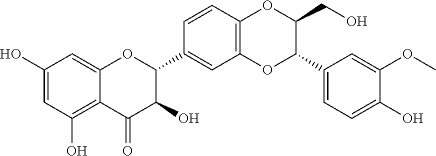 [SMILES: COc1cc([C@@H]2Oc3cc([C@H]4Oc5cc(O)cc(O)c5C(=O)[C@@H]4O)ccc3O[C@H]2CO)ccc1O]

Formula (Ib), i.e. Silybin B;

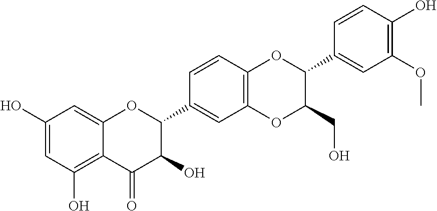 [SMILES: COc1cc([C@H]2Oc3ccc([C@H]4Oc5cc(O)cc(O)c5C(=O)[C@@H]4O)cc3O[C@@H]2CO)ccc1O]

Formula (IIa), i.e. Isosilybin A;

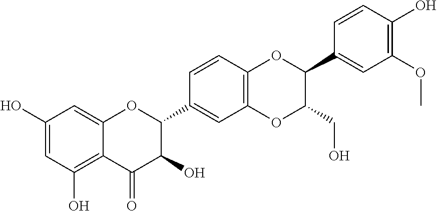 [SMILES: COc1cc([C@@H]2Oc3ccc([C@H]4Oc5cc(O)cc(O)c5C(=O)[C@@H]4O)cc3O[C@H]2CO)ccc1O]

Formula (IIb), i.e. Isosilybin B;

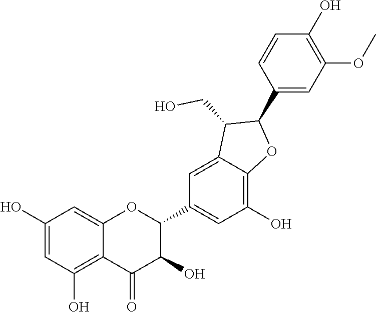 [SMILES: COc1cc([C@H]2Oc3c(O)cc([C@H]4Oc5cc(O)cc(O)c5C(=O)[C@@H]4O)cc3[C@@H]2CO)ccc1O]

Formula (IIIa), i.e. Silychristin;

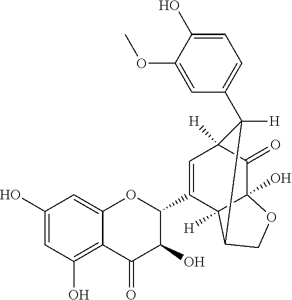 [SMILES: COc1cc(C2C3CO[C@]4(O)C(=O)[C@H]2C=C([C@H]2Oc5cc(O)cc(O)c5C(=O)[C@@H]2O)[C@H]34)ccc1O]

Formula (IV), i.e. Silydianin.

Also contemplated by the present invention are any salts, solvates as well as stereoisomers and their mixtures of the compounds given above. Stereoisomers are isomers with the same order of the atoms but different 3D arrangement of the atoms and include diastereomers and optical isomers (enantiomers). As used herein, the term “solvate” refers to a complex of variable stoichiometry formed by a solute, i.e. the flavonolignan, and a solvent. If the solvent is water, the solvate formed is a hydrate. Suitable salts are those which are suitable to be administered to subjects, in particular mammals such as humans and can be prepared with sufficient purity.

The flavonolignan of the present invention in particular comprises a compound of Formula (I) or a glycoside, salt or solvate thereof including the stereoisomers of Formula (Ia) and/or (Ib), i.e. the flavonolignan of the present invention more preferably comprises Silybin A and/or Silybin B including glycosides, salts or solvates thereof and optionally further flavonolignans. Further flavonolignans may in particular comprise compounds of Formula (II), (III) and/or (IV) such as (IIa), (IIb), (IIIa) and/or (IVa). For example, Silymarin can be used as flavonolignan of the present invention comprising compounds of Formula (Ia), (Ib), (IIa), (IIb), (IIIa) and (IVa). In most preferred embodiments, the flavonolignan of the present invention is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both, especially preferably a mixture of both such as a 50:50 (w/w) mixture of Silybin A and Silybin B.

The flavonolignan of the present invention, which is in particular a mixture of Silybin A and Silybin B can be obtained from Silybum marianum by an extraction or is commercially available. The skilled person is aware of suitable extraction methods for obtaining a flavonolignan from S. marianum.

The expression “effective amount” generally denotes an amount sufficient to produce therapeutically desirable results, wherein the exact nature of the result varies depending on the specific disorder which is treated. In the present invention, it means an amount of the flavonolignan at least able to reduce abnormalities in lipid metabolism and reduce inflammation in the subject suffering from the autoimmune arthritis, namely able to reduce, more preferably significantly reduce abnormalities in lipid metabolism markers and inflammation markers. The effective amount may depend on the species, body weight, age and individual conditions of the subject and can be determined by standard procedures such as with cell cultures or experimental animals. For instance, the effective amount of the flavonolignan of the present invention may be between about 0.5 mg/kg and 500 mg/kg body weight per day such as, for example, about 200 mg/kg body weight. The flavonolignan can be present in solid, semisolid or liquid form to be administered by an oral or parenteral route to a subject, preferably by an oral route.

The treatment may be carried out for at least about 12 days, in particular for at least about 42 days.

The flavonolignan may be administered in form of a pharmaceutical composition comprising the flavonolignan and a pharmaceutically tolerable excipient such as selected from a pharmaceutically acceptable carrier, salt, buffer, water, diluent, a filler, a binder, a disintegrant, a lubricant, a coloring agent, a surfactant or a preservative or a combination thereof.

The skilled person is able to select suitable pharmaceutically tolerable excipients depending on the form of the pharmaceutical composition and is aware of methods for manufacturing pharmaceutical compositions as well as able to select a suitable method for preparing the pharmaceutical composition depending on the kind of pharmaceutically tolerable excipients and the form of the pharmaceutical composition. The pharmaceutical composition can be present in solid, semisolid or liquid form to be administered by an oral or parenteral route to a subject, preferably by an oral route.

The flavonolignan may be administered in combination with other compounds for treating autoimmune inflammatory diseases such as nonsteroidal anti-inflammatory drugs (NSAIDs) or disease-modifying antirheumatic drugs (DMARD) such as methotrexate (MTX) or leflunomide.

The present invention further provides a method for reducing the risk of a cardiovascular disease in a subject comprising administering an effective amount of a flavonolignan to said subject, wherein the subject suffers from an autoimmune inflammatory disease accompanied by abnormalities in lipid metabolism.

The subject in particular has an elevated ESR, in particular a significantly increased ESR.

The abnormalities in lipid metabolism in particular include a level which deviates from the reference value in healthy subjects in particular of one or more of total cholesterol (TC), high-density lipoprotein (HDL) cholesterol (HDL-C), low-density lipoprotein (LDL) cholesterol (LDL-C), free fatty acids and/or triglycerides.

The abnormalities in lipid metabolism preferably include a decreased level of HDL cholesterol, in particular a significantly decreased level of HDL cholesterol compared to a reference value in healthy subjects. More preferably, the abnormality in levels of lipids and/or lipoproteins includes all of:
a decreased level of HDL cholesterol, in particular a significantly decreased level of HDL cholesterol;
a normal or increased level of total cholesterol, in particular a normal or mildly increased level of total cholesterol;
a normal or increased level of LDL cholesterol, in particular a normal or mildly increased level of LDL cholesterol; and
a normal or increased level of triglycerides, in particular a normal or mildly increased level of triglycerides;

compared to a reference value in healthy subjects.

The abnormality in levels of lipids and/or lipoproteins can further include increased levels of free fatty acids compared to a reference value in healthy subjects.

In alternative embodiments, the abnormalities in lipid metabolism may include a decreased level of HDL cholesterol, of total cholesterol and of LDL cholesterol, in particular a significantly decreased level of HDL cholesterol, total cholesterol and of LDL cholesterol compared to a reference value in healthy subjects. The abnormalities can further include increased levels of free fatty acids and/or triglycerides, in particular a significant increase in the level of free fatty acids and/or triglycerides compared to a reference value in healthy subjects.

The method in particular comprises reducing the risk of ischemic heart disease including angina, heart failure, of myocardial infarction, stroke and/or sudden cardiac death due to the reduction in abnormalities in lipid metabolism and reduction in inflammation by administering the effective amount of the flavonolignan.

The flavonolignan is in particular a lipid-modulating and hepatoprotective flavonolignan. The disease is in particular an autoimmune arthritis, in particular RA and the subject is preferably a mammal such as a human. The subject may further have one or more of hypertension, obesity, diabetes mellitus or metabolic syndrome, in particular the subject has a metabolic syndrome.

The flavonolignan can be a single flavonolignan or a mixture of flavonolignans. The flavonolignan in particular comprises a compound of Formula (I) or a glycoside, salt or solvate thereof including the stereoisomers of Formula (Ia) and/or (Ib). I.e. the flavonolignan of the present invention more preferably comprises Silybin A and/or Silybin B, i.e. compounds of Formula (Ia) and (Ib):

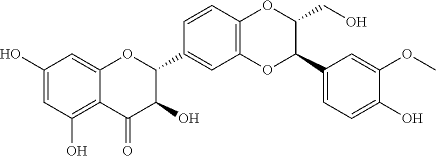 [SMILES: COc1cc([C@H]2Oc3cc([C@H]4Oc5cc(O)cc(O)c5C(=O)[C@@H]4O)ccc3O[C@@H]2CO)ccc1O]

Silybin A which is known for having Formula (Ia); and/or

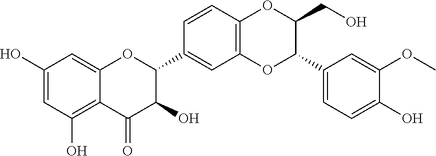 [SMILES: COc1cc([C@@H]2Oc3cc([C@H]4Oc5cc(O)cc(O)c5C(=O)[C@@H]4O)ccc3O[C@H]2CO)ccc1O]

Silybin B which is known for having Formula (Ib);

and optionally further flavonolignans in particular comprising compounds of Formula (II), (III) and/or (IV) such as (IIa), (IIb), (IIIa) and/or (IVa):

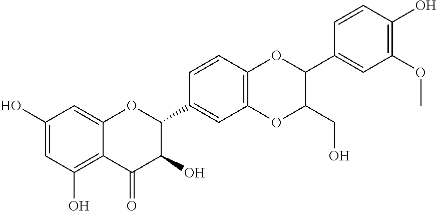 [SMILES: COc1cc(C2Oc3ccc([C@H]4Oc5cc(O)cc(O)c5C(=O)[C@@H]4O)cc3OC2CO)ccc1O]

Formula (II) such as Isosilybin A and Isosilybin B, i.e. compounds of Formula (IIa) and/or (IIb):

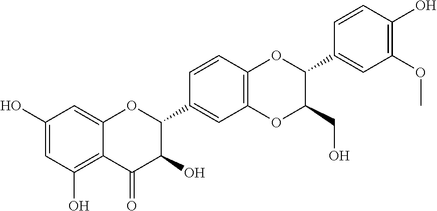 [SMILES: COc1cc([C@H]2Oc3ccc([C@H]4Oc5cc(O)cc(O)c5C(=O)[C@@H]4O)cc3O[C@@H]2CO)ccc1O]

Formula (IIa), i.e. Isosilybin A;

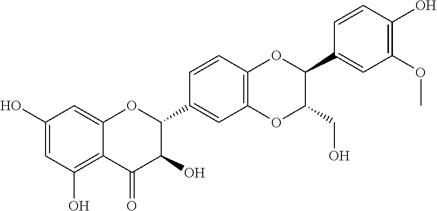 [SMILES: COc1cc([C@@H]2Oc3ccc([C@H]4Oc5cc(O)cc(O)c5C(=O)[C@@H]4O)cc3O[C@H]2CO)ccc1O]

Formula (IIb), i.e. Isosilybin B;

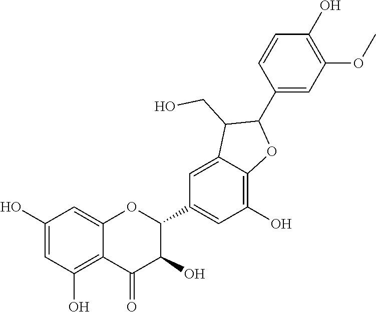 [SMILES: COc1cc(C2Oc3c(O)cc([C@H]4Oc5cc(O)cc(O)c5C(=O)[C@@H]4O)cc3C2CO)ccc1O]

Formula (III) including stereoisomers and mixtures thereof, in particular Silychristin, i.e. a compound of Formula (IIIa):

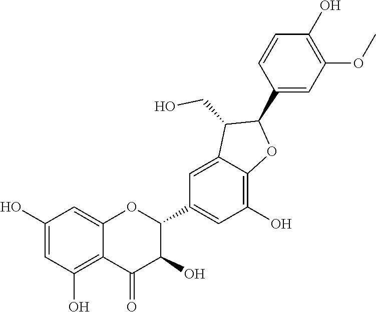 [SMILES: COc1cc([C@H]2Oc3c(O)cc([C@H]4Oc5cc(O)cc(O)c5C(=O)[C@@H]4O)cc3[C@@H]2CO)ccc1O]

Formula (IIIa);

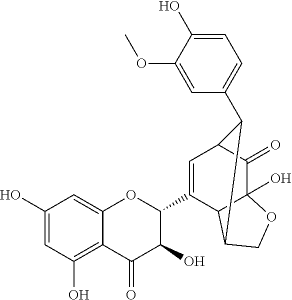 [SMILES: COc1cc(C2C3C=C([C@H]4Oc5cc(O)cc(O)c5C(=O)[C@@H]4O)C4C2COC4(O)C3=O)ccc1O]

Formula (IV) including stereoisomers and mixtures thereof, in particular Silydianin, i.e. a compound of Formula (IVa):

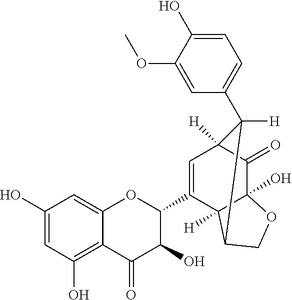 [SMILES: COc1cc(C2C3CO[C@]4(O)C(=O)[C@H]2C=C([C@H]2Oc5cc(O)cc(O)c5C(=O)[C@@H]2O)[C@H]34)ccc1O]

Formula (IVa);

or glycosides, salts or solvates of the above compounds.

For example, Silymarin can be used as flavonolignan. In most preferred embodiments, the flavonolignan of the present invention is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both, especially preferably a mixture of both such as a 50:50 (w/w) mixture of Silybin A and Silybin B.

The effective amount of the flavonolignan may be between about 0.5 mg/kg and about 500 mg/kg body weight per day such as, for example, about 200 mg/kg body weight. The flavonolignan can be present in solid, semisolid or liquid form to be administered by an oral or parenteral route to a subject, preferably by an oral route for at least about 12 days, in particular for at least about 42 days. The flavonolignan may be administered in form of a pharmaceutical composition.

Still further provided by the present invention is a method for reducing hepatic abnormalities and reducing inflammation in a subject. Said method comprises administering an effective amount of a combination of a flavonolignan and a disease-modifying antirheumatic drug to said subject. Said subject suffers from an autoimmune inflammatory disease accompanied by hepatic abnormalities and in particular additionally by lipid metabolism abnormalities.

The flavonolignan is in particular a lipid-modulating and hepatoprotective flavonolignan. The disease is in particular an autoimmune arthritis, in particular RA and the subject is preferably a mammal such as a human.

The flavonolignan can be a single flavonolignan or a mixture of flavonolignans. The flavonolignan in particular comprises a compound of Formula (I) or a glycoside, salt or solvate thereof including the stereoisomers of Formula (Ia) and/or (Ib). I.e. the flavonolignan of the present invention more preferably comprises Silybin A and/or Silybin B

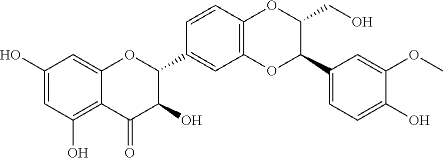 [SMILES: COc1cc([C@H]2Oc3cc([C@H]4Oc5cc(O)cc(O)c5C(=O)[C@@H]4O)ccc3O[C@@H]2CO)ccc1O]

Formula (Ia) which is Silybin A; and/or

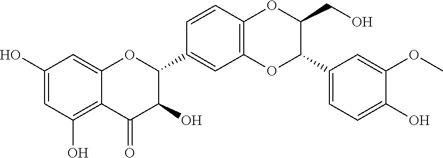 [SMILES: COc1cc([C@@H]2Oc3cc([C@H]4Oc5cc(O)cc(O)c5C(=O)[C@@H]4O)ccc3O[C@H]2CO)ccc1O]

Formula (Ib) which is Silybin B;

and optionally further flavonolignans in particular comprising compounds of Formula (II), (III) and/or (IV) such as (IIa), (IIb), (IIIa) and/or (IVa):

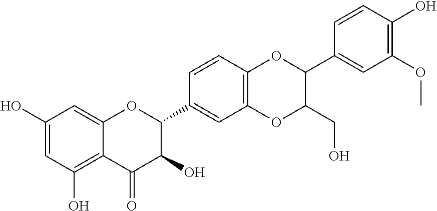 [SMILES: COc1cc(C2Oc3ccc([C@H]4Oc5cc(O)cc(O)c5C(=O)[C@@H]4O)cc3OC2CO)ccc1O]

Formula (II) such as Isosilybin A and Isosilybin B, i.e. compounds of Formula (IIa) and/or (IIb):

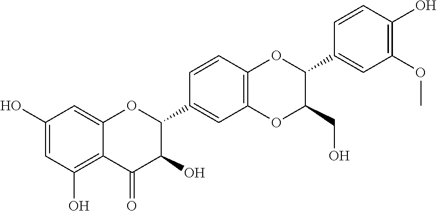 [SMILES: COc1cc([C@H]2Oc3ccc([C@H]4Oc5cc(O)cc(O)c5C(=O)[C@@H]4O)cc3O[C@@H]2CO)ccc1O]

Formula (IIa), i.e. Isosilybin A;

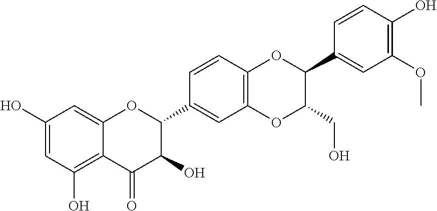 [SMILES: COc1cc([C@@H]2Oc3ccc([C@H]4Oc5cc(O)cc(O)c5C(=O)[C@@H]4O)cc3O[C@H]2CO)ccc1O]

Formula (IIb), i.e. Isosilybin B;

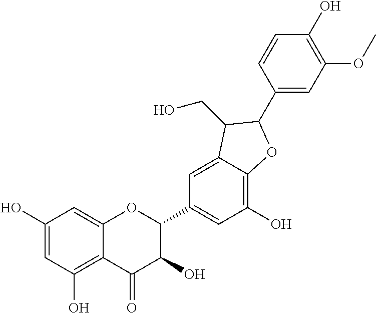 [SMILES: COc1cc(C2Oc3c(O)cc([C@H]4Oc5cc(O)cc(O)c5C(=O)[C@@H]4O)cc3C2CO)ccc1O]

Formula (III) including stereoisomers and mixtures thereof, in particular Silychristin, i.e. a compound of Formula (IIIa):

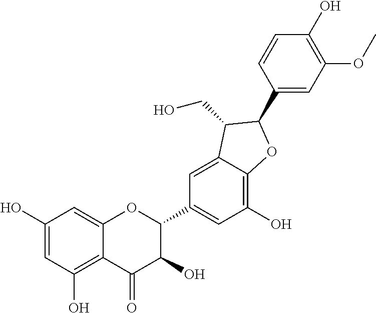 [SMILES: COc1cc([C@H]2Oc3c(O)cc([C@H]4Oc5cc(O)cc(O)c5C(=O)[C@@H]4O)cc3[C@@H]2CO)ccc1O]

Formula (IIIa);

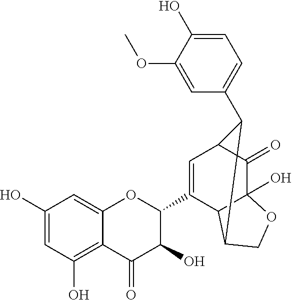 [SMILES: COc1cc(C2C3C=C([C@H]4Oc5cc(O)cc(O)c5C(=O)[C@@H]4O)C4C2COC4(O)C3=O)ccc1O]

Formula (IV) including stereoisomers and mixtures thereof, in particular Silydianin, i.e. a compound of Formula (IVa):

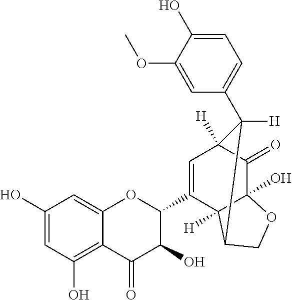 [SMILES: COc1cc(C2C3CO[C@]4(O)C(=O)[C@H]2C=C([C@H]2Oc5cc(O)cc(O)c5C(=O)[C@@H]2O)[C@H]34)ccc1O]

Formula (IVa);

or glycosides, salts or solvates of the above compounds.

For example, Silymarin can be used as flavonolignan. In most preferred embodiments, the flavonolignan of the present invention is:
Silybin A (i.e. a compound of Formula (Ia)) or a glycoside, salt or solvate thereof;
Silybin B (i.e. a compound of Formula (Ib)) or a glycoside, salt or solvate thereof;

or a mixture of both, especially preferably a mixture of both such as a 50:50 (w/w) mixture of Silybin A and Silybin B.

The effective amount of the flavonolignan may be between about 0.5 mg/kg and about 500 mg/kg body weight per day such as, for example, about 200 mg/kg body weight. The flavonolignan can be present in solid, semisolid or liquid form to be administered by an oral or parenteral route to a subject, preferably by an oral route for at least about 12 days, in particular for at least about 42 days. The flavonolignan may be administered in form of a pharmaceutical composition.

Disease-modifying antirheumatic drugs (DMARDs) is a group of drugs commonly used for treatment of autoimmune arthritis such as RA and known to one of skill in the art for improving symptoms, decreasing joint damage, and improving overall functional abilities. They in particular include methotrexate, hydroxychloroquine, sulfasalazine, leflunomide, TNF-alpha inhibitors (certolizumab, infliximab and etanercept), abatacept, and anakinra, rituximab and tocilizumab. The DMARD used in combination with the flavonolignan is in particular methotrexate (MTX) such as in a dose of 1 mg to 100 mg MTX such as 1 mg to 50 mg MTX per day or, for example, 0.1 mg/kg to 10 mg/kg MTX per body weight per day depending on the species, body weight, age and individual conditions of the subject. The flavonolignan and MTX are in particular administered by an oral route.

The hepatic abnormality in particular includes increased levels of Alkaline Phosphatase (ALP) and/or Aspartate Aminotransferase (AST) compared to a reference value in healthy subjects which can be determined in body fluids from the subject and are in particular associated with the autoimmune inflammatory disease and/or with the DMARD treatment, in particular associated with the autoimmune inflammatory disease and with the DMARD such as MTX treatment.

Reducing hepatic abnormalities in such embodiments in particular includes reducing, in particular significantly reducing the level of ALP and/or ASP, in particular of both such as by at least about 5%, in particular by at least about 20% compared to untreated subjects suffering from an autoimmune inflammatory disease such as RA accompanied by hepatic abnormalities.

Still further provided with the present invention is a method for reducing liver damages due to the treatment of rheumatoid arthritis with a DMARD or a NSAID. Said method comprises administering an effective amount of a flavonolignan to said subject before, after or simultaneously with the DMARD or NSAID, in particular simultaneously with the DMARD.

### EXAMPLES

### Chemicals and Reagents

A 50:50 (w/w) mixture of Silybin A and Silybin B, referenced as “Silybin” hereinafter and tangeretin were purchased from Meryer (Meryer Chermical Technology Co., Ltd., ShangHai, China); glutathione (GSH), L-leucine, L-kynurenine, L-tryptophan, 5-hydroxytryptophan (5-HTP), cholic acid, N-phenylacetylglycine, 5-hydroxytryptamine (5-HT) were purchased from Melonepharma (Dalian meilune Biology Technology Co., Ltd., DaLian, China); N-ethylmaleimide (≥98%, HPLC), L-glutathione oxidized (GSSG, ≥98%, HPLC) were purchased from Sigma (Sigma-Aldrich, Co. Ltd., St. Louis, USA); L-phenylalanine (Ring-D5, 98%, DLM-1258-5) was purchased from Cambridge Isotope Laboratories (Cambridge Isotope Laboratories, Inc., MA, USA); Polyethylene glycol 400 (PEG400) was purchased from TCI (Tokyo chemical industry Co., Ltd., Tokyo, Japan); Cremophor EL (polyoxyethylene castor oil) was purchased from Aladdin (Aladdin Industrial Corporation, Shanghai, China); Menthol and acetonitrile was purchased from ACS (Anaqua chemicals supply, Houston, USA). Primary antibodies used include LPL, PPAR alpha, CYP7A1, FXR, LXR, AP2/FABP4, CYP27A1, CD36, CYP2E1 from Abcam (Abcam, Cambridge, UK), G6PD from Danvers (Danvers, Mass., USA), and SRBP1 from Santa Cruz (Santa Cruz, Calif., USA). TNF-α, interleukin (IL)-1β, and TIMP-1 ELISA kit were purchased from RayBiotech, Inc., Norcross, Ga., USA; IL-6, IL-17, IL-33, matrix metalloproteinase 9 (MMP-9), and prostaglandin E2 (PGE2) ELISA kit from Bio-techne, MN, USA; high-density lipoprotein cholesterol (HDL-C) from Bioassay, Hayward, Calif., USA. Serum free fatty acid, alanine aminotransferase (ALT), aspartate aminotransferase (AST), triglycerides (TG), total cholesterol (TC), superoxide dismutase (SOD) assay kit and total cholesterol quantification kit was purchased from NanJing JianCheng Bioengineering Institute, NanJing, China.

Animals

Experiments with the AIA model were performed on male Sprague-Dawley (SD) rats (weight 180-220 gm), and MCD diet induced NASH experiments were carried out with male wild-type (WT) C57Bl/6 mice (Guangdong Medical Laboratory Animal Center). All animals were kept in a temperature-controlled room at a constant temperature of 24±1° C. (mean±SEM) and with a 12-hour light/dark cycle. Food and water were provided ad libitum. All procedures involving animals and their care were approved and under the regulations of the animal care and use committee at Guangzhou University of Chinese Medicine.

Statistical Analysis

Data are expressed as mean±S.E.M. Differences among means were analyzed using one-way ANOVA after Gaussian distribution evaluation by a Kolmogorov-Smirnov test. The Tukey-Kramer multiple comparison test for all pairs of columns was applied as a post hoc test. In all instances, P<0.05 was taken as the lowest level of significance. GraphPad Prism 4 for Windows (GraphPad Software Inc., San Diego, Calif.) was used to perform all of the statistical analysis.

### Example 1A

Induction of AIA

The AIA model was induced on day 0 by a single injection of 0.1 ml of a freshly prepared ground Mycobacterium tuberculosis (MT) H37Ra (BD, Sparks, USA) suspension containing 62.5 μg MT at the base of the tail of animals through subcutaneous routes (Cai, X., et al., Naunyn Schmiedebergs Arch Pharmacol, 2006, 373(2): p. 140-7). Rats in the control groups were injected with an equal volume of saline instead of MT suspension. AIA rats (n=10-12) were daily treated orally with Silybin with a dosage of 100 mg/kg and 200 mg/kg (dissolve with 20% PEG400: 15% Cremophor EL: 5% ethanol: 60% saline), or MTX (Sigma, St. Louis, Mont.) with a dose of 7.6 mg/kg or vehicle (20% PEG400: 15% Cremophor EL: 5% ethanol: 60% saline) throughout the 42-day experiment.

Assessments of the Arthritis Severity and the Effects of Silybin Treatments

Disease severity and progression were evaluated by measurements of both hind paw volumes with a plethysmometer chamber (Yiyan technology, Jinan, China) and body weight with a 0.1 g precision balance (Sartorius AG, Göttingen, Germany) every two or three days after arthritis induction. For evaluation the arthritic scoring, the lesions of four paws of each rat (i.e., the arthritic signs) were graded by two separate investigators with a semi-quantitative scale: 0 (normal), 4-8 (mild changes), 8-12 (moderate changes), and 12-16 (severe changes) (van Eden W., et. al., J. E. Coligan, Editor. 2001, John Wiley: New York. p. 1-11). Erythrocyte sedimentation rate (ESR) was determined with ICSH (International Council for Standardization in Hematology) selected method with little modifications at day 42. Briefly, 120 μl of blood sample was taken directly and put into 30 μl of 0.109 mol/L sodium citrate, mixed well, and then transferred into a 1.0 mm×100 mm capillary tube (West China Medical University, ChengDu, China). The tubes were held obliquely at an angle of 45° C. and the erythrocyte sedimentation rate was recorded at 15 min. The cytokines levels including IL-1β, TNF-α, IL-17, IL-33, MMP-9, TIMP-1 and PGE2 in serum were measured using commercially available ELISA kits according to the manufacturers instructions. At the end of the experiments, rat knee images were measured (mean of three readings per knee, Meinaite digital calliper, ShangHai, China) using a IVIS Lumina XRMS Series (PerkinElmer, America) (Esser, R. E., et al., Arthritis Rheum, 1995, 38(1): p. 129-38).

For histopathological assessment, arthritic paws were collected on day 42 after X-ray check, fixed with 10% phosphate buffered saline (PBS) buffered formalin, and decalcified for 3 days in formic acid. Sections from paraffin-embedded tissue were stained with hematoxylin and eosin (H&E). The obtained plasma was stored in −70° C. for metabolomic studies. The relative organ weight ratios (such as liver index, spleen index, kidney index, lung index) were calculated by dividing the weight of each organ with the body weight.

Assessment of Liver Abnormality in AIA Model

Serum ALT, AST, ALP, GGT and total bilirubin were measured by automatic biochemical analyzer (Toshiba Corporation, Tokyo, Japan). Lipids profile including TG, TC, HDL-C, LDL-C/vLDL-C and free fatty acids were measured in the serum. After animals were sacrificed, tissues of liver were fixed in 10% formalin solution, cleared in xylene, embedded in paraffin blocks from which 5 micron-thick sections were obtained. Sections were stained with H&E dye and examined for pathological changes under light microscope by a pathologist blind to the specimens.

Metabolomics Study by LC-MS/MS

The plasma sample (200 μL) was thawed on ice for 5 min prior to sample preparation and was mixed with 200 μL of 10 mM N-ethylmaleimide (NEM) in PBS buffer solution and 1000 μL of methanol containing internal standard (I.S.) Phe-d5 in 10 ng/ml. This mixture was incubated at −20° C. for 20 min, and then centrifuged at 12000 rpm for 10 min at 4° C. The supernatant was completely dried under nitrogen flow (Pressure Blowing Concentrator, Tokyo Rikakika, Tokyo, Japan) and reconstituted with distilled water.

LC-MS/MS analysis was performed on A Waters ACQUITY UPLC coupled with a 4000 Q-TRAP mass spectrometer. Chromatographic separation was carried on a Waters X Bridge™ BEH C18 analytical column (2.5 μm, 3.0×100 mm; Waters, Torrance, Calif.) with a mobile phase composed of 0.1% formic acid water (solvent A) and methanol (solvent B) which was running in a gradient program: 0-3.0 min (0%-1% B); 3.0-10.0 min (1-3% B); 10.0-14.0 min (3-50% B); 14.0-18.0 min (50-95% B); 18.0-22.0 min (95-0% B); followed by a 3-min re-equilibration step. The injection volume was 10 μl at 4° C. and flow rate was 0.6 ml/min. Electrospray (ESI) source were used in both positive and negative ion mode. The source parameters were set as follows: gas temperature: 450° C.; the ion spray voltage, ±4500 V; ion source gas 1 (nebulizer gas) 40 psi (N2); ion source gas 2 (auxiliary gas), 40 psi (N2); curtain gas: 20 psi. The MS/MS analysis was acquired in targeted MS/MS (MRM) mode with the collision energy ranging from 10 V to 40 V. All UPLC-MS data were obtained by AB Analyst Software (Version 1.6.2). Table 1 lists the 72 metabolites that are identified in the plasma samples from both AIA and MCD-diet induced NASH animal studies. The intensity of each ion was normalized with respect to peak area of I.S. for each chromatogram prior to multivariate statistical analysis.

TABLE 1
Information of biomarkers determined by the LC-MS/MS method

| | Biomarker | Q1Mass(Da) | Q3Mass(Da) | Retention time (min) |
|---|---|---|---|---|
| 1 | Uric acid | 169.1 | 169.1 | 1.49 |
| 2 | Uridine | 243.1 | 243.1 | 16.33 |
| 3 | Allantoin | 157.1 | 114 | 0.83 |
| 4 | Xanthosine | 285 | 153 | 6.48 |
| 5 | Xanthine | 153 | 109.9 | 6.51 |
| 6 | Inosine | 269.1 | 137 | 4.02 |
| 7 | N-phenylacetylglycine | 194.2 | 91.1 | 13.01 |
| 8 | Cholic acid | 409.3 | 373.3 | 16.93 |
| 9 | Taurine | 126 | 126 | 8.6 |
| 10 | Ursodeoxycholic acid(UDCA) | 391.28 | 391.28 | 16.24 |
| 11 | Glycocholic acid | 464.3 | 464.3 | 17.27 |
| 12 | Glycochenodeoxycholic acid(GCDCA) | 450.4 | 450.4 | 17.83 |
| 13 | LCA(lithocholic acid) | 375.3 | 375.3 | 13.23 |
| 14 | Taurocholic acid | 514.3 | 514.3 | 17.86 |
| 15 | Taurochenodexycholic acid | 498.3 | 498.3 | 17.36 |
| 16 | LPC(20:4) | 588.5 | 588.5 | 18.64 |
| 17 | LPC (16:1) | 494.3 | 494.3 | 18.55 |
| 18 | LPC(18:1) | 522.4 | 522.4 | 18.99 |
| 19 | LPE(18:0) | 482.3 | 482.3 | 18.86 |
| 20 | LPC(16:0) | 496.3 | 496.3 | 18.86 |
| 21 | PC(16:0/0:0) | 496.3 | 496.3 | 18.78 |
| 22 | LPC(18:0) | 524.4 | 524.4 | 17.8 |
| 23 | PE(36:4) | 740.6 | 740.5 | 14.64 |
| 24 | PE(38:4) | 768.6 | 768.6 | 16.54 |
| 25 | LPC(20:1) | 550.4 | 550.4 | 18.86 |
| 26 | LPC(22:6) | 568.3 | 568.3 | 18.6 |
| 27 | PC(36:3) | 784.6 | 784.6 | 14.73 |
| 28 | 2-hydroxyglutarate(2-HG) | 147.1 | 129.2 | 1.34 |
| 29 | Palmitic acid | 255 | 255 | 13.54 |
| 30 | 3-hydroxybutyric | 103 | 59 | 2.37 |
| 31 | Ethlymalonic acid | 131 | 86.9 | 6.47 |
| 32 | Acetylcarnitine | 204.1 | 204.1 | 1.28 |
| 33 | Aminohippuric acid | 195.1 | 195.1 | 7.83 |
| 34 | Palmitoyl-L-carnitine | 400.3 | 400.3 | 17.74 |
| 35 | Choline | 104.2 | 60.3 | 0.75 |
| 36 | Glutamic | 147.1 | 130.1 | 1.63 |
| 37 | Glutathione, Oxidized(GSSG) | 613.2 | 355.3 | 2.93 |
| 38 | Glutathione(GSH) | 433.1 | 304.1 | 11.89 |
| 39 | L-Leucine | 132.1 | 86.1 | 2.48 |
| 40 | L-Valine | 118.1 | 118.1 | 1.19 |
| 41 | Hippuric acid | 180.1 | 180.1 | 12.32 |
| 42 | Phenylalanine | 166.1 | 120.1 | 4.57 |
| 43 | Creatine | 132 | 90 | 0.87 |
| 44 | Spermidine | 146.1 | 146.1 | 7.38 |
| 45 | Creatinine | 114.5 | 44 | 0.82 |
| 46 | Proline | 116.1 | 116.1 | 0.89 |
| 47 | Alanine | 90.1 | 44.2 | 0.75 |
| 48 | L-Tryptophan | 205.2 | 188.1 | 10.69 |
| 49 | L-Kynurenine | 209.1 | 192.1 | 4.4 |
| 50 | Xanthurenic acid | 204 | 204 | 14.35 |
| 51 | 5-hydroxytryptophan(5-HTP) | 221.1 | 204.3 | 3.63 |
| 52 | 5-hydroxytryptamine(5-HT) | 177.1 | 160.1 | 3.27 |
| 53 | 3-chloro-Ltyrosine(Cl-Tyr) | 216 | 170 | 4.31 |
| 54 | DL-o-tyrosine(o-Tyr) | 182.1 | 136.1 | 1.99 |
| 55 | Carnitine | 162.1 | 162.1 | 0.78 |
| 56 | S-(adenosyl)-L-homocysteine(SAH) | 385.1 | 136 | 2.85 |
| 57 | 4-(2-aminophenyl)-2,4-dioxobutanoic acid | 208.1 | 162.1 | 14.04 |
| 58 | 4,6-dihydroxyquinoline | 162.1 | 116 | 2.13 |
| 59 | p-Cresol Glucuronide | 283.08 | 107 | 13.74 |
| 60 | Trigonelline | 138.1 | 138.1 | 1.39 |
| 61 | pyridoxic acid | 182.1 | 182.1 | 0.7 |
| 62 | Pantothenic acid | 218.1 | 218.1 | 9.45 |
| 63 | Citric acid | 191 | 111.2 | 1.4 |
| 64 | Oxaloacetate | 130.8 | 86.9 | 5.91 |
| 65 | Succinic acid | 117 | 73 | 2.11 |
| 66 | a-Ketoglutaric acid | 145.1 | 101 | 1.18 |
| 67 | Malic acid | 132.9 | 115 | 1.04 |
| 68 | Fumaric acid | 115 | 70.9 | 1.6 |
| 69 | Hydroxybutanedioic acid | 133 | 115 | 1.02 |
| 70 | Glucosamine-6-phosphate | 260 | 126 | 1.87 |
| 71 | Glucose | 179.1 | 89.1 | 1.26 |
| 72 | Lactate | 89 | 89 | 1.24 |

Blank plasma based quality control (QC) standards were prepared from rat plasma which had been stripped of endogenous materials by adding 6 g/100 mL charcoal activated powder (Actived CharcoalNorit, Sigma-Aldrich, St. Louis, Mo., USA). This suspension was stirred at room temperature for 2 h and centrifuged 20 min at 135.00 rpm at 4° C. And the supernatant was filtered using Millipore Express PES Membrane (Merck Millipore Ltd., Germany). The obtained “stripped” plasma was confirmed by LC-MS/MS to be free of biomarker. The QC samples with three different concentrations were generated by adding L-tryptophan (Try), L-kynurenine (Kyn), GSSG, N-phenylacetylglycine (N-Phe), 5-HTP, L-leucine (Leu), 5-HT, cholic acid (CA) and GSH standard solutions to “stripped” plasma, and then processed in the same way as in vivo samples. The standard solutions of Try, Kyn, GSSG, N-Phe, 5-HTP, 5-HT, Leu, CA stock solutions were prepared at 1 mg/ml in 100% methanol, except GSH were prepared at 0.5 mg/ml in 10 mM NEM PBS buffer. The standard curves with eight different concentrations were generated in the same way as QC samples, and analyzed by 1/X weighted least squares linear regression. A series of QC samples as well as standard curves were running every 50 animal samples.

The normalized data were exported and further processed by PCA and PLS-DA using a multivariate analysis package SIMCA-P software (Umea, Sweden). Model quality was evaluated based on the relevant values of R² and Q². Potential markers of interest were extracted from the values of variable importance in the projections (VIP>1), which were constructed from PLS-DA analysis. P values were obtained from Student's t-test (P<0.05). Hierarchical clustering analysis (HCA) was performed using bioinformatics software Multi Experiment Viewer (The Institute of Genomic Research, MA, USA) for visualization and organization of metabolite profiles.

Quantitative Real Time (RT)-PCR Analysis

Total RNA was isolated from the frozen liver of the AIA model using TRIzol® Reagent (Life technology, USA). mRNA was purified by binding to poly(dT) magnetic beads using Dynabeads® mRNA DIRECT™ Kit (Life technology, Norway) and reverse transcribed using SuperScript® III reverse transcriptase (Invitrogen, California, USA) as described by the manufacturer. Quantitative real-time PCR was performed using SYBR Green (Sigma-Aldrich, Louis, USA) on the ViiA™ 7 Real-Time PCR System (Applied Biosystems, FosterCity, USA). Quantitative RT-PCR analysis was performed using SYBR Green reagents and ViiA™ 7 Real-Time PCR System. Gene expression levels were calculated from cycle threshold (Ct) values and normalized with control gene GAPDH. The primer sequences have been listed in Table 2.

TABLE 2
Primers used for the RT-PCR

| | | SEQ. | |
|---|---|---|---|
| | | ID. | |
| Protein | Gene | NO: | Primer Sequence |
| HMGCR | Hmgcr | 1 | Forward: 5′-TAGAGATCGGAACCGTGGGT-3′ |
| | | 2 | Reverse: 5′-GCCCCTTGAACACCTAGCAT-3′ |
| SREBP1 | Srebf1 | 3 | Forward: 5′-GCGTGGTTTCCAACATGACC-3′ |
| | | 4 | Reverse: 5′-TCCTTTGCCACTGGAACCTG-3′ |
| PPAR alpha | Ppara | 5 | Forward: 5′-AAGTTTGCCAGTTGGGGTCA-3′ |
| | | 6 | Reverse: 5′-GAAGCATCCGTCTTCACCGA-3′ |
| LXR alpha | Nr1h3 | 7 | Forward: 5′-TTTCTCCTGACTCTGCAACGG-3′ |
| | | 8 | Reverse: 5′-GAGGCCTTGTCCCCACATAC-3′ |
| FXR | Nr1h4 | 9 | Forward: 5′-TTCGAAAGAGCGGCATCTCC-3′ |
| | | 10 | Reverse: 5′-TAGGACATCGAGCAGAGGCT-3′ |
| SR-B1 | Scarb1 | 11 | Forward: 5′-TTCGAACAGAGCGGGATGAT-3′ |
| | | 12 | Reverse: 5′-CCTTATCCTGCGAGCCCTTT-3′ |
| LPL | Lpl | 13 | Forward: 5′-AAGAAGTCGGGCTGACACTGG-3′ |
| | | 14 | Reverse: 5′-GAGGACATGCTATCGGCCATT-3′ |
| CYP7A1 | Cyp7a1 | 15 | Forward: 5′-CTTCTGCGAAGGCATTTGGAC-3′ |
| | | 16 | Reverse: 5′-GGCATACATCCCTTCCGTGA-3′ |
| LDLR | Ldlr | 17 | Forward: 5′-AGACCCAGAGCCATCGTAGT-3′ |
| | | 18 | Reverse: 5′-GGCCACTGGGAAGATCTAGTG-3′ |
| PPAR gamma | Pparg | 19 | Forward: 5′-CTGGCCTCCCTGATGAATAA-3′ |
| | | 20 | Reverse: 5′-GGCGGTCTCCACTGAGAATA-3′ |
| G6PD | G6pd | 21 | Forward: 5′-GAGGAGTTCTTTGCCCGTAAC-3′ |
| | | 22 | Reverse: 5′-ATCTCTTTGCCCAGGTAGTGGT-3′ |
| ACS | Acsl1 | 23 | Forward: 5′-CAGGTGTCAAATGATGGCCC-3′ |
| | | 24 | Reverse: 5′-AGTAAGTGAAGCACCCCTGC-3′ |
| aP2 | Fabp4 | 25 | Forward: 5′-TCGTCATCCGGTCAGAGAGT-3′ |
| | | 26 | Reverse: 5′-ACACATTCCACCACCAGCTT-3′ |
| CPT-1α | Cpt1α | 27 | Forward: 5′-GGAGGTTGTCTACGAGCCAG-3′ |
| | | 28 | Reverse: 5′-CAAAGCGGTGTGAGTCTGTC-3′ |
| CD36/FAT | Cd36/Fat | 29 | Forward: 5′-CTTGGATGTGGAACCCATAACT-3′ |
| | | 30 | Reverse: 5′-CGATGGTCCCAGTCTCATTTAG-3′ |
| CYP2E1 | Cyp2e1 | 31 | Forward: 5′-ACCCTCATCTCTGACCATACT-3′ |
| | | 32 | Reverse: 5′-GTCTTGGGTGTAGGGTGATTT-3′ |
| CYP27A1 | Cyp27a1 | 33 | Forward: 5′-GGTCACATGGTAAGAGGGTATG-3′ |
| | | 34 | Reverse: 5′-GGAGGTAGAAGTAGGTGGATCT-3′ |
| SREBP2 | Srebf2 | 35 | Forward: 5′-TGGAAGGAAGGTAGAGTAGGTGGG-3′ |
| | | 36 | Reverse: 5′-TTTTGTGGACTGCTTGGCTCAGGG-3′ |
| XDH | Xdh | 37 | Forward: 5′-CAAGATTGTCAGCAATGCATCC-3′ |
| | | 38 | Reverse: 5′-ATCACGCCACAGCTTTCCAGAG-3′ |
| XO | Xo | 39 | Forward: 5′-CGCAGAATACTGGATGAGCGAGGT-3′ |
| | | 40 | Reverse: 5′-GCCGGTGGGTTTCTTCTTCTTGAA-3′ |
| HGPRT | Hprt1 | 41 | Forward: 5′-CTCATGGACTGATTATGGACAGGAC-3′ |
| | | 42 | Reverse: 5′-GCAGGTCAGCAAAGAACTTATAGCC-3′ |
| PAH | Pah | 43 | Forward: 5′-GAAACTGGCCACAATTTACTGGT-3′ |
| | | 44 | Reverse: 5′-CTGAAACTCTCCGCCACGTA-3′ |
| GSH-Px | Gpx1 | 45 | Forward: 5′-GCTCACCCGCTCTTTACCTT-3′ |
| | | 46 | Reverse: 5′-GATGTCGATGGTGCGAAAGC-3′ |
| CETP | Cetp | 47 | Forward: 5′-AATAAGGGCGTCGTGGTCAG-3′ |
| | | 48 | Reverse: 5′-AGCCTCAGACTCATTGGAAGC-3′ |
| FATP | Slc27a1 | 49 | Forward: 5′-CCAGAGAAGGATGCGGACTC-3′ |
| | | 50 | Reverse: 5′-GTGTCGTCGTAGCTCTAGCC-3′ |
| ApoC2 | Apoc2 | 51 | Forward: 5′-GTGTTGGGAAACGAGGTCCAG-3′ |
| | | 52 | Reverse: 5′-TGGTCTAGAGTTGGACGCAG-3′ |
| ApoE | Apoe | 53 | Forward: 5′-GTCCCATTGCTGACAGGATGC-3′ |
| | | 54 | Reverse: 5′-CGAGTCGGTTGCGTAGATCC-3′ |
| BSEP | Abcb11 | 55 | Forward: 5′-GCCAGATGAGTGGTGGTCAG-3′ |
| | | 56 | Reverse: 5′-GCATCTTTCCCCATTATGCTCG-3′ |
| GAPDH | Gapdh | 57 | Forward: 5′-AGCTCATTTCCTGGTATGACAA-3′ |
| | | 58 | Reverse: 5′-GGTATTCGAGAGAAGGGAGGG-3′ |

Western Blot Analysis

Liver tissue homogenates of AIA and MCD animal models were lysed in RIPA buffer (50 mM pH 7.4 Tris, 150 mM NaCl, 1% Triton X-100, 1% sodium deoxycholate, 0.1% SDS, sodium orthovanadate, sodium fluoride and EDTA) containing protease inhibitor cocktails (Roche Life Science, USA). Protein concentration was determined using the BCA protein assay kit (BIO-RAD, USA). Equal amounts of total protein were resolved using SDS-PAGE and transferred onto PVDF membranes (Millipore, Darmstadt, Germany). After incubation in a blocking solution containing with 5% (w/v) skim milk (Nestle Carnation, New Zealand) in TBST buffer (10 mM tris-buffered saline and 0.1% Tween20) for 1 h at room temperature and incubated with primary antibodies overnight at 4° C. The membranes were washed three times with 1×TBST solutions and incubated with the appropriate secondary antibodies at room temperature for 45 min, and subsequently visualized with an enhanced chemiluminescence detection kit (SuperSignal™ West Pico Chemiluminescent Substrate, Thermo Scientific, USA). β-actin was used as the loading control for the experimental data analysis.

Results

Silybin Significantly Ameliorates Adjuvant-Induced Arthritis (AIA) in Rats

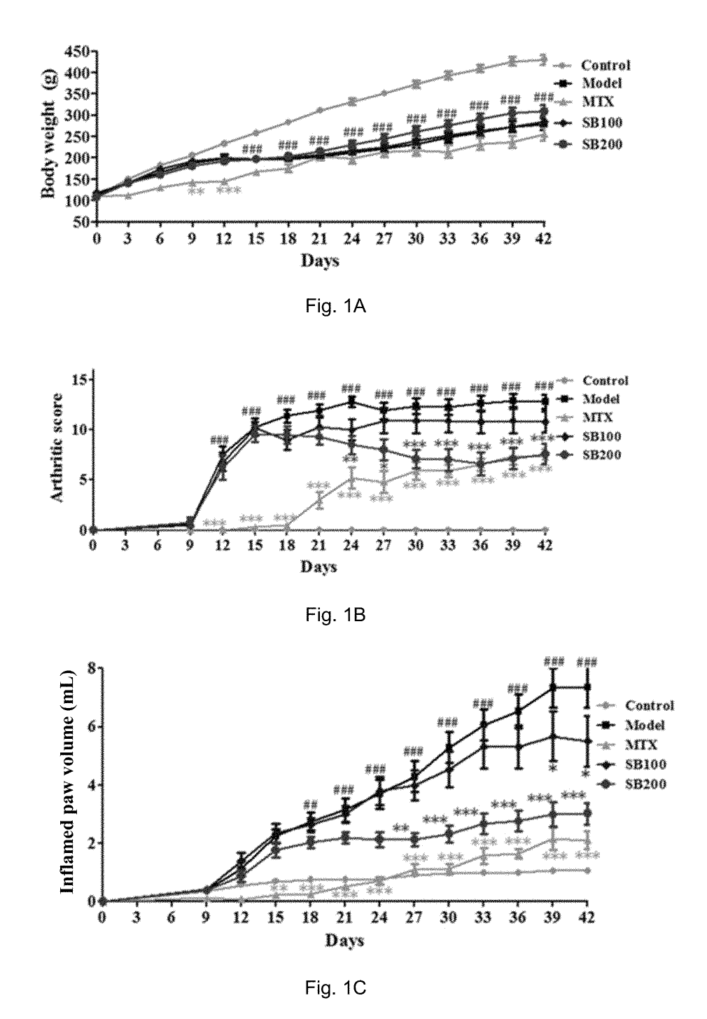

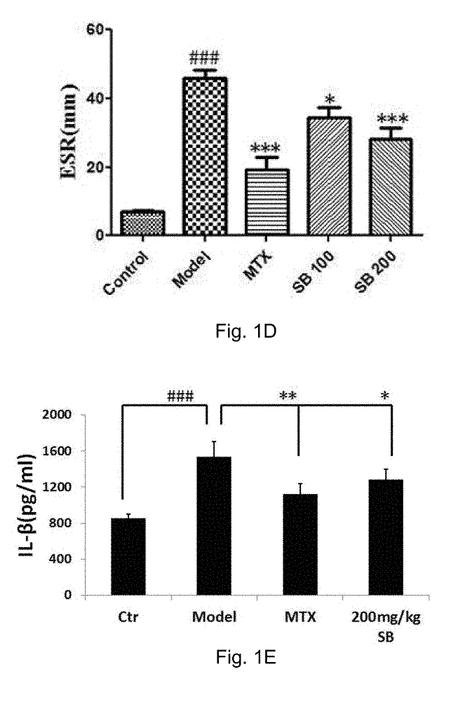

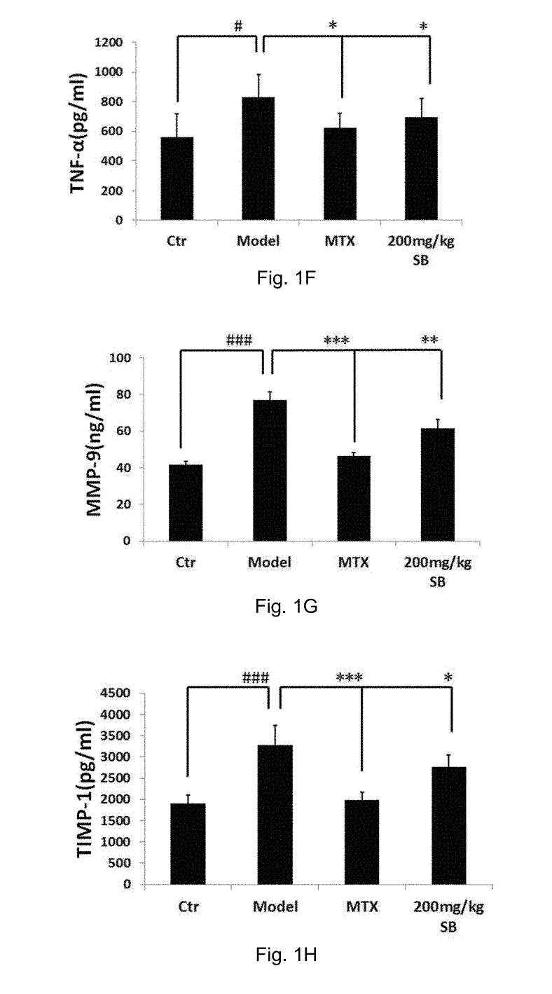

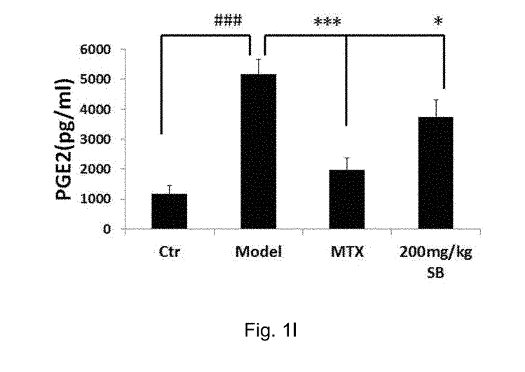

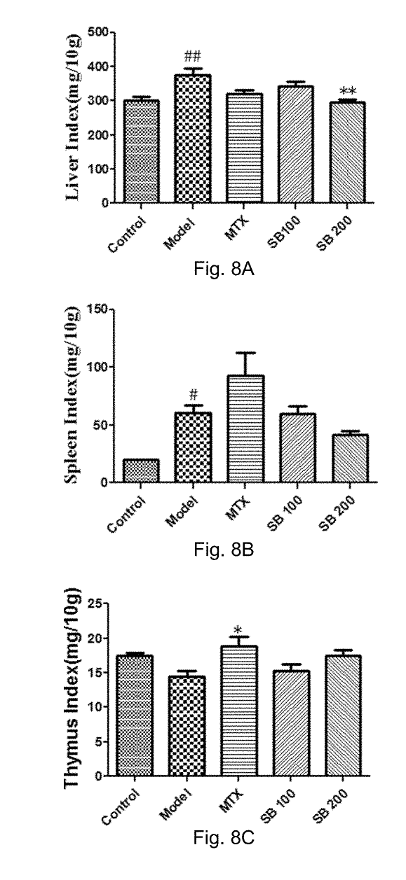

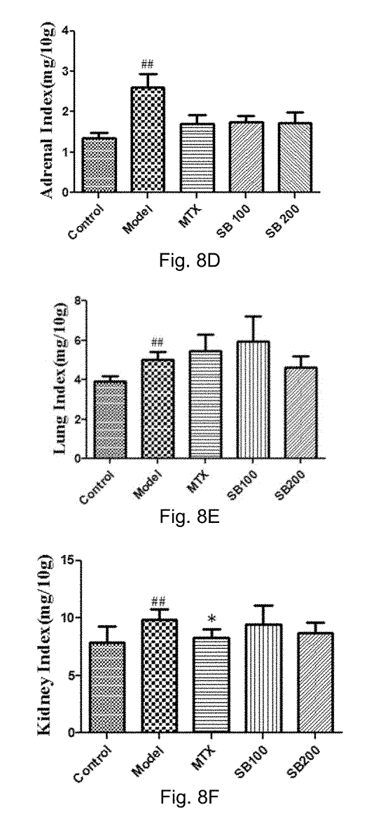

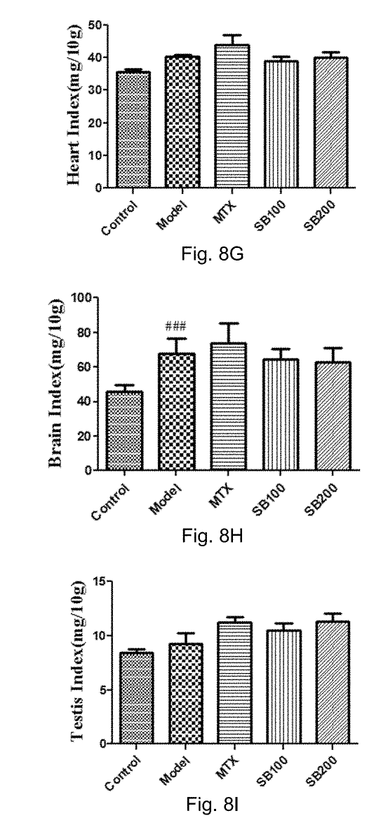

As RA is a systemic disease, body weight loss as a major clinical finding was measured during the period of this experiment. In AIA model rats treated with vehicle, reduced weights were noticed during day-12 to day-42 as compared to normal rats, while the body weight was further reduced by treatment with MTX (FIG. 1A). Silybin (200 mg/kg) significantly (P<0.001) increased the body weight by comparing with that of vehicle-treated AIA rats, suggesting that Silybin does not cause a toxic response. Moreover, Silybin reversed changes in the relative organ weight ratio (such as liver index, spleen index, kidney index, lung index) in AIA rat model as shown in FIG. 8A to 8I, indicating that Silybin may have beneficial protective effects on multiple organs (see also Tables 3 to 11). The total arthritis score and inflamed paw volume were increased significantly on day 12 after induction of arthritis (FIGS. 1B and 1C). MTX treatment alone significantly (p<0.001) ameliorated the changes in the paw volume and arthritis score as compared to the changes in arthritic vehicle-treated AIA rats. Treatment with Silybin at 200 mg/kg showed anti-edematous effects evidenced by significant decrease in arthritis score and paw volume till reaching 7.60 and 3.02 ml, respectively on day 42 (FIGS. 1B and 1C). Furthermore, anti-arthritic, i.e. anti-inflammatory effects of Silybin were manifested as significant increases in all arthritic parameters. As shown in FIG. 1D and in Table 12, compared to the vehicle-treated AIA rats, the group treated with MTX or 200 mg/kg of Silybin showed significantly (P<0.001) reduced ESR values. ELISA assays showed that Silybin caused a significant decrease in serum levels of TNF-α, IL-1β, MMP-9, TIMP-1, and PGE2 which were significantly up-regulated in the vehicle-treated AIA rats, but not IL-33 (FIGS. 1E to 1I).

TABLE 3
Changes in the relative organ weight ratio (Liver Index)

| | | Liver Index |
|---|---|---|
| Group | n | (mg/10 g) ± SD |
| Control | 11 | 300.40 ± 36.09 |
| Model | 11 | 376.20 ± 60.72## |
| MTX | 10 | 320.10 ± 35.41 |
| SB100 | 10 | 341.40 ± 50.42 |
| SB200 | 10 | 296.20 ± 23.28** |

TABLE 4
Changes in the relative organ weight ratio (Spleen Index)

| | | Spleen Index |
|---|---|---|
| Group | n | (mg/10 g) ± SD |
| Control | 11 | 19.91 ± 1.84 |
| Model | 11 | 60.29 ± 22.04# |
| MTX | 10 | 93.15 ± 61.77 |
| SB100 | 10 | 60.03 ± 18.98 |
| SB200 | 10 | 41.92 ± 10.48 |

TABLE 5
Changes in the relative organ weight ratio (Thymus Index)

| | | Thymus Index |
|---|---|---|
| Group | n | (mg/10 g) ± SD |
| Control | 11 | 17.48 ± 1.64 |
| Model | 11 | 14.54 ± 2.34 |
| MTX | 10 | 18.83 ± 4.26* |
| SB100 | 10 | 15.26 ± 3.32 |
| SB200 | 10 | 17.51 ± 2.60 |

TABLE 6
Changes in the relative organ weight ratio (Adrenal Index)

| | | Adrenal Index |
|---|---|---|
| Group | n | (mg/10 g) ± SD |
| Control | 11 | 1.35 ± 0.41 |
| Model | 11 | 2.60 ± 1.11## |
| MTX | 10 | 1.71 ± 0.69 |
| SB100 | 10 | 1.74 ± 0.49 |
| SB200 | 10 | 1.73 ± 0.83 |

TABLE 7
Changes in the relative organ weight ratio (Lung Index)

| | | Lung Index |
|---|---|---|
| Group | n | (mg/10 g) ± SD |
| Control | 11 | 3.93 ± 0.29 |
| Model | 11 | 5.05 ± 0.39 |
| MTX | 10 | 5.47 ± 0.84* |
| SB100 | 10 | 5.95 ± 1.27 |
| SB200 | 10 | 4.62 ± 0.59 |

TABLE 8
Changes in the relative organ weight ratio (Kidney Index)

| | | Kidney Index |
|---|---|---|
| Group | n | (mg/10 g) ± SD |
| Control | 11 | 7.85 ± 1.43 |
| Model | 11 | 9.87 ± 0.89## |
| MTX | 10 | 8.29 ± 0.74 |
| SB100 | 10 | 9.46 ± 1.62 |
| SB200 | 10 | 8.74 ± 0.84 |

TABLE 9
Changes in the relative organ weight ratio (Heart Index)

| | | Heart Index |
|---|---|---|
| Group | n | (mg/10 g) ± SD |
| Control | 11 | 35.59 ± 3.11 |
| Model | 11 | 40.22 ± 2.13 |
| MTX | 10 | 43.98 ± 9.18 |
| SB100 | 10 | 38.97 ± 4.32 |
| SB200 | 10 | 39.98 ± 5.32 |

TABLE 10
Changes in the relative organ weight ratio (Brain Index)

| | | Brain Index |
|---|---|---|
| Group | n | (mg/10 g) ± SD |
| Control | 11 | 45.80 ± 4.19 |
| Model | 11 | 67.98 ± 8.84### |
| MTX | 10 | 73.97 ± 11.26 |
| SB100 | 10 | 64.54 ± 6.05 |
| SB200 | 10 | 62.97 ± 8.37 |

TABLE 11
Changes in the relative organ weight ratio (Testis Index)

| | | Testis Index |
|---|---|---|
| Group | n | (mg/10 g) ± SD |
| Control | 11 | 8.42 ± 1.10 |
| Model | 11 | 9.29 ± 3.33 |
| MTX | 10 | 11.27 ± 1.55 |
| SB100 | 10 | 10.49 ± 2.05 |
| SB200 | 10 | 11.32 ± 2.50 |

TABLE 12
ESR of AIA rats by treatment with Silybin

| | | ESR |
|---|---|---|
| Group | N | (mm) ± SD |
| Control | 11 | 7.00 ± 0.76 |
| Model | 11 | 45.88 ± 7.83### |
| MTX | 10 | 19.38 ± 11.24*** |
| SB 100 | 10 | 34.35 ± 9.25* |
| SB 200 | 10 | 28.09 ± 11.14*** |

^{#,} *p < 0.05, ^{##,} **p < 0.01, ###, ***p < 0.001 versus normal rats or versus the vehicle-treated rats.

Silybin Prevents Cartilage and Bone from Destruction in AIA Rats

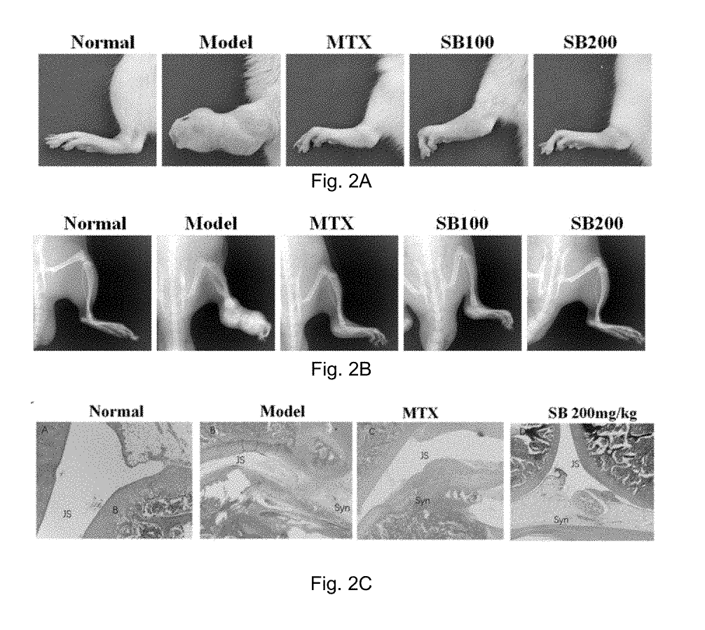

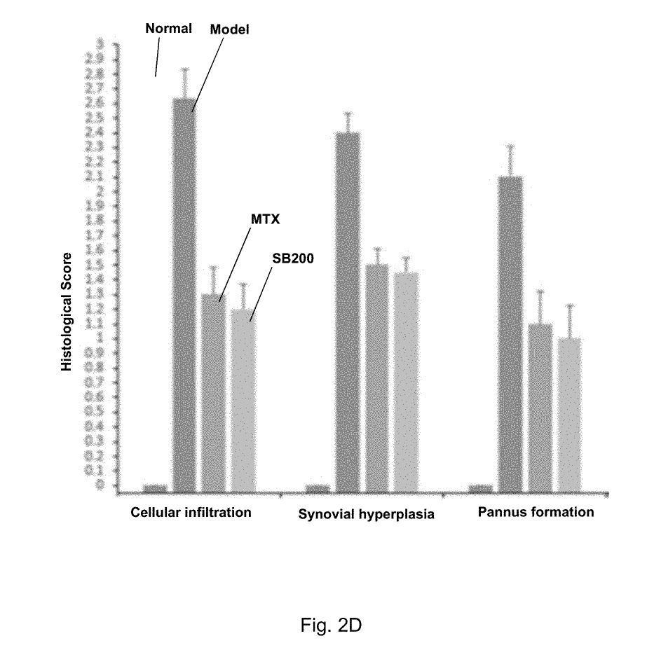

Bone and cartilage destruction, which is a common feature of murine collagen arthritis, were examined by radiological histopathological examinations at the end of the experiments. Representative radiographs and photographs of the hind paw from normal, vehicle-treated AIA, MTX-treated and Silybin-treated rats are shown in FIGS. 2A and 2B. The radiographic results revealed severe soft tissue swelling around the joint and bone erosion in the vehicle-treated AIA rats as compared to the joints of normal rats (FIG. 2B). Administration of Silybin (100 mg/kg or 200 mg/kg) as well as MTX ameliorated the cartilage and joint destruction of the arthritic joints.

Histological analysis confirmed the anti-arthritic effects and radiological findings. Representative histological sections of the ankle joints from normal (grade 0), vehicle-treated AIA (grade 2/3), MTX-treated (grade 0) and Silybin-treated (grade 0) rats are shown in FIG. 2C, respectively In contrast to normal rats, the sections from vehicle-treated AIA rats revealed a markedly thickened and expanded synovium with intense infiltration of inflammatory cells, hyperplasia, prominent cartilage, and pannus formation. Treatment with Silybin or MTX diminished the infiltration of inflammatory cells, synovial hyperplasia, with little cartilage (FIG. 2C). These histopathological findings matched the results of the histological scoring (FIG. 2D).

Silybin Protects the Liver Functions in AIA Rats

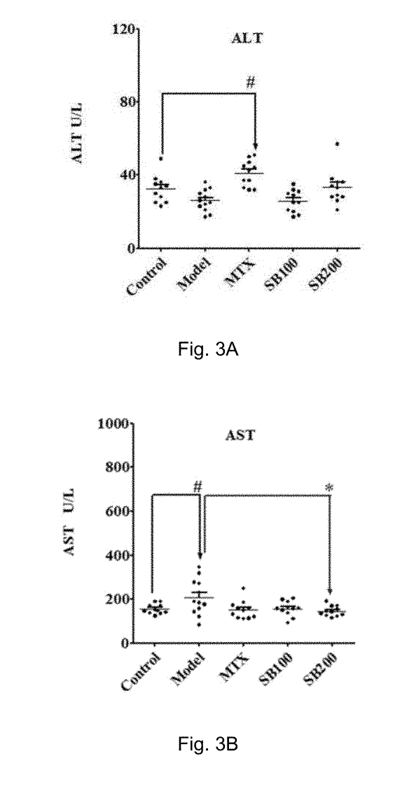

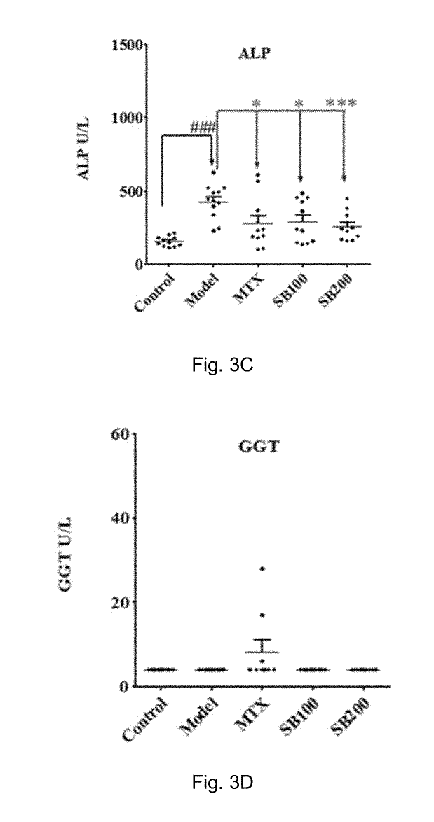

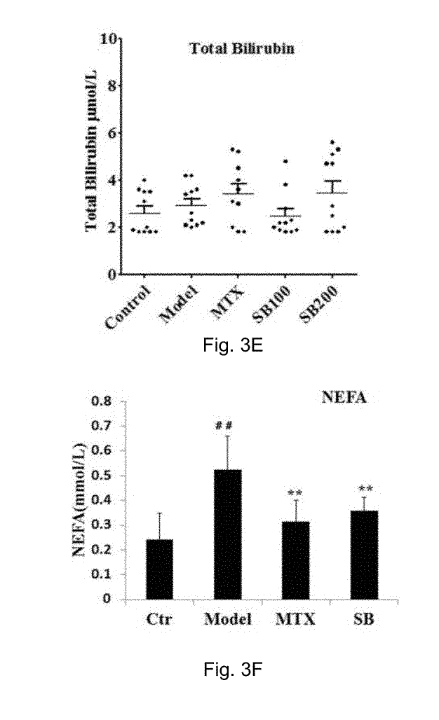

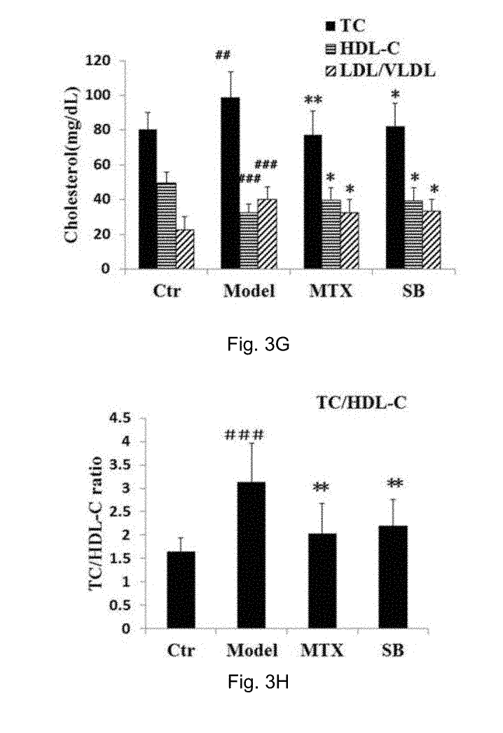

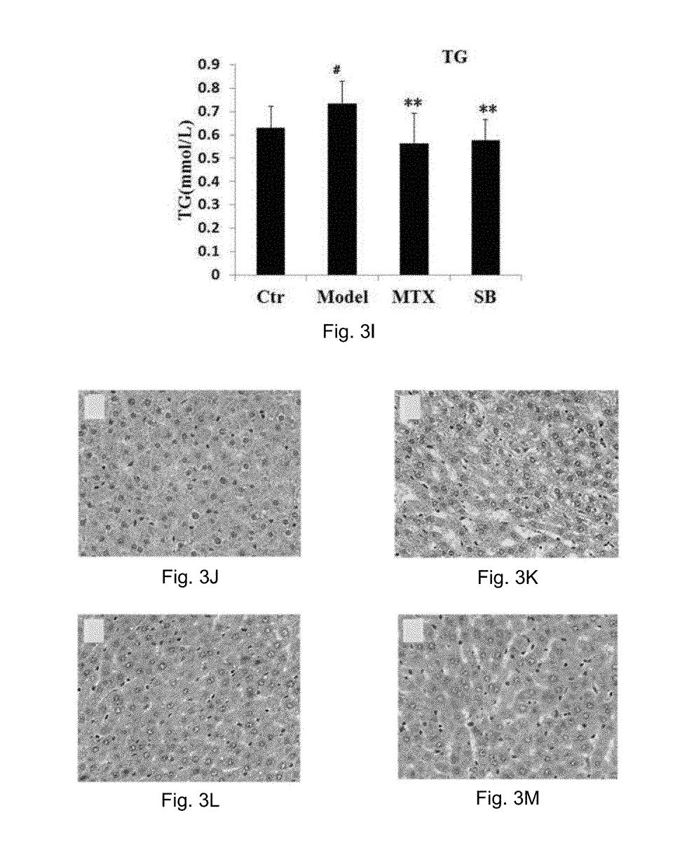

Vehicle-treated AIA model rats exhibited significantly increased ALP levels (P<0.001) and AST level (P<0.05) compared to normal rats (FIGS. 3B and 3C), which is consistent with the phenomenon observed in clinic (Aida, S., Ann Rheum Dis, 1993, 52(7): p. 511-6). Methotrexate treatment alone further induced significant increase in both ALT and GGT serum levels as shown in FIG. 3A to 3D indicating that MTX has adverse impairment to the liver function of the animal that is in line with the side effects of MTX in treating RA patients in clinic. However, treatment of arthritis AIA rats with Silybin in 200 mg/kg markedly reduced AST (P<0.05) and ALP (P<0.001) levels, which was more obvious than that of treatment with 100 mg/kg (P<0.05) (FIG. 3A to 3E).

Liver toxicity was further evaluated by histopathological assessment of liver tissue in different groups. Normal rats showed normal histological structure of the central vein with normal surrounding hepatocytes. Arthritic AIA vehicle-treated rats showed mild degenerative changes in hepatocytes, while congested portal tract and mild fatty changes with lipid drop lets were observed in liver sections taken from MTX treated rats (FIG. 3J to 3M). Concurrent treatment of Silybin preserved the normal architecture of hepatocytes with light congestion of central vein.

Major Altered Metabolites Pathways Revealed by Metabolomic Study in AIA Rats

An atherogenic lipid profile characterized by high total cholesterol and triglyceride levels, and low HDL-C levels was more prevalent in blood of early RA patients who later developed activated RA (van Halm, V. P., et al., Ann Rheum Dis, 2007, 66(2): p. 184-8). The serum levels of TC, TG, and LDL-C/vLDL-C were increased, but level of HDL-C was significantly decreased in the arthritis AIA model group (32.57±4.87) in comparison with the normal group (49.72±6.30) (FIG. 3H), which was consistent with the phenomenon seen in early RA disease. The TC to HCL-C ratio was higher in AIA group than normal (P<0.001) and treatment groups (P<0.01). The difference of free fatty acids (FA) was statistically significant between AIA and normal group. Interestingly, treatment with MTX and Silybin in dosage of 200 mg/kg successfully improved the dyslipidemia as well as the arthritis as shown in FIG. 3F to 3I.

Metabolomics not only helps to reveal the scientific basis of treatment of Silybin, but is also beneficial to understanding the pathogenesis of RA disease. Thus, plasma samples of rat AIA model were analyzed by LC-MS/MS for evaluation of the altered metabolic profiles in the absence or presence the treatment of Silybin. The metabolomics method validation study was carried out with a series of QC specimens. The results showed that the relative standard deviation (RSD) of the spiked QC samples at low, medium and high concentrations were <10% (n=6) and the mean accuracies of low, medium and high QC samples were from 87.22-110.21% as shown in Table 13. These results demonstrate that the system had excellent stability and repeatability during the analysis procedure. Total 72 biomarker peaks were identified, which were listed in Table 1 above

TABLE 13
Precision and accuracy values for determination of Try, Kyn,
GSH, GSSG, 5-HT, 5-HTP, N-Phe and CA in QC samples by spiked
standard solution with blank plasma (method 1, n = 6)
or in QC samples by pooled animal plasma (method 2, n = 8)

| | Method 1 | | | | |
|---|---|---|---|---|---|
| | Concen- | | | Method 2 | |
| | tration | Accuracy | Precision | | Precision |
| metabolite | (ng/mL) | (%) | (%) | Group | (%) |
| GSH | 750 | 98.26 | 5.69 | Low ^{a} | 3.68 |
| | 1000 | 104.5 | 4.31 | Medium ^{b} | 4.13 |
| | 5000 | 88.25 | 2.98 | High ^{c} | 3.87 |
| GSSG | 1500 | 88.69 | 3.68 | Low | 5.01 |
| | 2000 | 89.21 | 5.39 | Medium | 4.36 |
| | 10000 | 87.23 | 5.31 | High | 6.25 |
| L-Leucine | 750 | 104.51 | 3.45 | Low | 5.92 |
| | 1000 | 102.25 | 8.92 | Medium | 11.21 |
| | 5000 | 98.54 | 5.42 | High | 9.54 |
| L-Kynurenine | 37.5 | 89.63 | 6.78 | Low | 8.12 |
| | 50 | 88.24 | 5.98 | Medium | 6.19 |
| | 250 | 104.23 | 5.12 | High | 8.87 |
| L-Tryptophan | 4500 | 108.24 | 11.3 | Low | 6.59 |
| | 6000 | 98.32 | 6.57 | Medium | 5.57 |
| | 30000 | 99.11 | 5.12 | High | 4.23 |
| 5-HTP | 1.5 | 101.28 | 9.71 | Low | 2.98 |
| | 2 | 106.87 | 8.95 | Medium | 5.69 |
| | 10 | 88.02 | 5.12 | High | 12.01 |
| Cholic acid | 300 | 110.21 | 6.12 | Low | 10.58 |
| | 400 | 109.54 | 5.31 | Medium | 9.86 |
| | 2000 | 89.21 | 4.26 | High | 11.21 |
| 5-HT | 6 | 96.12 | 6.12 | Low | 9.14 |
| | 8 | 107.37 | 4.32 | Medium | 5.12 |
| | 40 | 110.06 | 3.98 | High | 4.36 |
| N-phenyl- | 1875 | 87.22 | 8.65 | Low | 10.47 |
| acetyl- | 2500 | 91.06 | 4.25 | Medium | 5.23 |
| glycin | 12500 | 97.38 | 3.98 | High | 5.63 |

^{a} Normal sample;
^{b} Normal added with Model sample in 1:1 ratio;
^{c} Model sample.

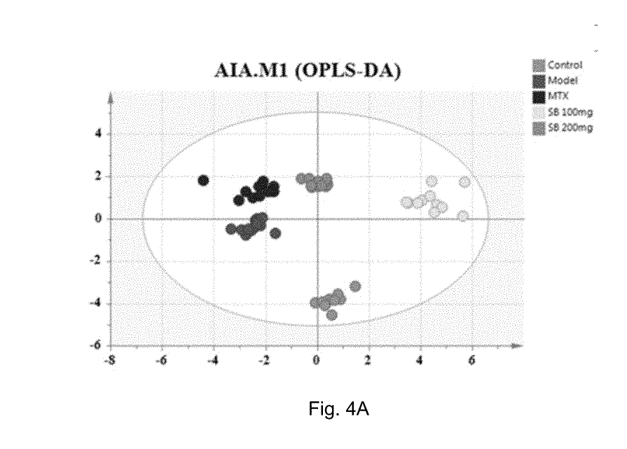

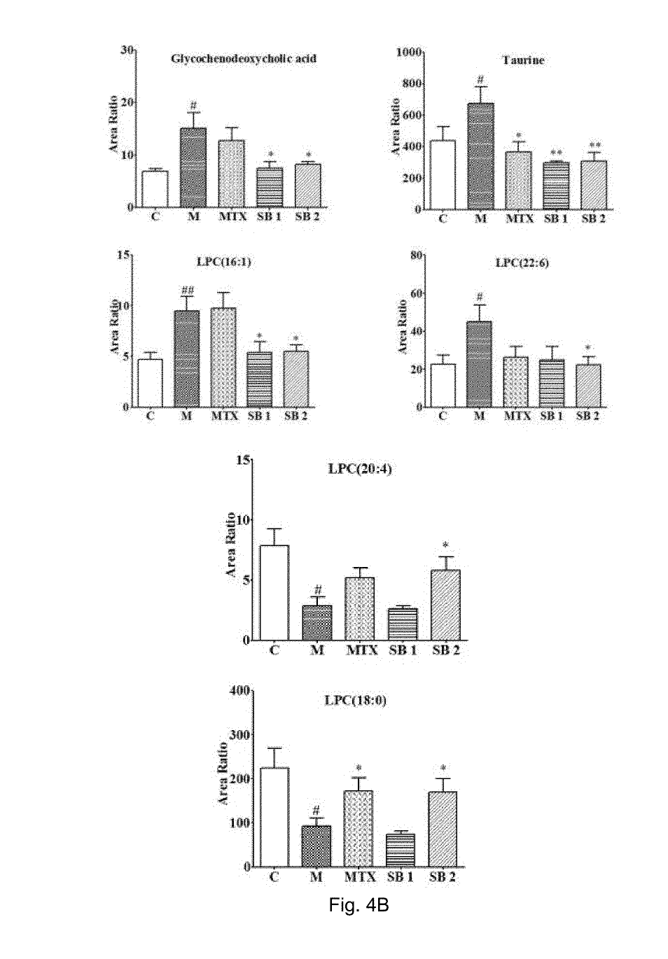

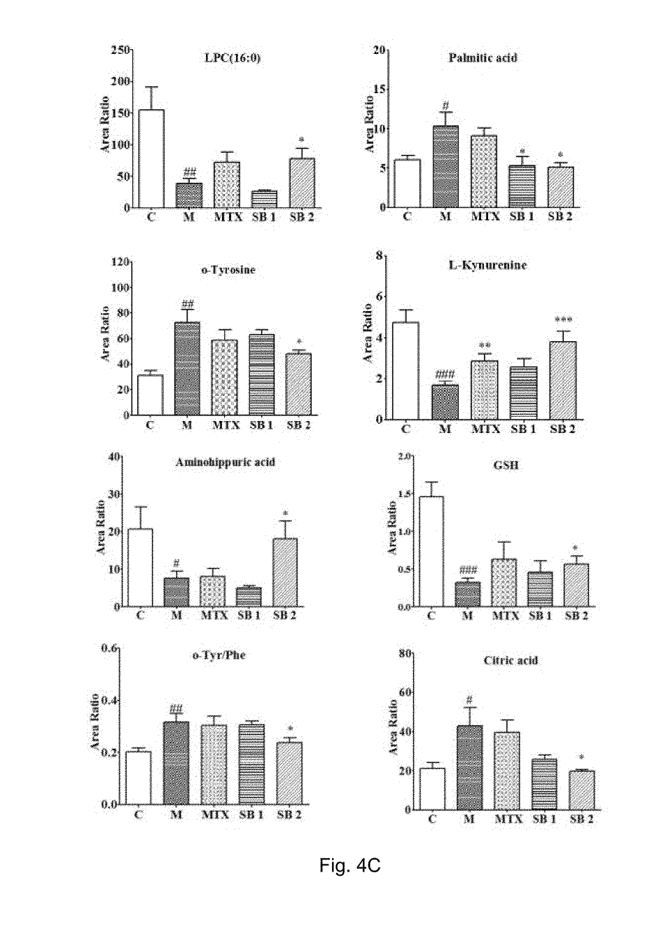

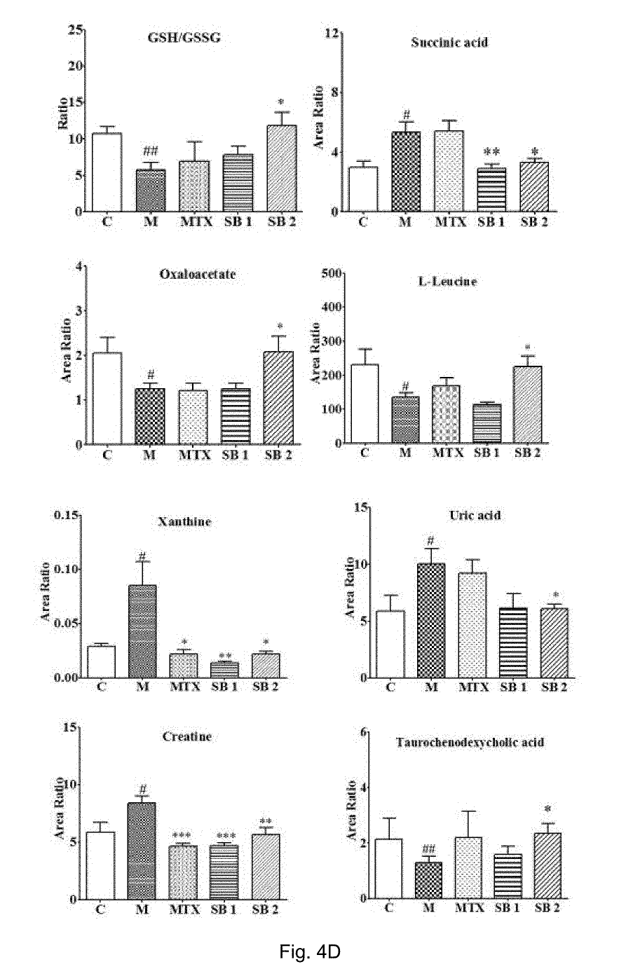

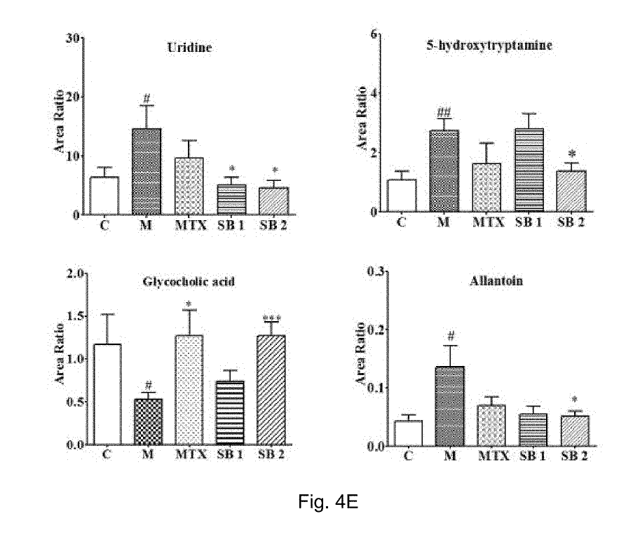

Since principle component analysis (PCA) showed slight discrimination between all groups, OPLS-DA (R²X=0.953, Q²=0.865) was employed for all samples and successfully separated the normal, model and treatment group as shown in FIG. 4A. In the OPLS-DA model, variable importance in the projection (VIP) value was applied to find the potential biomarkers which made the greatest contribution to group separation. And the ions with VIP value above 1.0 and/or p value (t-test) below 0.05 were considered as potential biomarkers. Using hierarchical clustering on the profile of these 72 metabolites revealed a clear separation of AIA rats from normal one as well as treatment groups. By comparing with control rats, 25 endogenous metabolites in plasma were considered to be potential biomarkers that correlated with pathogenesis of arthritis in the AIA model as well as the therapeutic effects of Silybin. The results suggest that the development of arthritis in AIA model involves serious disorders of the metabolism of fatty acids (FAs), phospholipid, amino acid and tricarboxylic acid cycle (TCA cycle). And the analysis of the relative concentration of metabolites revealed that glycochenodeoxycholic acid (GCDCA), taurine, LPC (16:1), LPC (22:6), palmitic acid, xanthine, uric acid, uridine, o-tyrosine, citric acid, succinic acid were elevated in arthritis AIA model, while LPC (20:4), LPC (18:0), LPC (16:0), N-phenylacetylglycine, aminohippuric acid, GSH, L-kynurenine, oxaloacetate were decreased as shown in FIG. 4B to 4E and Table 14. Treatment with Silybin and MTX could effectively repair the metabolic network, thus alleviating the adverse effects of differential metabolites on immune response and inflammation, ameliorating the dysfunction of the immune system and the multi-organ inflammatory response during the development of RA in AIA model, thereby inhibiting the progression of RA.

TABLE 14
Classification of metabolites identified according to metabolic
pathway and comparison of relative levels of metabolites.

| | | | rat AIA model | | mice NASH model | |
|---|---|---|---|---|---|---|
| no | Metabolite | Physiological metabolism | VIP | P-Value* | VIP | P-Value |
| 1 | Glycochenodeoxycholic | Bile acid metabolism | 1.31 | <0.05 ↑ | 1.05 | <0.05 ↑ |
| | acid (GCDCA) | | | | | |
| 2 | Taurine | Bile acid metabolism | 1.09 | <0.05 ↑ | 1.02 | — |
| 3 | LPC(20:4) | Glycerophospholipid | 1.06 | <0.05 ↓ | 1.02 | — |
| | | metabolites | | | | |
| 4 | LPC(16:1) | Glycerophospholipid | 1.11 | <0.01 ↑ | 1.00 | <0.01 ↑ |
| | | metabolites | | | | |
| 5 | LPC(22:6) | Glycerophospholipid | 1.06 | <0.05 ↑ | 1.11 | <0.001 ↑ |
| | | metabolites | | | | |
| 6 | LPC(18:0) | Glycerophospholipid | 1.02 | <0.05 ↓ | 1.00 | <0.001 ↓ |
| | | metabolites | | | | |
| 7 | LPC(16:0) | Glycerophospholipid | 1.00 | <0.01 ↓ | 1.18 | — |
| | | metabolites | | | | |
| 8 | Palmitic acid | Fatty acid metabolism | 1.26 | <0.05 ↑ | 1.20 | — |
| 9 | N-phenylacetylglycine | Fatty acid metabolism | 1.16 | — | 1.10 | <0.001 ↑ |
| 10 | Aminohippuric acid | Fatty acid metabolism | 1.10 | <0.05 ↓ | 1.20 | — |
| 11 | GSSG | Glutathione metabolism | 1.03 | — | 1.02 | <0.05 ↑ |
| 12 | GSH | Glutathione metabolism | 1.30 | <0.001 ↓ | 1.11 | <0.001 ↓ |
| 13 | GSH/GSSG | Glutathione metabolism | 1.00 | <0.01 ↑ | 1.04 | <0.001 ↑ |
| 14 | o-Tyrosine | Amino acid metabolism | 1.13 | <0.01 ↑ | 1.00 | — |
| 15 | L-Kynurenine | Amino acid metabolism | 1.11 | <0.001 ↓ | 1.03 | <0.01 ↓ |
| 16 | o-Tyrosine/phenylalanine | Amino acid metabolism | 1.14 | <0.01 ↑ | 1.02 | <0.001 ↑ |
| 17 | Citric acid | Citric acid cycle | 1.05 | <0.05 ↑ | 0.97 | — |
| 18 | Oxaloacetate | Citric acid cycle | 1.19 | <0.05 ↓ | 1.05 | <0.01 ↓ |
| 19 | Succinic acid | Citric acid cycle | 1.23 | <0.05 ↑ | 0.86 | — |
| 20 | Xanthine | Purine nucleotide | 1.09 | <0.05 ↑ | 0.97 | — |
| | | metabolism | | | | |
| 21 | Uric acid | Purine nucleotide | 1.15 | <0.05 ↑ | 1.01 | <0.001 ↑ |
| | | metabolism | | | | |
| 22 | Uridine | Purine nucleotide | 1.10 | <0.05 ↑ | 1.04 | <0.001 ↑ |
| | | metabolism | | | | |

*P-Values were determined using the Student's t test, * P < 0.05, ** P < 0.01 and ***P < 0.01 VS model or Control.
(n = 10-11 for AIA: N = 6-8 for NASH)

Mechanism Study for Understanding the Disorder of Lipid Metabolism in AIA Rats

Based on the metabolomic results, lipid metabolism is one of the major pathways related with the pathogenesis of arthritis in the AIA model as well as the treatment. To investigate the lipid paradox phenomenon and mechanism by which Silybin reduced hepatic lipid accumulation, expression levels of genes involved in lipid metabolism have been analyzed. Based on the information of potential biomarkers, 22 enzymes closely related with the lipid metabolism were examined in liver tissues from AIA models after Silybin treatment. Among them 17 differentially expressed proteins among normal rat, vehicle-treated AIA rat and Silybin treated group were verified by real-time PCR, while 12 of the 17 enzymes were further verified by Western blotting.

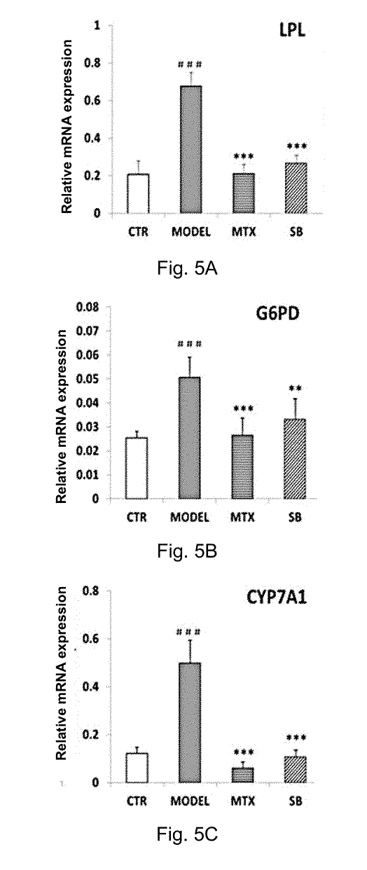

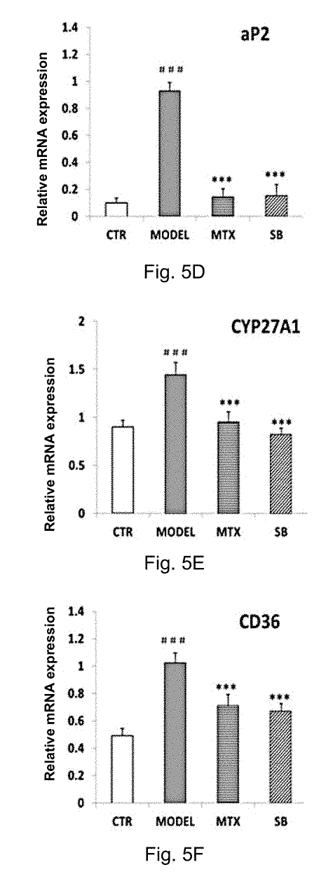

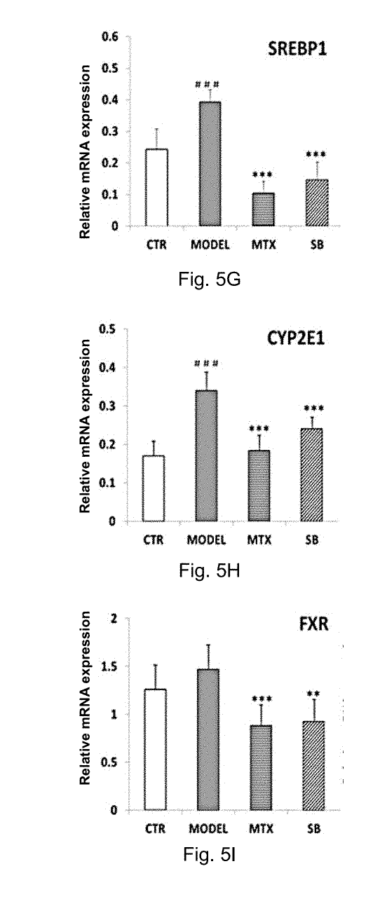

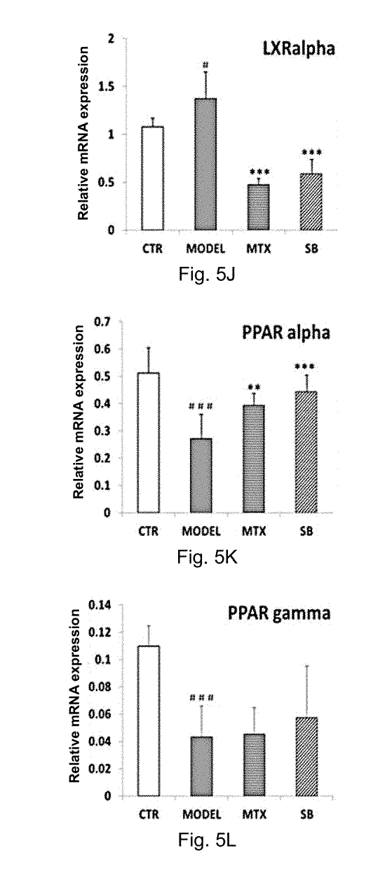

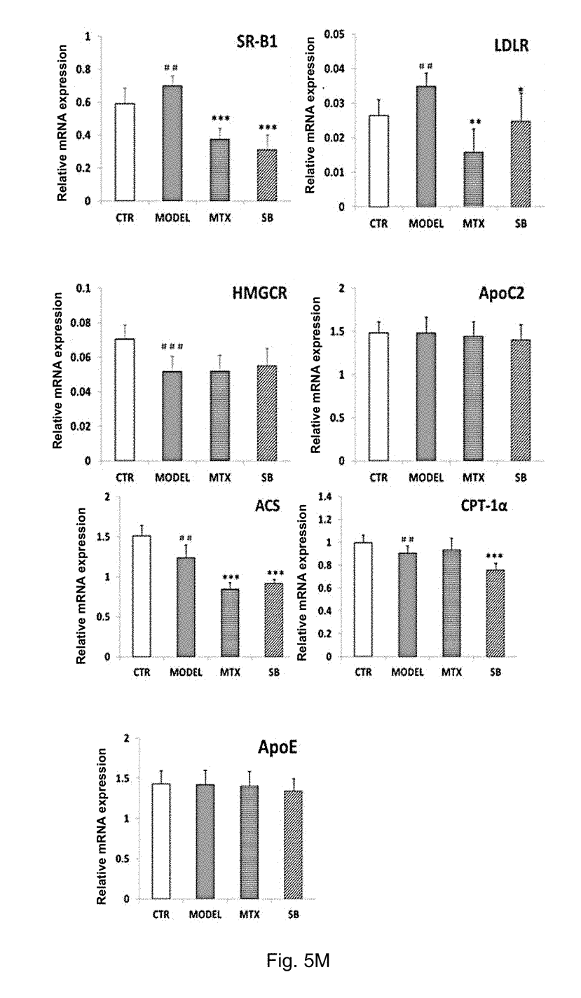

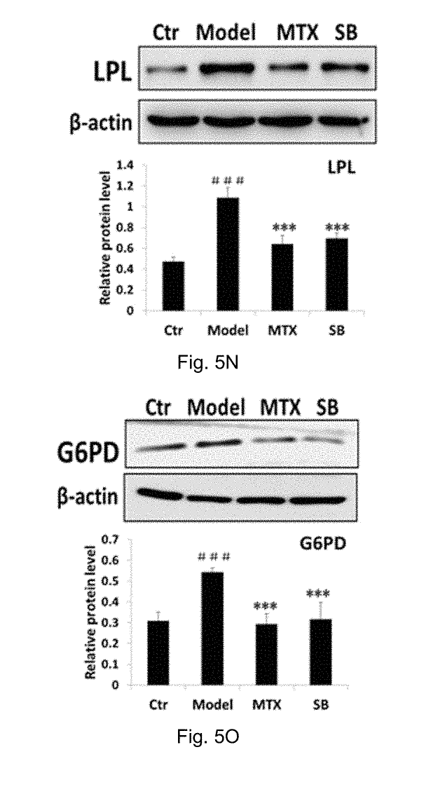

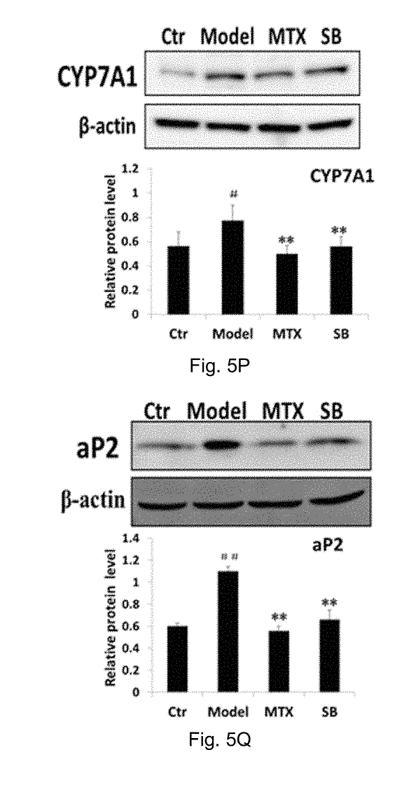

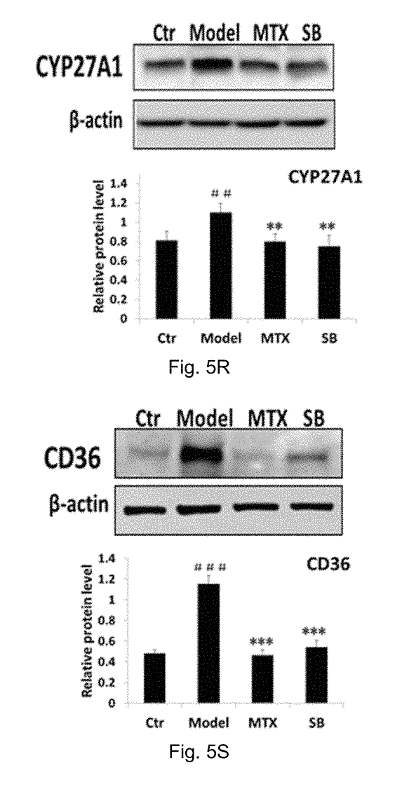

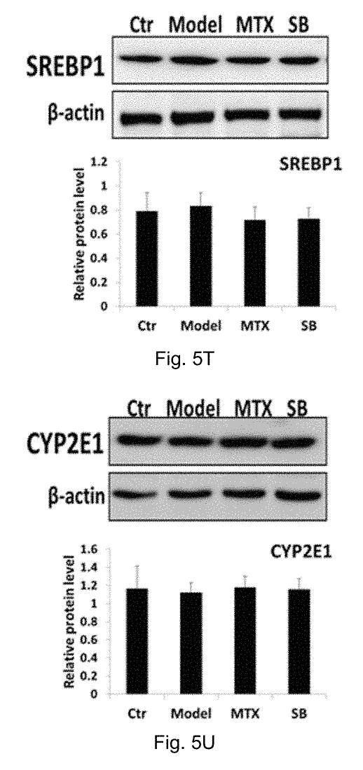

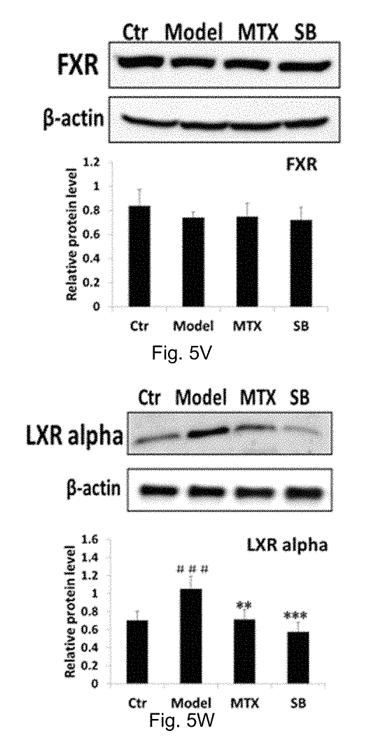

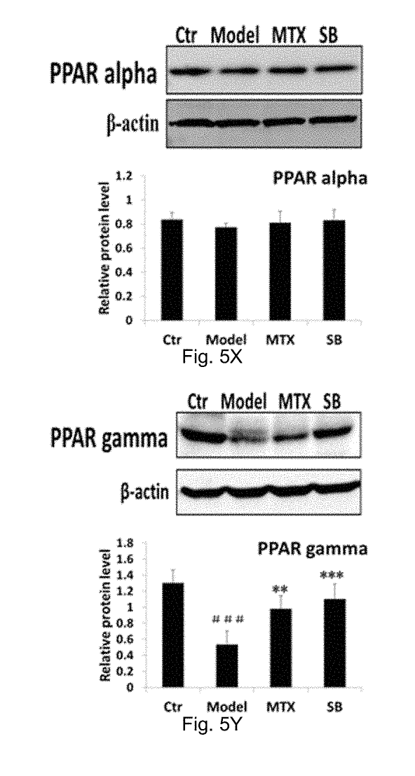

The real-time PCR result showed that the expression of Lipoprotein Lipase (LPL), Cholesterol 7-alpha-Hydroxylase (CYP7A1), Cholesterol 27-alpha-Hydroxylase (CYP27A1), Sterol Regulatory Element-Binding Protein1 (SREBP1), class B Type I Scavenger Receptor I (SR-BI), Low Density Lipoprotein Receptor (LDLR), Glucose-6-Phosphate Dehydrogenase (G6PD), Adipocyte Protein 2 (aP2), Cluster of Differentiation 36/Fatty Acid Translocase (CD36/FAT), Cytochrome P450 2E1 (CYP2E1), FAT/CD36 Liver X Receptor (LXR) and Farnesoid X Receptor (FXR) were elevated, and HMG-CoA reductase (HMGCR), Acyl Coenzyme A (CoA) Synthetase (ACS), Carnitine Palmitoyltransferase I (CPT1), Peroxisome Proliferator-Activated Receptor alpha (PPAR-a) and gamma (PPAR-γ) were reduced in the liver tissues of vehicle-treated arthritis rats as compared to normal rats (FIG. 5A to 5M), while Silybin treatments significantly modulated the lipid metabolism pathway.

Excessive fat accumulation in the liver can occur as a result of increased fat delivery, increased fat synthesis, reduced fat oxidation, and/or reduced fat export in the form of VLDL. Firstly, it has been examined how the lipid metabolism changes in the liver of the arthritis AIA group occurred preferentially with hepatic fat accumulation and reduced lipid levels. The level of gene expression of key lipogenic enzymes involved in the fatty acid synthesis and transport was significantly up-regulated, including G6PD, aP2 and CD36/FAT as shown in FIG. 5A to 5M, while the level of Acetyl-CoA Carboxylase 1 (ACC1) and FAS (FAS) did not change (data was not shown). The mRNA level of gene CPT-I and ACS which are the rate-limiting enzyme in FAs oxidation and FAs activation, respectively, were significantly decreased in the arthritis AIA group. Therefore, these results indicate an increased fatty acid synthesis with decreased catabolism in arthritis AIA model which were accumulated as lipid droplet in the liver.

Next, the level of gene expression of key lipogenic enzymes involved in lipid metabolism in the liver of arthritis AIA group has been compared with the normal and Silybin-treated group. There was a significant increase in the mRNA level of LPL, the rate-limiting enzyme in triglyceride hydrolysis to fatty acid, in arthritis AIA model group, which was reversed by Silybin treatment. But gene expression of ApoC-II and ApoE, the activator of LPL, did not change. The mRNA level of SR-BI and LDLR which are responsible for the uptake the lipoprotein particles into cells were both exhibited notably up regulation in the arthritis AIA model group compared with control group (FIG. 5A to 5M). Moreover, significant increase was noticed with the mRNA level of CYP7A1 and CYP27A1 which was the important enzyme for the catabolism and excretion of cholesterol in the liver. Based on these data, the arthritis AIA model has enhanced uptake of lipoprotein along with hydrolysis of lipoprotein, which could significantly increase lipid accumulation in the liver.

Moreover, the nuclear hormone receptors, including PPARs, LXRs and FXR who regulate the transcription of a large number of genes involved in multiple aspects of lipid and lipoprotein metabolism were examined. It has been found that mRNA levels of genes involving in hepatic lipogenesis transcription factor such as SREBP1, LXR, and FXR were markedly increased (p<0.001), while PPAR-a, and PPAR-γ were decreased (FIG. 5A to 5M). Treatment with Silybin or MTX attenuated these increased changes but not for the decreased gene expression levels of ACS, CPT-I and HMGCR as shown in FIG. 5A to 5M. These results indicated that hepatic lipogenesis transcription factor and its downstream enzymes involved in either in the de novo synthesis and transport of fatty acid, and in the uptake and hydrolysis of lipoprotein were altered in the arthritis AIA model, which were attenuated with the treatment of MTX and Silybin.

Following confirmation of differential gene expression of the key lipogenic enzymes involved in lipid metabolism, Western blot analysis was further conducted to verify the gene determination results (FIG. 5N to 5Y). Western blot analysis indicated that LPL, G6PD, CYP7A1, CYP27A1, aP2, CD36/FAT, SREBP1, LXR protein was up-regulated in the arthritis AIA model, whereas CYP2E1 and FXR protein levels did not change. In fact, the treatment of MTX and Silybin ameliorated changes of key lipogenic enzyme in both gene and protein levels (FIG. 5N to 5Y).

### Example 1B

MCD Diet Induced Mice NASH Model

Male wild-type (WT) mice C57Bl/6 were fed either MCS chow diet (Trophic Animal Feed High-tech Co., Ltd, China 20, #TP 3005GS); MCD diet (Trophic Animal Feed High-tech Co., Ltd, China, #TP 3005G) for 8 weeks. The respective composition is given in Tables 15 and 16. Animals were randomly divided into four groups (n=10): 1) controls, fed a standard control MCS diet; 2) rats fed a high-fat MCD diet; 3) rats fed the MCD diet treated with Silybin (150 mg/kg, oral) and 4) rats fed the MCD diet treated with Silybin (300 mg/kg, oral). Silybin was dissolved with solvent of 35% PEG400: 15% Cremophor EL: 5% ethanol: 45% saline. Body weight was intermittently monitored during the diet-induction period and every two or three days during the intervention period.

At the end of the 8 weeks, mice were sacrificed under ether anesthesia. The liver were immediately removed and weighed. A large portion of liver was snap-frozen in the liquid N₂ with remaining tissue was fixed in 10% buffer formalin, processed and embedded in paraffin for histological examination after H&E staining. For evaluation of steatohepatitis, the level of ALT, AST, TG, and TC were determined according to manufacturer's instructions with commercial kits. Moreover, histological assessment and scoring according to standardized criteria were carried out by a pathologist blinded to the study (Kleiner, D. E., et al., Hepatology, 2005, 41(6): p. 1313-21). The cytokines levels in serum were measured using commercially available ELISA kits including TNF-α, and IL-6 as well as SOD.

TABLE 15
Composition of MCD diet used for introducing NASH animal model
MCD diet composition

| | | | | | |
|---|---|---|---|---|---|
| Sucrose | 455.3 | mg | L-Isoleucine | 8.2 | mg |
| Corn Starch | 200.0 | mg | L-Leucine | 11.1 | mg |
| Corn Oil | 100.0 | mg | L-Lysine Hydrochloride | 18.0 | mg |
| Alphacel Non-Nutritive Bulk | 30.0 | mg | L-Phenylalanine | 7.5 | mg |
| AIN 76 Mineral Mix | 35.0 | mg | L-Proline | 3.5 | mg |
| Dicalcium Phosphate | 3.0 | mg | L-Serine | 3.5 | mg |
| L-Alanine | 3.5 | mg | L-Threonine | 8.2 | mg |
| L-Arginine Hydrochloride | 12.1 | mg | L-Tryptophan | 1.8 | mg |
| L-Asparagine Monohydrate | 6.0 | mg | L-Tyrosine | 5.0 | mg |
| L-Aspartic Acid | 3.5 | mg | L-Valine | 8.2 | mg |
| L-Cystine | 3.5 | mg | DL-alpha-Tocopherol Acetate (250 u/mg) | 0.484 | mg |
| L-Glutamic Acid | 40.0 | mg | Vitamin A Palmitate (250,000 u/mg) | 0.0792 | mg |
| Glycine | 23.3 | mg | Vitamin D3 (400,000 u/gm) | 0.0055 | mg |
| L-Histidine Hydrochloride | 4.5 | mg | Ethoxyquin | 0.02 | mg |
| Biotin | 0.0004 | mg | PyridoxineHydrochloride | 0.0220 | mg |
| D-Calcium Pantothenate | 0.0661 | mg | Riboflavin | 0.022 | mg |
| Folic Acid | 0.002 | mg | Thiamine Hydrochloride | 0.022 | mg |
| Inositol | 0.1101 | mg | Vitamin B12 (0.1% trit.) | 0.0297 | mg |
| Menadione | 0.0496 | mg | Ascorbic acid | 1.0166 | mg |
| Niacin | 0.0991 | mg | Corn Starch | 3.4503 | mg |
| p-Aminobenzoic Acid | 0.1101 | mg | | | |

TABLE 16
Composition of MCS diet in the control group
MCS diet Composition

| | | | | | |
|---|---|---|---|---|---|
| Sucrose | 455.3 | mg | L-Isoleucine | 8.2 | mg |
| Corn Starch | 200.0 | mg | L-Leucine | 11.1 | mg |
| Corn Oil | 100.0 | mg | L-Lysine Hydrochloride | 18.0 | mg |
| Alphacel Non-Nutritive Bulk | 30.0 | mg | L-Phenylalanine | 7.5 | mg |
| AIN 76 Mineral Mix | 35.0 | mg | L-Proline | 3.5 | mg |
| Dicalcium Phosphate | 3.0 | mg | L-Serine | 3.5 | mg |
| L-Alanine | 3.5 | mg | L-Threonine | 8.2 | mg |
| L-Arginine Hydrochloride | 12.1 | mg | L-Tryptophan | 1.8 | mg |
| L-Asparagine Monohydrate | 6.0 | mg | L-Tyrosine | 5.0 | mg |
| L-Aspartic Acid | 3.5 | mg | L-Valine | 8.2 | mg |
| Methionine | 3.3 | mg | Choline | 0.00001 | mg |
| L-Cystine | 3.5 | mg | DL-alpha-Tocopherol Acetate (250 u/mg) | 0.484 | mg |
| L-Glutamic Acid | 40.0 | mg | Vitamin A Palmitate (250,000 u/mg) | 0.0792 | mg |
| Glycine | 23.3 | mg | Vitamin D3 (400,000 u/gm) | 0.0055 | mg |
| L-Histidine Hydrochloride | 4.5 | mg | Ethoxyquin | 0.02 | mg |
| Biotin | 0.0004 | mg | Pyridoxine Hydrochloride | 0.0220 | mg |
| D-Calcium Pantothenate | 0.0661 | mg | Riboflavin | 0.022 | mg |
| Folic Acid | 0.002 | g | Thiamine Hydrochloride | 0.022 | mg |
| Inositol | 0.1101 | mg | Vitamin B12 (0.1% trit.) | 0.0297 | mg |
| Menadione | 0.0496 | mg | Ascorbic acid | 1.0166 | mg |
| Niacin | 0.0991 | mg | Corn Starch | 3.4503 | mg |
| p-Aminobenzoic Acid | 0.1101 | mg | | | |

Silybin Ameliorates Liver Injury of the MCD Diet-Induced NASH in Mice

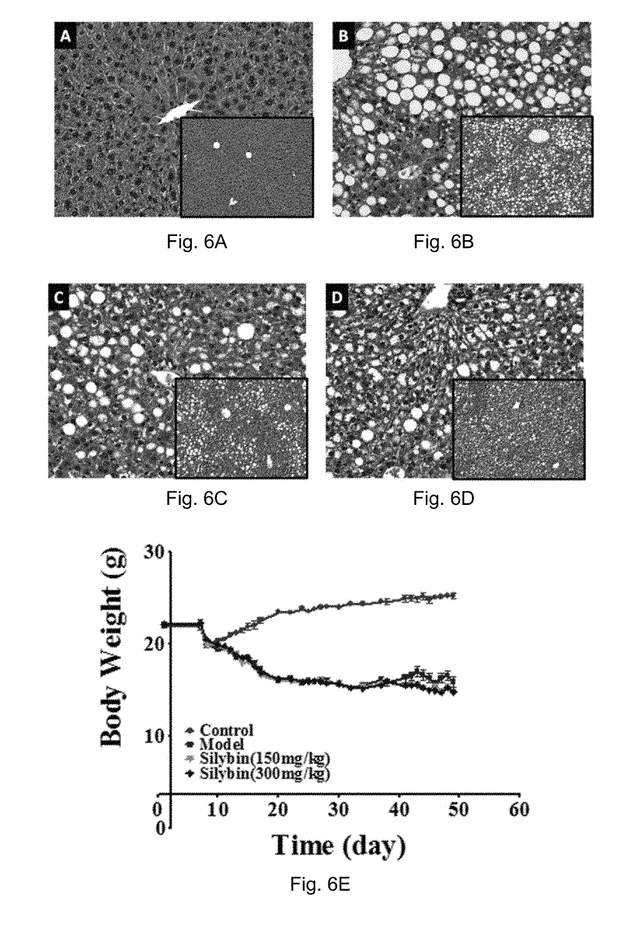

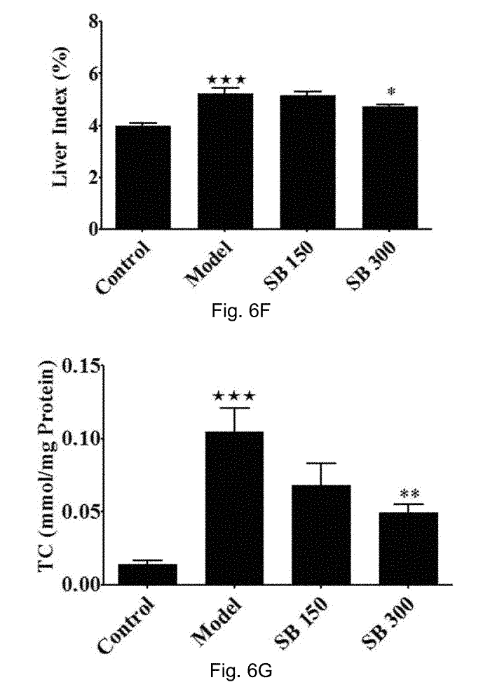

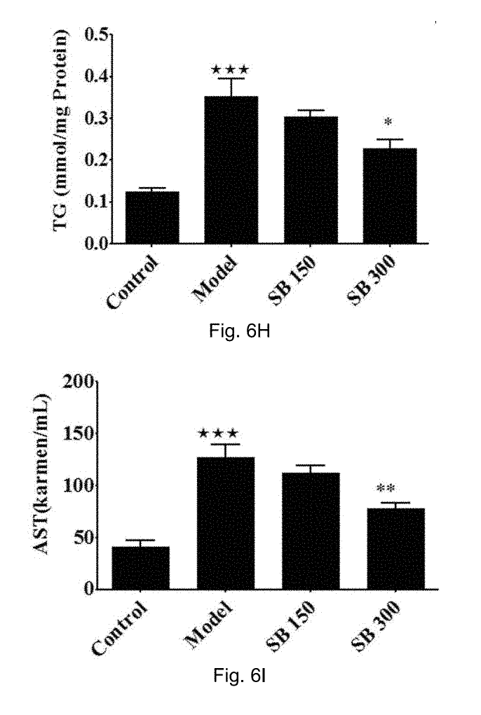

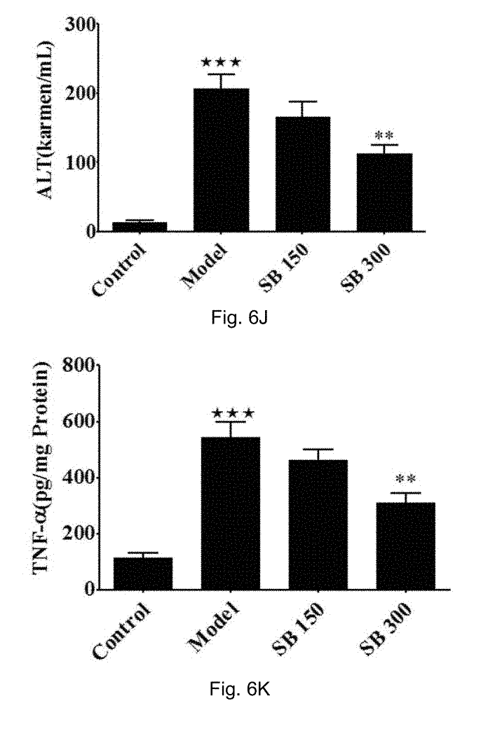

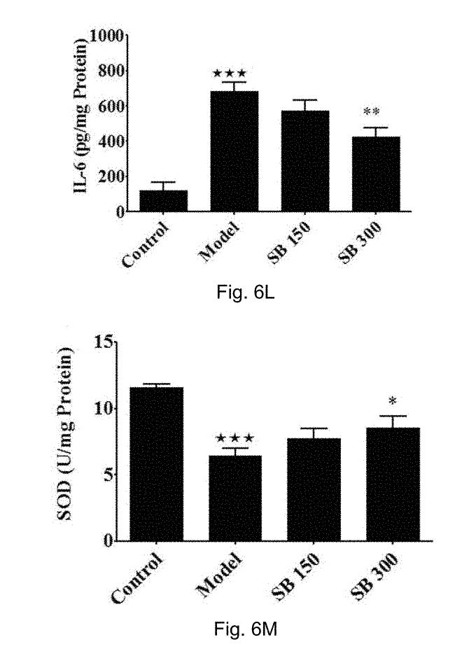

In order to evaluate whether lipid metabolism modulation effect of Silybin contributes to its liver protection effect, the lipids profile as well as therapeutic effect in a MCD-diet induced mice NASH model has been further evaluated. As MCD diet better models the pathobiological mechanisms that cause human NAFLD to progress to advanced NASH (Machado M. V., et al., PLoS ONE 2015, 10(5): p. e0127991), thus the MCD died induced NASH model has been used to study the effects of Silybin on risk factors including cytokines, oxidative stress and lipid metabolism which are the common factors in the pathogenesis of both RA and NASH. As shown in FIG. 6E, eight weeks of MCD diet feeding dramatically decreased body weight and liver weight but increased liver/body weight ratio (P<0.001), while treatment with Silybin at 300 mg/kg had no effect on body weight but caused a moderate decrease in the liver/body weight ratio (P<0.05). Histological analysis of liver specimens stained with H&E from controls and mice on the MCD diet or on the MCD diet with Silybin treatment for 8 weeks is shown in FIG. 6A to 6D and Table 17. The MCD diet group showed micro- and macro-vesicular steatosis, indicative of disturbed lipid metabolism, with inflammation and ballooning degeneration (FIG. 6B); mice treated with Silybin exhibited lower scores of steatosis, inflammation and ballooning (FIGS. 6C and 6D) (P<0.01).

TABLE 17
Effects of Silybin on histopathological
changes of MCD diet induced NASH mice

| | Control | Model | SB 150 | SB 300 |
|---|---|---|---|---|
| Steatosis | 0 | 1.80 ± 0.13 | 1.00 ± 0* | 0.92 ± 0.13* |
| Ballooning | 0 | 1.93 ± 0.07 | 0.96 ± 0.04* | 0.83 ± 0.09** |
| Inflammation | 0 | 2.47 ± 0.08 | 0.92 ± 0.10** | 0.88 ± 0.12** |

*p < 0.05, **p < 0.01, ***p < 0.001 versus normal rats or versus the vehicle-treated rats (n = 6-8)

The MCD diet induced NASH in C57BL/6 mice resulted in markedly increased serum TC, TG, ALT, AST and liver TNF-α, IL-6 levels as well as decreased SOD levels (P<0.001) after 8 weeks administration compared with those of controls, indicating considerable hepatocellular injury, inflammation and lipoperoxidation. With Silybin treatment, serum transaminase, cytokines levels, lipid profiles and oxidative stress levels were significantly restored in comparison with vehicle treated MCD diet model group (FIG. 6F to 6M).

Lipid-Associated Metabolites Play a Critical Role in the Pathogenesis of NASH and the Therapeutic Effect of Silybin on the NASH Model

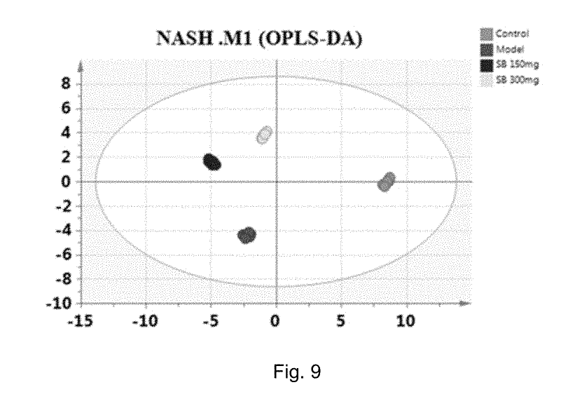

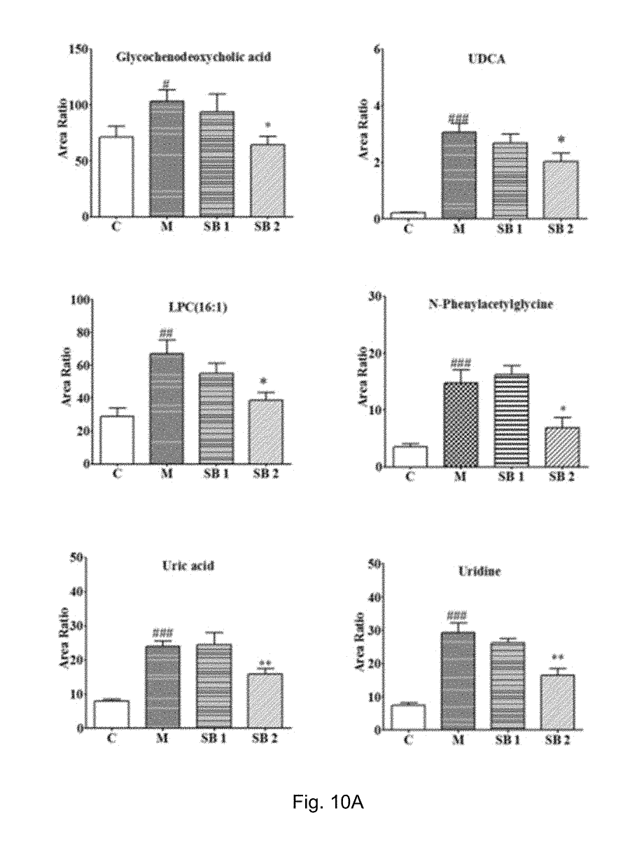

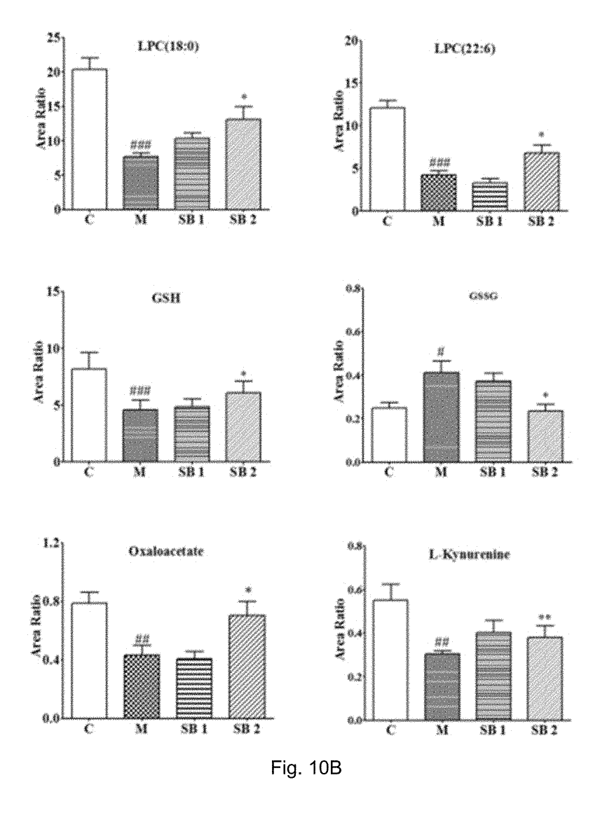

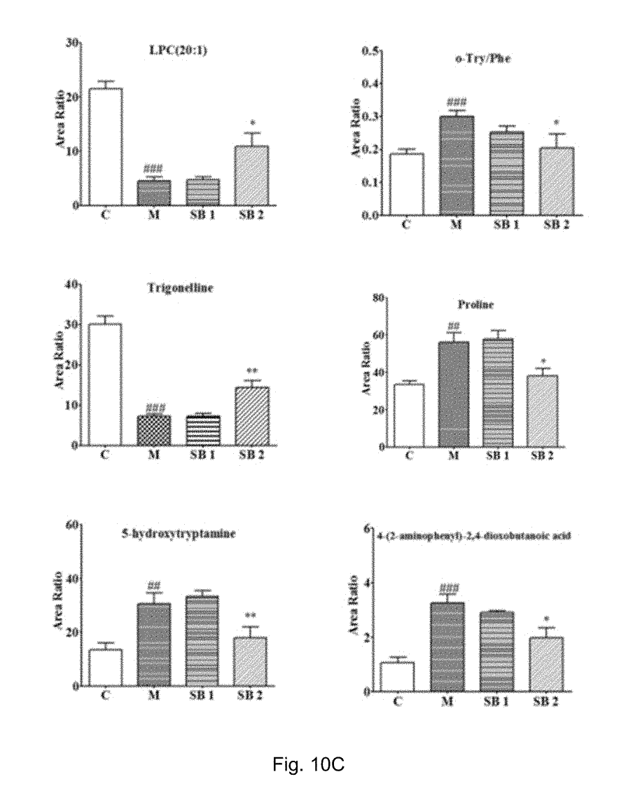

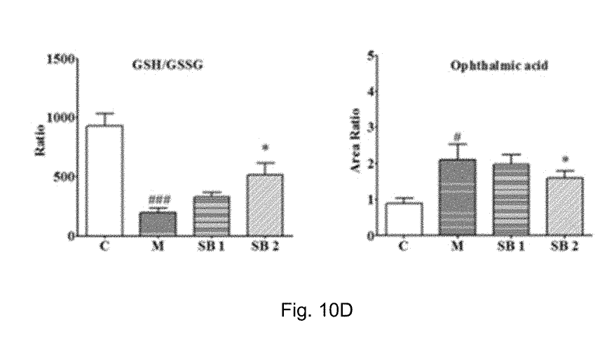

Plasma samples of MCD-diet model were analyzed by LC-MS/MS for better understanding the functional metabolism in NASH model as well as the mechanism of Silybin treatment. Within the initial cohort, PCA revealed a clear separation of the plasma metabolomes from control, vehicle-treated MCD diet induced NASH mice and Silybin treated NASH mice. OPLS-DA (R²X=0.877, Q²=0.749) was also used to identify and rank signature metabolites explaining most of the variance of metabolomes among the control, model and treatment group based on VIP scores and/or P value (FIG. 9). Unsupervised hierarchical clustering of this metabolite set classified NASH vs. control mice with 100% accuracy in both cohorts. A 15-metabolite signature with top-ranked VIP scores (VIP>1.0) separated control, model and Silybin treatment groups (p<0.05) was identified in Table 18 that distinguish each groups. The majority of these fifteen metabolites include bile acids (GCDCA, UDCA), lipids (LPC(18:0), LPC(22:6), LPC(20:1), LPC(16:1)), fatty acid (N-phenylacetylglycine), TCA cycle metabolite (oxaloacetate), amino acids (L-kynurenine, 5-hydroxytryptamine) and purine nucleotide metabolites (uric acid, uridine, trigonelline), indicating that many metabolic alterations also occur in NASH disease as shown in FIG. 10A to 10D.

TABLE 18
Classification of metabolites identified according to metabolic pathway and
comparison of relative levels of metabolites. (n = 6-8, P < 0.05 and VIP > 1)

| | | Model VS Control | | SB300 VS Model |
|---|---|---|---|---|
| Metabolite(NASH) | Physiological metabolism | VIP | Trend | Trend |
| Glycochenodeoxycholic | Bile acid metabolism | 1.05 | ↑* | ↓* |
| acid(GCDCA) | | | | |
| Ursodeoxycholic acid (UDCA) | Bile acid metabolism | 1.02 | ↑*** | ↓* |
| | Glycerophospholipid | | | |
| LPC(18:0) | metabolites | 1.00 | ↓** | ↑* |
| | Glycerophospholipid | | | |
| LPC(22:6) | metabolites | 1.11 | ↓*** | ↑* |
| | Glycerophospholipid | | | |
| LPC(20:1) | metabolites | 1.00 | ↓*** | ↑* |
| | Glycerophospholipid | | | |
| LPC(16:1) | metabolites | 1.18 | ↑** | ↓* |
| N-Phenylacetylglycine | Fatty acid metabolism | 1.10 | ↑*** | ↓* |
| GSH | Glutathione metabolism | 1.11 | ↓*** | ↑* |
| GSSG | Glutathione metabolism | 1.02 | ↑* | ↓* |
| | Purine nucleotide | | | |
| Trigonelline | metabolism | 1.05 | ↓*** | ↑** |
| | Purine nucleotide | | | |
| Uric acid | metabolism | 1.01 | ↑*** | ↓** |
| | Purine nucleotide | | | |
| Uridine | metabolism | 1.04 | ↑*** | ↓** |
| Oxaloacetate | Citric acid cycle | 1.05 | ↓** | ↑* |
| L-Kynurenine | Amino acid metabolism | 1.03 | ↓** | ↑** |
| 5-hydroxytryptamine | Amino acid metabolism | 1.06 | ↑** | ↓** |
| GSH/GSSG | Glutathione metabolism | 1.04 | ↓*** | ↑* |
| Malondialdehyde (MDA) | Lipid peroxidation | 0.94 | ↑*** | ↓** |
| Proline | Amino acid metabolism | 0.99 | ↑** | ↓* |
| 4-(2-aminophenyl)-2,4- | Amino acid metabolism | 0.97 | ↑*** | ↓* |
| dioxobutanoic acid | | | | |
| Ophthalmic acid | Amino acid metabolism | 0.87 | ↑* | ↓* |

* P-Values were determined using the Student's t test, * P < 0.05, ** P < 0.01 and *** P < 0.01 VS model or Control

Given the prevalence of fatty liver diseases in RA populations and the association between chronic inflammation and increased rates of lipodystrophy, hepatic steatosis, and insulin resistance, the plasma metabolome markers of AIA have been compared with MCD diet induced NASH model. Significant overlap was found between the two data sets, with common perturbed groups of metabolites including lysophosphatidylcholine (LPC), bile acids (BAs), amino acid (AA), nucleic acid and citrate acid cycle metabolites. The majority of lipid metabolites altered in RA and NASH models including LPC (16:1), LPC (22:6), LPC (18:0), and GCDCA. Elevated serum bile acids have been strongly related to liver disease in a number of recent studies. However, it is the first time to find that bile acids were significantly increased in arthritis AIA model and correlated with the therapeutical treatment. Collectively, these alterations show substantial overlap with those previously reported in NAFLD and NASH, (i.e., bile acids, LPC, FA, uric acid), raising the possibility that mechanisms underlying development of NAFLD (i.e., lipid accumulation, lipid peroxidation and mitochondrial dysfunction) may also contribute to liver abnormality in RA patients.

GSH/GSSG, uric acid and uridine levels were also significantly higher in both models (FIG. 4 and FIG. 10), which are the biomarkers of oxidative stress. It is well known that increased oxidative stress is a widely associated the pathogenesis of NASH and RA. Moreover, significant increase amino acids such as o-tyr/phe, and L-kynurenine were found in the both RA and NASH animal model samples (FIG. 4 and FIG. 10). Many researches showed that metabolism of tryptophan through the kynurenine pathway were related to immune system and inflammation. On the other side, it is known that the kynurenine pathway of tryptophan degradation regulates lipid metabolism. Thus, the changes of kynurenine pathway may be one of the reasons that liver lipid profile changed in response with the autoimmune disorder and inflammation in the liver of both RA and NASH model, while the treatment of Silybin modulate the kynurenine pathway.

TCA cycle related metabolites such as citric acid, oxaloacetate, and succinic acid were also altered in the AIA arthritis model compared with controls, while similar changes were noticed in the MCD diet induced NASH model. It has been reported that higher liver fat is associated with more active TCA cycle metabolism, which was consistent with the observation in this study. All these indicated that lipid accumulation happened in the liver of both RA and NASH models was closely related with the pathogenesis, which was reflected with the metabolites changes in the metabolomic investigations.

Discussion of the Results

There are strong evidences supporting that metabolites are important players in biological systems and that inflammation and diseases cause the disruption of metabolism pathways. Changes in lipid profiles in the blood of RA patients called “lipid paradox” have been widely observed, in which decreased TC, LDL-C and HDL-C levels were not only associated with active inflammation in RA patients, but also with higher rather than lower risk of cardiovascular disease. This altered lipid pattern in RA is mirrored in sepsis, cancer and other inflammation states suggesting inflammation is associate with the lipid levels, which is line with the observation that suppressing inflammation by DMARDs and biological agents could improve the levels of lipids. However, the molecular mechanisms behind the dyslipidemia as well as its relationship with pathogenesis as well as treatment in RA are far from clear.

Metabolomic techniques were used to analyze the primary changes in metabolite profile in both the arthritis AIA model and the MCD diet induced NASH model in the presence or absence treatment of Silybin. Significant overlap was found between the two data sets, with common perturbed groups of compounds including free fatty acids (FFAs), lysophosphatidylcholine (LPC), bile acids (BAs), and amine acid (AA), which indicate that multiple metabolism pathways changed in these two diseases model.

Based on the metabolomic analysis results, the major metabolism change happened with lipid profile in the arthritis AIA model and the MCD diet model, suggesting altered lipid metabolism may be shared by fatty liver and RA. In order to explore the underlying mechanisms for the changes of lipid profile, the levels of key lipogenic lipid metabolism enzymes has been evaluated. The real-time PCR result showed that the expression of LPL, CYP7A1, LDLR, SREBP1, SR-BI, G6PD, LXR and FXR were elevated, and HMG-COA, PPAR-α and PPAR-γ were reduced in the liver tissues of AIA rats. These results were strongly consistent with the protein results obtained by Western blot. Gene expression and protein level analyses of key enzymes in the lipid metabolism further delineate the mechanistic insights on how altered lipid metabolism related with the pathogenesis of RA and treatment.

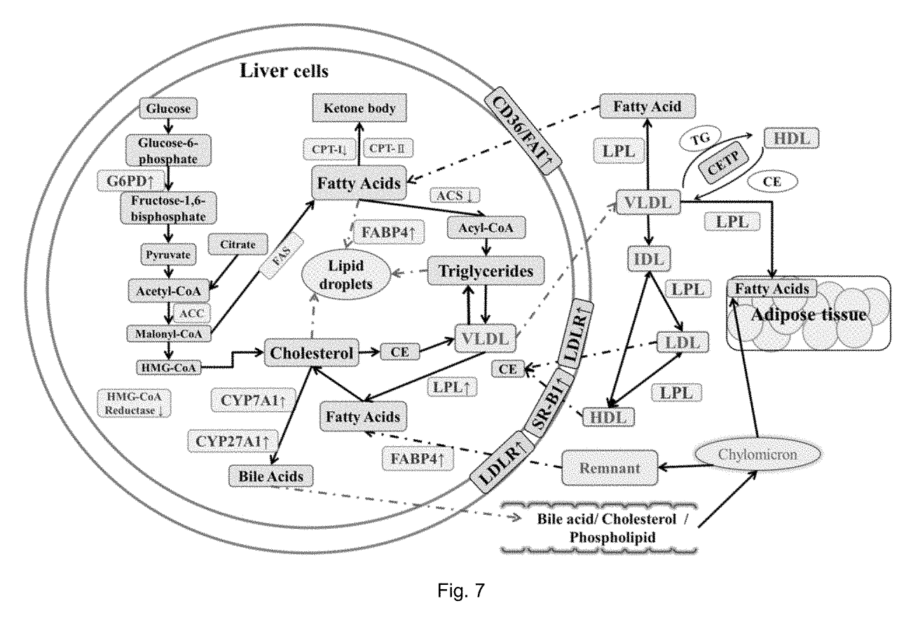

Liver fatty acids (FA), which are believed to be the more active and injury-inducing lipids, were higher in the MCD-diet group and arthritis AIA group. LPL levels were significantly increased in the arthritis AIA model, which may be partly explained the mechanisms driving liver fat accumulation in liver as LPL is the major enzymes hydrolyzing triglycerides in lipoprotein into free fatty acids and glycerol as shown in FIG. 7. LPL is normally not expressed in the liver of adult humans and animals, but higher LPL activity has been observed in the liver of obese patients than controls which contributed to the typical steatosis observed in these patients. In agreement with the previous studies, the data showed that liver of arthritis AIA rats presents relatively large quantities of LPL by comparing with the normal and treated rats. Moreover, unlike the controls, this enzyme could be synthesized in the liver because it also presents LPL mRNA. Thus, The tissue-specific overexpression of LPL in liver increased cellular stores of TG and probably other lipids and favored the steatosis observed in patient with RA.

Moreover, arthritis AIA caused up-regulation of protein for fatty acid de novo lipogenesis like G6PD and transport proteins such as aP2 and CD-36/FAT (FIG. 7). The pathogenic role of CD36 in hepatic steatosis is well defined and disruption of CD36 attenuates the fatty liver. Although G6PD is required for lipogenesis, the role of G6PD is poorly understood in fatty liver disease as well as other chronic diseases. Park et al. reported that in adipocyte, G6PD overexpression stimulated the expression of most adipocyte marker gene and elevated the levels of cellular free fatty acid and TG (Park, J., et al., Molecular and Cellular Biology, 2005, 25(12): p. 5146-5157). Therefore, the overexpression of both G6PD and CD36 may partly contribute to the mechanisms of action on metabolic and inflammatory pathways in RA. In addition, adipocyte fatty acid binding protein (FABP4/aP2) has been shown a central role in fatty-acid import, storage and export as well as cholesterol and phospholipid metabolism. Recent studies demonstrated that loss of FABP4/aP2 could reduce macrophage inflammation and highlighted their considerable potential as therapeutic targets for a range of associated disorders such as obesity, diabetes and atherosclerosis. The level of aP2 in the liver in the arthritis AIA model was significantly increased and the expression was reduced with the treatment of MTX and Silybin. Thus, aP2 could be a potential therapeutic target for RA.

In the hepatocyte, fatty acids undergo oxidation or esterification with glycerol and cholesterol to form TG and cholesteryl esters (CE), respectively. The gene levels of CPT-I and ACS which are the rate-limiting enzymes in FAs oxidation and FAs activation, were significantly decreased in arthritis AIA, while ACC1, a key enzyme for TG synthesis, did not change. Therefore, accumulation of fatty acid could further lead to the accumulation of TG and CE in the liver instead of oxidation (FIG. 7). Moreover, cholesterol uptake is mainly mediated by the high density lipoprotein receptor SR-BI and the low density lipoprotein receptor LDLR which were both exhibited notably up regulation in arthritis AIA model group compared with the control group. Hepatic overexpression of the SR-BI and LDLR significantly increased HDL-C, LDL-C and VLDL-triglyceride (Wiersma, H., et al., Journal of Lipid Research, 2010, 51(3): p. 544-553), and promote selective uptake of cholesterol from the circulation by the liver, which could lead to the fatty liver as well as reduced cholesterol in the plasma of RA patient during active stages. A significant enhanced level of CYP7A1 and CYP27A1 has been observed here, which coincided with the elevated serum bile acids in arthritis AIA model and correlated with the therapeutical treatment for RA. Overexpression of the rate-limiting bile acid synthetic enzyme CYP7A1 and CYP27A1 results in bile acids accumulation in the liver.

Increased expression of lipogenic enzymes can be triggered by multiple mechanisms including modulated by several specific transcription factors. Activation of LXR-α has been shown to induce massive liver steatosis with up regulation the target genes of LPL, CYP7A1 and SREBP1 (Grefhorst, A., et al., Journal of Biological Chemistry, 2002, 277(37): p. 34182-34190). The activation of LXR observed here could be the mechanism by which the liver fatty acid synthesis increased and hepatic steatosis developed in patient of RA. Moreover, LXR pathway has been reported as the most upregulated pathway in RA synovial macrophages and activation of LXRs significantly enhanced Toll-like receptor (TLR)-driven cytokine secretion (Asquith, D. L., et al., Annals of the Rheumatic Diseases, 2013, 72(12): p. 2024-2031). Therefore, based on the present study, LXR is one of the major links between the inflammation and lipid metabolism disorder in RA. Moreover, treatment with MTX and Silybin greatly repressed the mRNA and/or protein expression of LPL, CYP7A1, and SREBP1 in liver of arthritis AIA model. SREBP1 are also transcription factors that were first identified in mammalian cells as key regulator of cellular lipid levels (Horton, J. D., Jet al., J Clin Invest, 2002, 109(9): p. 1125-31). However, the overexpression of SREBP1 observed in AIA rats was not modulated by the treatment of MTX and Silybin. SREBP1 positively regulates the expression of genes involved in cholesterol and fatty acid synthesis including fatty acid synthase (FAS) and HMG CoA reductase (HMGCR) which do not respond to the treatment in the present study. Thus, SREBP-1 may partly relate with the changes of lipid metabolism in AIA model but not the major pathway. FXR, another important nuclear receptor regulating lipid metabolism, regulates bile acid uptake into hepatocytes and bile acid biosynthesis as well as fatty acid β-oxidation. However, given the elevated bile acid, FXR level did not change in the arthritis AIA model indicating that it may not involve in the pathogenesis of RA.

Similar as LXR, the nuclear receptor family of PPAR has emerged as important regulator of metabolic, inflammatory and immunity signaling (Hong, C. and Tontonoz, P., Curr Opin Genet Dev, 2008, 18(5): p. 461-7) and comprised of three related proteins, PPARα, PPARβ/δ, and PPARγ. PPARγ transcriptional regulates genes involved in lipid metabolism, and energetics, including aP2, G6PD, CD36 and LXRα, while recently studies suggest that PPARγ is an important immunmodulator and suppresses the production of inflammatory cytokines as well as the pro-inflammatory gene expression (Shahin, D., et al., Clin Med Insights Arthritis Musculoskelet Disord, 2011, 4: p. 1-10). Wu et al. found that overexpression of PPARγ delivered by Ad-PPARγ could redistribute the fatty acid from liver to adipose tissue by enhance the expression of fatty acid uptake genes (aP2, LPL, CD36 and SREBP1) in adipose tissue and to a lesser extent in liver (Wu, C. W., et al., Gene Therapy, 2010, 17(6): p. 790-798). Interestingly, the present study made the opposite observation that hepatic levels of PPARγ mRNA and protein were specifically decreased in arthritis AIA rats, while hepatic mRNA levels of targets CD36, G6PD and aP2 were significantly increased. Therefore, PPARγ could be important for modulation the lipid metabolism and leading to steatosis. Further, down regulation of PPARα and its target genes CPT-1 has been found, which would direct fatty acids taken up towards storage instead of the β-oxidation pathway.

Drug-induced liver injury is frequent in RA, especially during nonsteroidal anti-inflammatory drug (NSAID) and methotrexate treatments, which is significantly more frequent than primary disease-related liver involvement. Considering that a wide spectrum of rheumatic diseases can affect the liver with various degrees, there is a strong need for a candidate compound which could work on both arthritis joint and abnormal liver in RA patient. Significantly reduction in liver lipids correlated with an improvement in the inflammatory markers was observed with the treatment of Silybin for the first time here. The fatty liver protection effect as well as lipid modulation of Silybin were further demonstrated in MCD diet induced NASH model.

In summary, the inventors have shown that the acute model of AIA transiently alters the metabolism, especially the lipid profiles in the liver. The investigation of the mechanisms that leads to metabolic alterations ruled out an important participation of the lipid and lipoprotein metabolism enzymes including LXR, LPL, G6PD, aP2, CD36 and CYP7A1 etc. As the changes of these enzymes are opposite to those in the infection and inflammation induced acute phase response, it could partly explain the phenomenon of “lipid paradox” in patient of RA. Upregulated expression of these enzymes in the liver is expected to represent a characteristic feature of RA patients. Treatment of the disease with Silybin lead to a reduction in inflammation and modulation in lipid profile towards normal as well as its protect effect on liver function. Oral administration of Silybin significantly reduced swelling, hind paw bone erosion, inflammation and liver injury without appearing toxicity in AIA arthritis and MCD-diet induced NASH model, indicating that it is a potential treatment for arthritis with protection of liver function.

1. A method for reducing abnormalities in lipid metabolism and for reducing inflammation in a subject comprising administering an effective amount of a flavonolignan to said subject, wherein the subject suffers from an autoimmune inflammatory disease accompanied by abnormalities in lipid metabolism; wherein the flavonolignan decreases two or more of elevated lipid metabolism markers in liver tissue or liver cells in the subject compared to untreated subjects, and wherein the lipid metabolism markers are selected from Lipoprotein Lipase, Adipocyte Protein 2, Cholesterol 7-Alpha-Hydroxylase, Sterol 27-Hydroxylase, Glucose-6-Phosphate Dehydrogenase, Fatty Acid Translocase, Sterol Regulatory Element-Binding Protein 1, Low-Density Lipoprotein (LDL) Receptor, Scavenger Receptor Class B member 1 (SR-B1) and Liver X Receptor alpha.

2. The method of claim 1, wherein the autoimmune inflammatory disease is an autoimmune arthritis and the subject is a mammal.

3. The method of claim 1, wherein the autoimmune inflammatory disease is rheumatoid arthritis and the abnormalities in lipid metabolism further include a level of high-density lipoprotein (HDL) cholesterol deviating from a reference value in healthy subjects.

4. The method of claim 1, wherein the abnormalities in lipid metabolism further include a decreased level of high-density lipoprotein (HDL) cholesterol, a normal or mildly increased level of total cholesterol (TC), a normal or mildly increased level of low-density lipoprotein (LDL) cholesterol and a normal or mildly increased level of triglycerides compared to a reference value in healthy subjects.

5. (canceled)

6. The method of claim 1, wherein reducing abnormalities in lipid metabolism includes one or more of an increase in the level of HDL cholesterol, a decrease of triglycerides and a decrease in the level of free fatty acids compared to untreated subjects.

7. The method of claim 1, wherein the subject further suffers from a liver disease.

8. The method of claim 7, wherein the liver disease is a fatty liver disease.

9. The method of claim 1, wherein the subject further suffers from a metabolic syndrome.

10. The method of claim 1, wherein reducing inflammation includes reducing elevated inflammation markers selected from two or more of Tumor Necrosis Factor alpha, Interleukin-1β, Prostaglandin E2, Matrix Metallopeptidase 9, TIMP Metalloproteinase Inhibitor 1, and the Erythrocyte Sedimentation Rate compared to untreated subjects.

11. The method of claim 1, wherein the subject further has hepatic abnormalities including an increased level of Alkaline Phosphatase (ALP) and Aspartate Aminotransferase (AST) compared to a reference value in healthy subjects and the method further comprises reducing hepatic abnormalities including reducing the level of Alkaline Phosphatase and Aspartate Aminotransferase compared to untreated subjects.

12. The method of claim 1, wherein the flavonolignan comprises one or more of:
Silybin A having Formula (Ia): 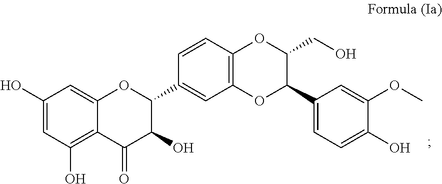 [SMILES: COc1cc([C@H]2Oc3cc([C@H]4Oc5cc(O)cc(O)c5C(=O)[C@@H]4O)ccc3O[C@@H]2CO)ccc1O]
and
Silybin B having Formula (Ib) 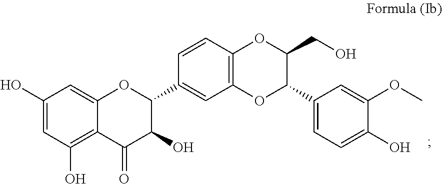 [SMILES: COc1cc([C@@H]2Oc3cc([C@H]4Oc5cc(O)cc(O)c5C(=O)[C@@H]4O)ccc3O[C@H]2CO)ccc1O]
or glycosides, salts or solvates thereof.

13. The method of claim 12, wherein the flavonolignan further comprises one or more of:
Isosilybin A having Formula (Ha): 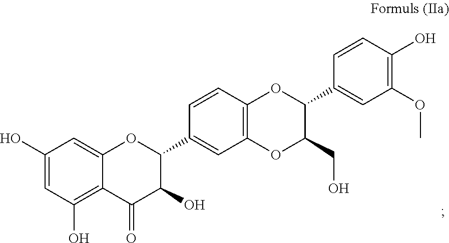 [SMILES: COc1cc([C@H]2Oc3ccc([C@H]4Oc5cc(O)cc(O)c5C(=O)[C@@H]4O)cc3O[C@@H]2CO)ccc1O]
Isosilybin B having Formula (IIb): 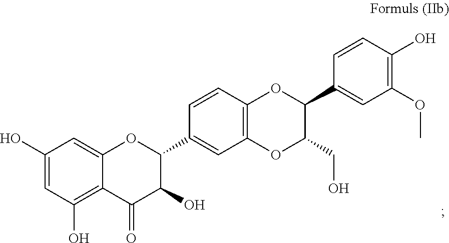 [SMILES: COc1cc([C@@H]2Oc3ccc([C@H]4Oc5cc(O)cc(O)c5C(=O)[C@@H]4O)cc3O[C@H]2CO)ccc1O]
Silychristin having Formula (Ma): 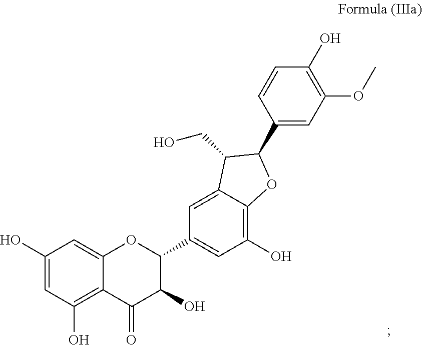 [SMILES: COc1cc([C@H]2Oc3c(O)cc([C@H]4Oc5cc(O)cc(O)c5C(=O)[C@@H]4O)cc3[C@@H]2CO)ccc1O]
and
Silydianin having Formula (IVa): 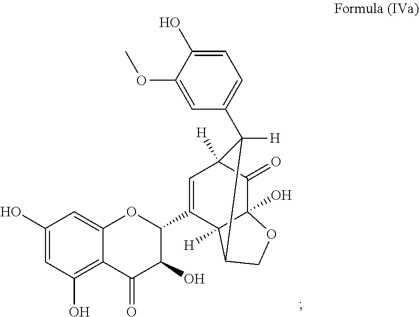 [SMILES: COc1cc(C2C3CO[C@]4(O)C(=O)[C@H]2C=C([C@H]2Oc5cc(O)cc(O)c5C(=O)[C@@H]2O)[C@H]34)ccc1O]
or glycosides, salts or solvates thereof.

14. The method of claim 12, wherein the flavonolignan is:
Silybin A (i.e. a compound of Formula (Ia)) or a glycoside, salt or solvate thereof;
Silybin B (i.e. a compound of Formula (Ib)) or a glycoside, salt or solvate thereof;
or a mixture of both, and is administered to the subject by an oral route.

15. A method for reducing the risk of a cardiovascular disease in a subject comprising administering an effective amount of a flavonolignan to said subject, wherein the subject suffers from an autoimmune inflammatory disease accompanied by abnormalities in lipid metabolism; the flavonolignan decreases two or more of elevated lipid metabolism markers in liver tissue or liver cells in the subject compared to untreated subjects, and wherein the lipid metabolism markers are selected from Lipoprotein Lipase, Adipocyte Protein 2, Cholesterol 7-Alpha-Hydroxylase, Sterol 27-Hydroxylase, Glucose-6-Phosphate Dehydrogenase, Fatty Acid Translocase, Sterol Regulatory Element-Binding Protein 1, Low-Density Lipoprotein (LDL) Receptor, Scavenger Receptor Class B member 1 (SR-B1) and Liver X Receptor alpha.

16. The method of claim 15, wherein the autoimmune inflammatory disease is rheumatoid arthritis and the abnormalities in lipid metabolism include a decreased level of HDL cholesterol compared to a reference value in healthy subjects.

17. The method of claim 15, wherein the subject further suffers from a metabolic syndrome.

18. The method of claim 15, wherein the flavonolignan is:
Silybin A having Formula (Ia): 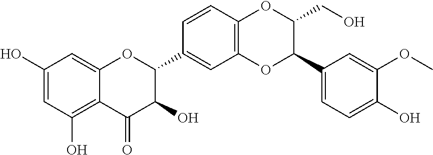 [SMILES: COc1cc([C@H]2Oc3cc([C@H]4Oc5cc(O)cc(O)c5C(=O)[C@@H]4O)ccc3O[C@@H]2CO)ccc1O]
Formula (Ia), including glycosides, a salt or solvate thereof; and
Silybin B having Formula (Ib): 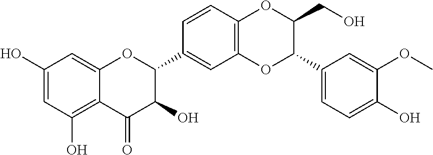 [SMILES: COc1cc([C@@H]2Oc3cc([C@H]4Oc5cc(O)cc(O)c5C(=O)[C@@H]4O)ccc3O[C@H]2CO)ccc1O]
Formula (Ib), including glycosides, a salt or solvate thereof;
or a mixture of both and is administered to the subject by an oral route.

19.

20.

21. The method of claim 1, wherein the subject further suffers from at least one of diabetes mellitus, obesity, and hypertension.

22. The method of claim 15, wherein the subject further suffers from at least one of diabetes mellitus, obesity, and hypertension.


SEQUENCE LISTING (58 sequences)

SEQ ID NO: 1 - DNA, 20 bp, Artificial Sequence
  synthezized
tagagatcggaaccgtgggt

SEQ ID NO: 2 - DNA, 20 bp, Artificial Sequence
  synthesized
gccccttgaacacctagcat

SEQ ID NO: 3 - DNA, 20 bp, Artificial Sequence
  sythesized
gcgtggtttccaacatgacc

SEQ ID NO: 4 - DNA, 20 bp, Artificial Sequence
  synthesized
tcctttgccactggaacctg

SEQ ID NO: 5 - DNA, 20 bp, Artificial Sequence
  synthesized
aagtttgccagttggggtca

SEQ ID NO: 6 - DNA, 20 bp, Artificial Sequence
  synthesized
gaagcatccgtcttcaccga

SEQ ID NO: 7 - DNA, 21 bp, Artificial Sequence
  synthesized
tttctcctgactctgcaacgg

SEQ ID NO: 8 - DNA, 20 bp, Artificial Sequence
  synthesized
gaggccttgtccccacatac

SEQ ID NO: 9 - DNA, 20 bp, Artificial Sequence
  synthesized
ttcgaaagagcggcatctcc

SEQ ID NO: 10 - DNA, 20 bp, Artificial Sequence
  synthesized
taggacatcgagcagaggct

SEQ ID NO: 11 - DNA, 20 bp, Artificial Sequence
  synthesized
ttcgaacagagcgggatgat

SEQ ID NO: 12 - DNA, 20 bp, Artificial Sequence
  synthesized
ccttatcctgcgagcccttt

SEQ ID NO: 13 - DNA, 21 bp, Artificial Sequence
  synthesized
aagaagtcgggctgacactgg

SEQ ID NO: 14 - DNA, 21 bp, Artificial Sequence
  synthesized
gaggacatgctatcggccatt

SEQ ID NO: 15 - DNA, 21 bp, Artificial Sequence
  synthesized
cttctgcgaaggcatttggac

SEQ ID NO: 16 - DNA, 20 bp, Artificial Sequence
  synthesized
ggcatacatcccttccgtga

SEQ ID NO: 17 - DNA, 20 bp, Artificial Sequence
  synthesized
agacccagagccatcgtagt

SEQ ID NO: 18 - DNA, 21 bp, Artificial Sequence
  synthesized
ggccactgggaagatctagtg

SEQ ID NO: 19 - DNA, 20 bp, Artificial Sequence
  synthesized
ctggcctccctgatgaataa

SEQ ID NO: 20 - DNA, 20 bp, Artificial Sequence
  synthesized
ggcggtctccactgagaata

SEQ ID NO: 21 - DNA, 21 bp, Artificial Sequence
  synthesized
gaggagttctttgcccgtaac

SEQ ID NO: 22 - DNA, 22 bp, Artificial Sequence
  synthesized
atctctttgcccaggtagtggt

SEQ ID NO: 23 - DNA, 20 bp, Artificial Sequence
  synthesized
caggtgtcaaatgatggccc

SEQ ID NO: 24 - DNA, 20 bp, Artificial Sequence
  synthesized
agtaagtgaagcacccctgc

SEQ ID NO: 25 - DNA, 20 bp, Artificial Sequence
  synthesized
tcgtcatccggtcagagagt

SEQ ID NO: 26 - DNA, 20 bp, Artificial Sequence
  synthesized
acacattccaccaccagctt

SEQ ID NO: 27 - DNA, 20 bp, Artificial Sequence
  synthesized
ggaggttgtctacgagccag

SEQ ID NO: 28 - DNA, 20 bp, Artificial Sequence
  synthesized
caaagcggtgtgagtctgtc

SEQ ID NO: 29 - DNA, 22 bp, Artificial Sequence
  synthesized
cttggatgtggaacccataact

SEQ ID NO: 30 - DNA, 22 bp, Artificial Sequence
  synthesized
cgatggtcccagtctcatttag

SEQ ID NO: 31 - DNA, 21 bp, Artificial Sequence
  synthesized
accctcatctctgaccatact

SEQ ID NO: 32 - DNA, 21 bp, Artificial Sequence
  synthesized
gtcttgggtgtagggtgattt

SEQ ID NO: 33 - DNA, 22 bp, Artificial Sequence
  synthesized
ggtcacatggtaagagggtatg

SEQ ID NO: 34 - DNA, 22 bp, Artificial Sequence
  synthesized
ggaggtagaagtaggtggatct

SEQ ID NO: 35 - DNA, 24 bp, Artificial Sequence
  synthesized
tggaaggaaggtagagtaggtggg

SEQ ID NO: 36 - DNA, 24 bp, Artificial Sequence
  synthesized
ttttgtggactgcttggctcaggg

SEQ ID NO: 37 - DNA, 22 bp, Artificial Sequence
  synthesized
caagattgtcagcaatgcatcc

SEQ ID NO: 38 - DNA, 22 bp, Artificial Sequence
  synthesized
atcacgccacagctttccagag

SEQ ID NO: 39 - DNA, 24 bp, Artificial Sequence
  synthesized
cgcagaatactggatgagcgaggt

SEQ ID NO: 40 - DNA, 24 bp, Artificial Sequence
  synthesized
gccggtgggtttcttcttcttgaa

SEQ ID NO: 41 - DNA, 25 bp, Artificial Sequence
  synthesized
ctcatggactgattatggacaggac

SEQ ID NO: 42 - DNA, 25 bp, Artificial Sequence
  synthesized
gcaggtcagcaaagaacttatagcc

SEQ ID NO: 43 - DNA, 23 bp, Artificial Sequence
  synthesized
gaaactggccacaatttactggt

SEQ ID NO: 44 - DNA, 20 bp, Artificial Sequence
  synthesized
ctgaaactctccgccacgta

SEQ ID NO: 45 - DNA, 20 bp, Artificial Sequence
  synthesized
gctcacccgctctttacctt

SEQ ID NO: 46 - DNA, 20 bp, Artificial Sequence
  synthesized
gatgtcgatggtgcgaaagc

SEQ ID NO: 47 - DNA, 20 bp, Artificial Sequence
  synthesized
aataagggcgtcgtggtcag

SEQ ID NO: 48 - DNA, 21 bp, Artificial Sequence
  synthesized
agcctcagactcattggaagc

SEQ ID NO: 49 - DNA, 20 bp, Artificial Sequence
  synthesized
ccagagaaggatgcggactc

SEQ ID NO: 50 - DNA, 20 bp, Artificial Sequence
  synthesized
gtgtcgtcgtagctctagcc

SEQ ID NO: 51 - DNA, 21 bp, Artificial Sequence
  synthesized
gtgttgggaaacgaggtccag

SEQ ID NO: 52 - DNA, 20 bp, Artificial Sequence
  synthesized
tggtctagagttggacgcag

SEQ ID NO: 53 - DNA, 21 bp, Artificial Sequence
  synthesized
gtcccattgctgacaggatgc

SEQ ID NO: 54 - DNA, 20 bp, Artificial Sequence
  synthesized
cgagtcggttgcgtagatcc

SEQ ID NO: 55 - DNA, 20 bp, Artificial Sequence
  synthesized
gccagatgagtggtggtcag

SEQ ID NO: 56 - DNA, 22 bp, Artificial Sequence
  synthesized
gcatctttccccattatgctcg

SEQ ID NO: 57 - DNA, 22 bp, Artificial Sequence
  synthesized
agctcatttcctggtatgacaa

SEQ ID NO: 58 - DNA, 21 bp, Artificial Sequence
  synthesized
ggtattcgagagaagggaggg